(19) United States
(12) Patent Application Publication
(10) Pub. No.: US 2004/0137516 A1
(43) Pub. Date: Jul. 15, 2004

(54) DNA sequences from staphylococcus aureus bacteriophage 44AHJD that encode anti-microbial polypeptides

(75) Inventors: Jerry Pelletier, Baie-D'Urfe (CA); Philippe Gros, St. Lambert (CA); Michael DuBow, Antony (FR); Dominique Bergeron, Montreal (CA)

Correspondence Address: SUGHRUE MION, PLLC, 2100 PENNSYLVANIA AVENUE, N.W., SUITE 800, WASHINGTON, DC, 20037, US

(73) Assignee: Phagetech, Inc.

(21) Appl. No.: 10/449,830

(22) Filed: May 31, 2003

Related U.S. Application Data
(63) Continuation-in-part of application No. 09/727,892, filed on Dec. 1, 2000.
(63) Continuation of application No. PCT/CA01/01754, filed on Nov. 30, 2001.
(60) Provisional application No. 60/168,777, filed on Dec. 1, 1999.

(51) Int. Cl. G01N 33/53; C12N 1/22

(52) U.S. Cl. 435/7.1

(57) ABSTRACT

This invention relates to newly identified polynucleotides and polypeptides, and their production and uses, as well as their variants, agonists and antagonists, and their uses. In particular, the invention relates to specific interaction between the S. aureus STAAU_R2 related protein or specific regions thereof, and growth-inhibitory proteins encoded by the S. aureus bacteriophage genome. The invention relates to the use of these interaction target sites as the basis of drug screening assays.

### FIELD OF THE INVENTION

The invention relates to bacterial genes and proteins that are implicated in the process of DNA replication and also to bacteriophage genes and their protein products that interact with bacterial proteins of DNA replication. More particularly, the invention relates to compositions and methods involving an essential Staphylococcus aureus gene and its encoded protein STAAU_R2. In addition, the invention relates to screening assays to identify compounds which modulate the level and/or activity of STAAU_R2 and to such compounds.

### BACKGROUND OF THE INVENTION

The Staphylococci make up a medically important genera of microbes known to cause several types of diseases in humans. S. aureus is a Gram positive organism which can be found on the skin of healthy human hosts and it is responsible for a large number of bacteremias.

S. aureus has been successfully treated with the penicillin derivative Methicillin in the past, but is now becoming increasingly resistant (MRSA—Methicillin Resistant S. aureus) to this antibiotic [Harbath et al., (1998) Arch. Intern. Med. 158: 182-189]. For example, S. aureus endocarditis mortality can range from 26-45%, and combined β-lactam/aminoglycoside therapy is proving increasingly ineffective in disease eradication [Røder et al., (1999) Arch. Intern. Med. 159: 462-469].

It is no longer uncommon to isolate S. aureus strains which are resistant to most of the standard antibiotics, and thus there is an unmet medical need and demand for new anti-microbial agents, vaccines, drug screening methods, and diagnostic tests for this organism. Antibiotics can be grouped into broad classes of activities against surprisingly few targets within the cell. Generally, the target molecule is a cellular protein that provides an essential function. The inhibition of activity of the essential protein leads either to death of the bacterial cell or to its inability to proliferate. Critical cellular functions against which antibiotics are currently in use include cell wall synthesis, folate and fatty acid metabolism, protein synthesis, and nucleic acid synthesis.

A proven approach in the discovery of a new drug, referred to as target-based drug discovery to distinguish it from cell-based drug discovery, is to obtain a target protein and to develop in vitro assays to interfere with the biological function of the protein. Nucleic acid metabolism is essential for all cells. The DNA synthesis machinery includes a number of proteins that act in concert to achieve rapid and highly processive replication of the chromosome in bacteria [reviewed in Kornberg, A., and Baker, T. A. 1992, DNA Replication, Second edition, New York: W.H. Freeman and Company, pp. 165-194]. As described below for DNA polymerase III, biological machines are often comprised of multiprotein complexes. Coordinated interactions among proteins of the bacterial primosome and replisome are essential to their efficiency. Thus, any members of essential multiprotein complexes are hypothetical targets for drug development. However, the fact that a protein can be associated with a certain biological function does not necessarily imply that it represents a suitable target for the development of new drugs [Drews J. 2000, Science 287:1960-1964]. For instance, although DNA replication is a well-known and essential process for bacterial growth, only a relatively small number of DNA replication proteins are targeted by currently-available antibiotics. Importantly, screening of compounds for those that inhibit the function of a target must be preferably rapid and selective.

There thus remains a need to identify new bacterial targets and new target domains, and more particularly S. aureus bacterial targets which could be used to screen for and identify antibacterial and more particularly anti-S. aureus agents. There also remains a need to identify new antimicrobial agents, vaccines, drug screening methods and diagnosis and therapeutic methods.

The present invention seeks to meet these and other needs.

The present description refers to a number of documents, the content of which is herein incorporated by reference in their entirety.

### SUMMARY OF THE INVENTION

The present invention relates to new antimicrobial agents, vaccines, drug screening methods and diagnosis and therapeutic methods.

More particularly, the invention relates to proteins which interact with STAAU_R2 and in particular to bacterial growth-inhibitory (or inhibitor) bacteriophage gene products that interacts with the S. aureus STMU₁₃ R2 polypeptide.

The invention also relates to a pair of interaction proteins and parts or fragments thereof. More specifically, the invention relates to the interacting domains of the S. aureus STAAU_R2 related protein and to proteins which interact with same and block or inhibit a STAAU_R2 biological activity. In a particular embodiment, the invention relates to a pair of interacting domains comprised of that of STAAU_R2 and a polypeptide encoded by a bacteriophage ORF which specifically interacts with STAAU_R2, such as the S. aureus bacteriophage 44AHJD ORF 25, Twort ORF168, or G1 ORF 240. In a particularly preferred embodiment of the present invention, the interaction of these domains and a modulation thereof forms the basis for screening assays to identify modulators of STAAU_R2 biological function and more particularly of antimicrobials.

The present invention also relates to polynucleotides and polypeptides of a multiprotein complex believed to be involved in DNA replication containing STAAU_R2 as a subunit, as well as variants and portions thereof.

In another aspect, the invention relates to methods for using such polypeptides and polynucleotides, including treatment and diagnosis of microbial diseases, amongst others.

In a further aspect, the invention relates to methods for identifying agonists and antagonists using the materials provided by the invention. In a related aspect, the invention relates to methods for treating microbial infections and conditions associated with such infections with the identified agonist or antagonist.

In a still further aspect, the invention relates to diagnostic assays for detecting diseases associated with microbial infections and conditions associated with such infections. In one embodiment, the diagnostic assay detects STAAU_R2 expression and/or activity.

In one particular embodiment of the invention, there is provided a method of identifying a compound that is active on a polypeptide comprising the amino acid sequence of SEQ ID NO: 2, biologically active fragment, or variant thereof, wherein SEQ ID NO:2, biologically active fragment, or variant thereof is capable of binding specifically with a polypeptide comprising the sequence selected from SEQ ID NOs:4, 6, 8 and 10, a biologically active fragment thereof, and variant thereof, wherein the fragment or variant retain their binding capability of specifically interacting with SEQ ID NO:2 or fragment or variant thereof.

In one preferred embodiment of the invention, the identification of a compound active on a STAAU_R2 polypeptide is provided by a method comprising: contacting a first and a second polypeptide in the presence or absence of a candidate compound, wherein the first polypeptide comprises the amino acid sequence of SEQ ID NO: 2, fragment or variant thereof which specifically bind to a second polypeptide derived from a bacteriophage ORF which is capable of binding specifically with SEQ ID NO:2, fragment, or variant thereof. In one particular embodiment, the second polypeptide is selected from the group consisting of a phage ORF (e.g. SEQ ID NO: 4 and 6), a fragment thereof (e.g. SEQ ID NO: 8) or variant thereof (e.g. SEQ ID NO 10), wherein this second polypeptide maintains its biological activity; and detecting a biological activity of the first and/or second polypeptide, wherein a decrease in the biological activity in the presence thereof relative to the biological activity in the absence of the candidate compound identifies the candidate compound as a compound that is active on a polypeptide comprising the amino acid sequence of SEQ ID NO:2, fragment or variant thereof.

In one particular embodiment, the biological activity is the binding of the first and second polypeptides to each other, the method comprising: contacting an assay mixture comprising a) a first polypeptide which comprises the amino acid sequence of SEQ ID NO:2 or a biologically active fragment thereof, or variant thereof, and b) a second polypeptide selected from the group consisting of SEQ ID NO:4, 6, 8, 10, a fragment thereof, and a variant thereof; with a test compound; measuring the binding of the first and the second polypeptides in the presence of the candidate compound relative to the binding in the absence thereof and; determining the ability of the candidate compound to interact with a STAAU_R2 polypeptide or variant thereof, wherein a decrease in the binding of the first and the second polypeptides in the presence of the candidate compound that interacts with STAAU_R2, relative to the binding in the absence of the candidate compound, identifies the candidate compound as a compound that is active on a STAAU_R2 polypeptide.

In another embodiment of the present invention, there is provided a process for producing a pharmaceutical composition comprising: a) carrying out a screening assay of the present invention aimed at identifying a compound that is active on a STAAU_R2 polypeptide comprising the amino acid sequence of SEQ ID NO:2, biologically active fragment, or variant thereof, wherein the STAAU_R2 polypeptide is capable of binding specifically with a second polypeptide derived from a bacteriophage ORF, and wherein the screening assay enables the identification of a candidate compound as a compound that is active on a STAAU_R2 polypeptide; and b) mixing the compound identified in a) to a suitable pharmaceutical carrier. In a further embodiment of this process of producing a pharmaceutical composition, the process further includes a scaling-up of the preparation for isolating of the identified compound active on the STAAU_R2 polypeptide. In yet another embodiment of this process of producing a pharmaceutical composition, the pharmaceutical composition prepared comprises a derivative or homolog of the compound identified in a).

In yet another embodiment of the present invention, there is provided one of a use of a) a STAAU_R2 polypeptide comprising the amino acid sequence of SEQ ID NO:2, a biologically active fragment thereof or variant thereof, wherein SEQ ID NO:2, biologically active fragment thereof or variant thereof (e.g. the STAAU_R2 polypeptide) is capable of binding specifically to a polypeptide derived from a bacteriophage ORF, b) a composition comprising a pair of specifically interacting domains comprised of a polypeptide of STAAU_R2, biologically active fragment thereof or variant thereof and a polypeptide encoded by a bacteriophage ORF which specifically interacts with STAAU_R2; or c) an assay mixture comprising a first polypeptide which comprises the amino acid sequence of SEQ ID NO:2, biologically active fragment thereof or variant thereof and a second polypeptide encoded by a bacteriophage ORF which specifically interact with each other; for the identification of a compound that is active on a polypeptide comprising the amino acid sequence of SEQ ID NO:2, biologically active fragment thereof or variant thereof. In a particularly preferred embodiment of the present invention, the bacteriophage polypeptide sequence is selected from the group consisting of SEQ NOs: 4, 6, 8, 10, a fragment thereof and a variant thereof, wherein the fragment thereof or variant thereof retain their specific binding capability to STAAU_R2 polypeptide.

In one embodiment, the step of detecting comprises the step of measuring the binding of the first and second proteins, wherein the first or the second protein is directly or indirectly detectably labeled.

In different embodiments, the step of detecting comprises, but is no limited to, measurement by the method selected from the group consisting of fluorescence resonance energy transfer, fluorescence polarization changes, measurement by surface plasmon resonance, a scintillation proximity assay, a biosensor assay, and phage display.

In one embodiment, a library of compounds is used. Non-limiting examples of candidate compounds include a small molecule, a peptidomimetic compound, a peptide, and a fragment or derivative of a bacteriophage inhibitor protein.

In one embodiment, the candidate compound is a peptide synthesized by expression systems and purified, or artificially synthesized.

The invention also encompasses a method of identifying an antimicrobial agent comprising determining whether a test compound is active on a S. aureus polypeptide, namely STAAU_R2 as set forth in SEQ ID NO: 2, or parts thereof.

In a further embodiment, identifying a compound active on a STAAU_R2 polypeptide is provided by a method which comprises: contacting a candidate compound with a polypeptide comprising the amino acid sequence of SEQ ID NO: 2; a fragment thereof or a variant thereof, the fragment or variant retaining its biological activity (e.g. it specifically binds one of the sequences selected from SEQ ID NOs: 4, 6, 8, 10, fragment thereof, or variant thereof (SEQ ID NO: 10)), and detecting binding of the candidate compound thereto, wherein detection of binding is indicative that the compound is active on the polypeptide.

In different embodiments, the step of detecting includes measuring the binding of a candidate compound to the polypeptide, wherein the compound is directly or indirectly detectably labeled, by a method comprising, but not limited to, fluorescence resonance energy transfer, fluorescence polarization changes, measurement by surface plasmon resonance, scintillation proximity assay, biosensor assay, and phage display.

In one embodiment, a library of compounds is used. Non-limiting examples of candidate compound include a small molecule, a peptidomimetic compound, a peptide, and a fragment or derivative of a bacteriophage inhibitor protein.

In one embodiment, the candidate compound is a peptide synthesized by expression systems and purified, or artificially synthesized.

The invention further encompasses a method of identifying a compound that is active on a STAAU_R2 polypeptide, comprising the steps of contacting a candidate compound (or library thereof) with cells expressing a polypeptide comprising SEQ ID NO: 2; and detecting STAAU_R2 activity in the cells, wherein a decrease in activity relative to STAAU_R2 activity in cells not contacted with a candidate compound is indicative of inhibition of STAAU_R2 activity. The invention also encompasses such a method but using a fragment or variant of SEQ ID NO:2.

Of course, the invention further encompasses methods of identifying a compound that modulates the activity of a STAAU_R2 polypeptide, wherein a compound increasing the activity relative to STAAU_R2 activity in cells not contacted with the candidate compound, is selected as a compound which is a stimulator of STAAU_R2 activity.

In a preferred embodiment, the step of detecting comprises a method of measuring the ability of a candidate, test compounds, or agents to stimulate or preferably to inhibit a STAAU_R2 molecule's ability to modulate DNA synthesis (such assays are described in more detail hereinbelow).

The invention further encompasses a method of identifying a compound that is active on a STAAU_R2 polypeptide, comprising the steps of contacting a candidate compound (or library thereof) in a cell-free assay, with a STAAU_R2 protein or biologically active portion thereof, either naturally occurring or recombinant in origin; and detecting STAAU_R2 activity, wherein a decrease in activity relative to STAAU_R2 activity in cell-free assay not contacted with a candidate compound is indicative of inhibition of STAAU_R2 activity.

In a preferred embodiment, the step of detecting comprises a method of measuring the ability of a candidate compound, test compounds, or agents to stimulate, or preferably to inhibit a STAAU_R2 molecule's ability to modulate DNA synthesis (such assays are described in more detail hereinbelow).

The invention further encompasses an agonist or an antagonist of the activity of a STAAU_R2 polypeptide or a nucleic acid or gene encoding the polypeptide.

The assays described herein may be used as initial or primary screens to detect promising lead compounds for further development. The same assays may also be used in a secondary screening assay to measure the activity of candidate compounds on a STAAU_R2 polypeptide. Often, lead compounds will be further assessed in additional, different screens. This invention also includes secondary STAAU_R2 screens which may involve biological assays utilizing S. aureus strains or other suitable bacteria.

Tertiary screens may involve the study of the effect of the agent in an animal. Accordingly, it is within the scope of this invention to further use an agent identified as described herein in an appropriate animal model. For example, an test compound identified as described herein (e.g., a STAAU_R2 inhibiting agent, an antisense STAAU_R2 nucleic acid molecule, a STAAU_R2-specific antibody, or a STAAU_R2-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatment (e.g. bacterial infections), as described herein.

The invention further encompasses a method of making an antibacterial compound, comprising the steps of: a) determining whether a candidate compound is active on a polypeptide comprising the amino acid sequence of SEQ ID NO: 2, fragment or variant thereof, or a gene encoding the polypeptide; and b) synthesizing or purifying the candidate compound in an amount sufficient to provide a therapeutic effect when administered to an organism infected by a bacterium naturally producing a polypeptide comprising the amino acid sequence of SEQ ID NO: 2, fragment or variant thereof.

The invention further encompasses a method for inhibiting a bacterium, comprising contacting the bacterium with a compound active on a polypeptide comprising the amino acid sequence of SEQ ID NO: 2, fragment or variant thereof, or a nucleic acid encoding the polypeptide.

In one embodiment, the step of contacting is performed in vitro.

In another embodiment, the step of contacting is performed in vivo in an animal.

In another embodiment, bacterium is contacted with the active compound in combination with existing antimicrobial agents. Thus, the invention also relates to antimicrobial compositions comprising a compound of the present invention in combination with an existing antimicrobial agent. Of course, more than one active compound of the present invention could be combined with or without existing antimicrobial agent(s).

The invention further encompasses a method for treating or preventing a bacterial infection in an animal suffering from an infection or susceptible of suffering from same, comprising administering thereto a therapeutically effective amount of a compound active on a polypeptide comprising the amino acid sequence of SEQ ID NO: 2, variant or fragment thereof, or nucleic acid sequence encoding same. The animal is preferably, but not necessarily a mammal, and more preferably a human. In one embodiment, the active compound is administred to the animal in combination with existing antimicrobial agents. Thus, the invention also relates to antimicrobial compositions comprising a compound of the present invention in combination with an existing antimicrobial agent.

The invention further encompasses a method of prophylactic treatment to prevent bacterial infection comprising contacting an indwelling device with a compound active on a polypeptide comprising the amino acid sequence of SEQ ID NO: 2, variant or fragment thereof before its implantation into a mammal, such contacting being sufficient to prevent S. aureus infection at the site of implantation.

The invention further encompasses a method of prophylactic treatment to prevent infection of an animal by a bacterium comprising administering to the animal a prophylactically effective amount of a compound that is active on a polypeptide comprising the amino acid sequence of SEQ ID NO: 2, variant or fragment thereof or a gene encoding the polypeptide in an amount sufficient to prevent infection of the animal. In a particular embodiment, the prophylactically effective amount reduces adhesion of the bacterium to a tissue surface of the mammal.

The invention further encompasses a method of diagnosing in an animal an infection with S. aureus, comprising: determining the presence in the animal of a polypeptide comprising the amino acid sequence of SEQ ID NO: 2, part thereof, variant thereof, fragment thereof, epitope thereof or nucleic acid encoding same. Preferably the polypeptide is capable of specifically interacting with at least one of 44AHJD ORF 25, Twort ORF168 or G1 ORF 240. Preferably, the animal is a human.

In one embodiment, the determining step comprises contacting a biological sample of the animal or individual with an antibody specific for an epitope present on a polypeptide comprising the amino acid sequence of SEQ ID NO: 2, variant or fragment thereof.

The invention further encompasses a method of diagnosing in an animal or individual an infection with Staphylococcus aureus, comprising determining the presence in the animal or individual of a nucleic acid sequence encoding a polypeptide comprising the amino acid sequence of SEQ ID NO: 2, variant or fragment thereof, wherein the polypeptide is capable of specifically interaction with at least one of 44AHJD ORF 25, Twort ORF168 or G1 ORF 240.

In one embodiment, the determining step comprises contacting a nucleic acid sample of the animal or individual with an isolated, purified or enriched nucleic acid probe of at least 15 nucleotides in length that hybridizes under stringent hybridization conditions with the sequence of SEQ ID NO: 1, or the complement thereof.

The invention further encompasses a composition comprising two polypeptides, a bacteriophage-encoded polypeptide and a S. aureus STAAU_R2 polypeptide corresponding to SEQ ID NO: 2. In another embodiment, the invention encompasses a composition comprising two interacting polypeptides derived from a bacteriophage encoded polypeptide and a S. aureus STAAU_R2 polypeptide. As such, the invention encompasses a composition comprising two nucleic acid sequences encoding these interacting polypeptides.

Further features and advantages of the invention will become more fully apparent in the following description of the embodiments and drawings thereof, and from the claims.

### BRIEF DESCRIPTION OF THE DRAWINGS

Having thus generally described the invention, reference will now be made to the accompanying drawings, showing by way of illustration a preferred embodiment thereof, and in which:

FIG. 1 shows the nucleotide (SEQ ID NO: 1) and amino acid (SEQ ID NO: 2) sequences of S. aureus STAAU_R2.

FIG. 2 shows the nucleotide and the amino acid sequences of S. aureus bacteriophage 44AHJD ORF 25, Twort ORF168, a 36 amino acids fragment derived from Twort ORF 168 and G1 ORF 240.

FIG. 3 shows the bacterial inhibitory potential of bacteriophage 44AHJD ORF 25 or Twort ORF168 and the expression vector used to induce their expression in S. aureus. A) Schematic diagram of arsenite-inducible expression vector (e.g. for pT/ORF and pTM/ORF) used to induce expression of 44AHJD ORF 25 or Twort ORF168 in S. aureus cells; B) Results of an assay for inhibitory potential of 44AHJD ORF 25 or Twort ORF168 when expressed in S. aureus grown in liquid medium followed by plating on semi-solid medium either containing or not containing the antibiotic (30 ug/ml of kanamycin) necessary to maintain the selective pressure for the plasmid.

FIG. 4 depicts the results from affinity chromatography using GST and GST/44AHJD ORF 25 as ligands with a S. aureus extract prepared by French pressure cell lysis and sonication. Eluates from affinity columns containing the GST and GST/ORF25 ligands at 0, 0.1, 0.5, 1.0, and 2.0 mg/ml resin were resolved by SDS-12.5% PAGE. Proteins were visualized by silver staining. Micro-columns were sequentially eluted with 100 mM ACB containing 0.1% Triton X-100, 1 M NaCl ACB, and 1% SDS (SDS-PAGE only shows for 1% SDS). Each molecular weight marker is approximately 100 ng. The lanes labeled ACB indicate eluates from a 2.0 mg/ml ligand column loaded only with ACB buffer containing 75 mM NaCl. The arrow designated PT 48 indicates protein specifically interacting with 44AHJD ORF 25. Band corresponding to PT 48 was excised for protein identification.

FIG. 5 shows results of a tryptic peptide mass spectrum of the PT 48 protein that interacted with 44AHJD ORF 25 and that was eluted with 1% SDS. The control band (designated PT48C in FIG. 4) excised from the 48 kDA region did not contain PT 48.

FIG. 6 shows the identification of PT 48 (herein STAAU_R2) as S. aureus DNA-directed DNA polymerase III beta subunit protein from the Genbank database (accession number: GI:15922992).

FIG. 7 shows affinity chromatography using GST/Twort ORF 168 (A) or GST (B) as ligands with a 5.0 mg/ml S. aureus extract. Eluates from affinity columns containing the ligands at 0, 0.1, 0.5, 1.0, and 2.0 mg/ml resin were resolved by 14% SDS-PAGE and the gel was stained with silver nitrate. Micro-columns were sequentially eluted with 100 mM ACB containing 0.1% Triton X-100 (SDS-PAGE not shown), 1 M NaCl ACB, and 1% SDS. Each molecular weight marker is approximately 200 ng. The lanes labeled ACB indicate eluates from a 2.0 mg/ml ligand column loaded only with ACB buffer containing 100 mM NaCl. The arrow designated PT50 indicates protein specifically interacting with Twort ORF 168. Band corresponding to PT50 was excised for protein identification.

FIG. 8 shows the tryptic peptide mass spectrum analysis of the PT50 protein interacting with Twort ORF 168. The gel slice containing PT50 contained one protein.

FIG. 9 shows the identification of PT50 (herein STAAU_R2) as S. aureus DNA-directed DNA polymerase III beta subunit protein from the Genbank database (accession number: GI:15922992).

FIG. 10 shows schematic representations of A) the procedure for cloning S. aureus STAAU_R2 in the yeast expression vector pGADT7 (pGADSTAAU_R2); B) the procedure for cloning phage Twort ORF 168 in the yeast expression vector pGBKT7 (pGBK TwortORF168); and C) the yeast two-hybrid system in three stylized cells expressing either GADSTAAU_R2 (top panel), Twort ORF 168 (middle panel), or both GADSTAAU_R2 and Twort ORF 168 (bottom panel).

FIG. 11 shows the results of yeast two hybrid analyses designed to test the interaction of S. aureus STAAU_R2 comprising the amino acid of SEQ ID NO: 2 and Twort ORF 168. A) and B). Yeasts were co-transformed with pairs of vectors as indicated above each pair of photographs of Petri plates. Co-transformants were plated in parallel on yeast synthetic medium (SD) supplemented with amino acid drop-out lacking tryptophan and leucine (TL minus) and on SD supplemented with amino acid drop-out lacking tryptophan, histidine, adenine and leucine (THAL minus). Co-transformants harboring the Twort ORF 168 polypeptide only grew on selective THAL minus media in the presence of STAAU_R2 (top pairs of petri plates). Co-transformation of these polypeptides with control vectors harboring non-interacting proteins (pGBKLaminC or pGADT7-T) does not result in growth on THAL minus medium. C) Results of the luminescent β-galactosidase enzymatic assays with protein extracts from the indicated co-transformants.

FIG. 12 shows optimal global and local alignments of Twort ORF168 (SEQ ID NO:6) and the STAAU_R2 interaction domain of Twort ORF168 (SEQ ID NO:8) with that of G1 ORF 240 (SEQ ID NO: 10).

Other objects, advantages and features of the present invention will become more apparent upon reading of the following non-restrictive description of preferred embodiments with reference to the accompanying drawing which is exemplary and should not be interpreted as limiting the scope of the present invention.

### DESCRIPTION OF THE PREFERRED EMBODIMENT

The invention relates to the discovery of an essential gene and its encoded polypeptide in S. aureus and portions thereof useful for example in screening, diagnostics, and therapeutics. More specifically, the invention also relates to S. aureus STAAU_R2 polypeptides and polynucleotides as described in greater detail below, and to a pair of polynucleotides encoding a pair of interacting polypeptides, to the pair of polypeptides themselves, or interacting domains thereof. In a particular embodiment, the pair includes a S. aureus STAAU_R2 polypeptide or interacting domain thereof and a) a 44AHDJ ORF 25 or interacting domain thereof; b) a Twort ORF168 or interacting domain thereof; or c) a G1 ORF 240 or interacting domain thereof. In one embodiment, the invention relates to STAAU_R2 having the nucleotide and amino acid sequences disclosed as SEQ ID NO: 1 and SEQ ID NO: 2, respectively. The sequences presented as SEQ ID NOs: 1 and 2 represent an exemplification of the invention, since those of ordinary skill will recognize that such sequences can be usefully employed in polynucleotides in general, including ribopolynucleotides.

The methodology of two previous inventions (U.S. Provisional Patent Application 60/110,992, filed Dec. 3, 1998, and PCT International Application WO1999/IB99/02040, filed Dec. 3, 1999) has been used to identify and characterize essential polynucleotide and polypeptide sequences from S. aureus.

Thus, in a particular embodiment of the present invention, the present invention provides polynucleotide and polypeptide sequences isolated from S. aureus that can be used in a drug screening assay to identify compounds with anti-microbial activity. The polynucleotide and polypeptide sequences can be isolated using a method similar to those described herein, or using another method. In addition, such polynucleotide and polypeptide-sequences can be chemically synthesized. The identification of these S. aureus sequences as targets for three different bacteriophages validates the approach of the present invention to identify bacterial targets and also validates STAAU_R2 as a key target for antibacterial drug development as well as diagnosis and treatment methods based thereon.

Definitions

In order to provide a clear and consistent understanding of terms used in the present description, a number of definitions are provided hereinbelow.

The terminology “active on”, with reference to a particular cellular target, such as the product of a particular gene, means that the target is an important part of a cellular pathway which includes that target and that an agent or compound acts on that pathway. Thus, in some cases the agent or compound may act on a component upstream or downstream of the stated target (i.e. indirectly on the target), including a regulator of that pathway or a component of that pathway. In general, an antibacterial agent is active on an essential cellular function, often on a product of an essential gene (i.e. directly on the target).

The terminology “active on” also refers to a measurable effect of the compound on the target it is active on (as compared to the activity of the target in the absence of the compound). The activity referred thereto is any one of a biological activity of one of the polypeptides of the present invention.

As used herein, the terms “inhibit”, “inhibition”, “inhibitory”, and “inhibitor” all refer to a function of reducing a biological activity or function. Such reduction in activity or function can, for example, be in connection with a cellular component (e.g., an enzyme), or in connection with a cellular process (e.g., synthesis of a particular protein), or in connection with an overall process of a cell (e.g., cell growth). In reference to cell growth, the inhibitory effects may be bacteriocidal (killing of bacterial cells) or bacteriostatic (i.e.—stopping or at least slowing bacterial cell growth). The latter slows or prevents cell growth such that fewer cells of the strain are produced relative to uninhibited cells over a given time period. From a molecular standpoint, such inhibition may equate with a reduction in the level of, or elimination of, the transcription and/or translation and/or stability of a specific bacterial target(s), and/or reduction or elimination of activity of a particular target biomolecule.

As used herein, the term “STAAU_R2 polypeptide” “dnaN polypeptide” refers to a polypeptide encompassing S. aureus STAAU_R2 (SEQ ID NO: 2), variant thereof or an active domain of S. aureus STAAU_R2. As used herein, the term “active domain of S. aureus STAAU_R2”, “biologically active polypeptide of STAAU_R2” or the like refers to a polypeptide fragment or portion of S. aureus STAAU_R2 that retains an activity of S. aureus STAAU_R2. The term “STAAU_R2 polypeptide” is meant to encompass S. aureus STAAU_R2 or an active domain of S. aureus STAAU_R2 that is fused to another, non-STAAU_R2 polypeptide sequence.

“STAAU_R2 activity” “polypeptide comprising the amino acid sequence SEQ ID NO: 2 activity” “dnaN polypeptide activity” or “biological activity” of STAAU_R2 or other polypeptides of the present invention is defined as a detectable biological activity of a gene, nucleic acid sequence, protein or polypeptide of the present invention. This includes any physiological function attributable to the specific biological activity of STAAU_R2, or phage ORF of the present invention. This includes measurement of the DNA synthesis activities of STAAU_R2 in cells or in vitro. Non-limiting examples of the biological activities may be made directly or indirectly. STAAU_R2 biological activity, for example, is not limited, however, to its function in DNA synthesis. Biological activities may also include simple binding to other factors (polypeptides or otherwise), including compounds, substrates, and of course interacting proteins. Thus, for STAAU_R2, biological activity includes any standard biochemical measurement of STAAU_R2 such as conformational changes, phosphorylation status or any other feature of the protein that can be measured with techniques known in the art. STAAU_R2 biological activity also includes activities related to STAAU_R2 gene transcription or translation, or any biological activities of such transcripts or translation products. The instant invention is also concerned with STAAU_R2 interaction with an inhibitory polypeptide of the present invention, biological activity of STAAU_R2 also includes assays which monitor binding and other biochemical measurements of these polypeptides. Furthermore, for certainty, the terminology “biological activity” also includes measurements based on the interaction of domains of interacting proteins of the present invention (i.e. the phage ORFs or domains thereof). Non-limiting examples of “biological activity” include one or more of the following:

i) Binding to bacterial growth inhibitory ORFs derived from bacteriophage including 44AHDJ ORF 25, Twort ORF168, G1 ORF 240 polypeptides or part thereof.

Determining the binding between polypeptides of the present invention can be accomplished by one of the methods described below or known in the art for determining direct binding. While it might be advantageous in certain embodiments of the present invention to provide a binding assay which is amenable to automation and more particularly to high-throughput, the present invention is not so limited. The binding or physical interaction of a polypeptide comprising the amino acid sequence of SEQ ID NO: 2, provided herein, or fragment thereof to bacteriophage protein 44AHDJ ORF 25, Twort ORF168, or G1 ORF 240 or portion thereof (e.g. SEQ ID NO: 8). The interaction or binding of a polypeptide comprising the amino acid sequence of SEQ ID NO: 2 and a binding portion of bacteriophage 44AHDJ ORF 25, Twort ORF168, G1 ORF 240 may be between isolated polypeptides consisting essentially of the sequence necessary for binding, or, alternatively, the respective polypeptide sequence may be comprised within a larger polypeptide.

A number of non-limiting methods, useful in the invention, to measure the binding of bacteriophage 44AHDJ ORF 25, Twort ORF168, G1 ORF 240 to a polypeptide comprising the amino acid sequence of SEQ ID NO: 2, or fragment thereof are described below. Binding can be measured by coupling one molecule to a surface or support such as a membrane, a microtiter plate well, or a microarray chip, and monitoring binding of a second molecule by any number of means including optical spectroscopy, fluorometry, and radioactive label detection.

For example, Fluorescence Resonance Energy Transfer (FRET), in which the close proximity of two fluorophores, whether intrinsic to, as in the case of a naturally-fluorescent amino acid residue such as tryptophan, or either covalently or non-covalently bound to a separate molecule, causes the emission spectrum of one fluorophore to overlap with the excitation spectrum of the second, and thus dual fluorescence following excitation of only one fluorophore is indicative of binding. For example, Fluorescence Resonance Energy Transfer (FRET), in which the close proximity of two fluorophores, whether intrinsic to, as in the case of a naturally-fluorescent amino acid residue such as tryptophan, or either covalently or non-covalently bound to a separate molecule, causes the emission spectrum of one fluorophore to overlap with the excitation spectrum of the second, and thus dual fluorescence following excitation of only one fluorophore is indicative of binding. An additional assay useful in the present invention is fluorescence polarization, in which the quantifiable polarization value for a given fluorescently-tagged molecule is altered upon binding to a second molecule. Surface plasmon resonance assays can be used as a quantitative method to measure binding between two molecules by the change in mass near an immobilized sensor caused by the binding of one protein from the aqueous phase to a second immobilized on the sensor. A scintillation proximity assay can also be used to measure binding of a polypeptide comprising the amino acid sequence of SEQ ID NO: 2, and fragment thereof and a bacteriophage ORF or fragment thereof in which binding in the proximity to a scintillant converts radioactive particles into a photon signal that is detected by a scintillation counter or other detector. Additionally, binding can be evaluated by a Bio Sensor assay, which is based on the ability of the sensor to register changes in admittance induced by ion-channel modulation following binding. Phage display is also a powerful quantitative assay to measure protein:protein interaction using colourimetric ELISA (enzyme-linked immunosorbent assay).

ii) Stimulation of DNA Synthesis

The biological activity also relates to DNA synthesis stimulation of a polypeptide having the S. aureus STAAU_R2 sequence provided herein, a fragment or variant thereof, or a protein comprising a S. aureus STAAU_R2 polypeptide that directly interacts with bacteriophage protein 44AHJD ORF 25, Twort ORF168, G1 ORF 240, or a STAAU_R2-binding fragment of the 44AHJD ORF 25, Twort ORF168, G1 ORF 240 proteins or variant thereof.

A number of methods, useful in the invention, to measure the DNA synthesis stimulation of a polypeptide comprising the amino acid sequence of STAAU_R2 are described below. The level of DNA synthesis can be evaluated for example by the measurement of radiolabeled nucleotides incorporation into DNA of S. aureus cells.

The rate and processivity of DNA synthesis could also be measured by using soluble in vitro systems based on the use of a variety of different synthetic DNA substrates including single-stranded (ss) DNA, double-stranded (ds) DNA, either linear or circular. In one embodiment, the replication assay involves crude or partially purified cellular proteins extracts or recombinantly produced proteins. In another embodiment, the reconstituted protein assays involves partially purified or pure forms of native proteins or fusion proteins or fragments thereof.

iii) Loading onto DNA

The DNA loading of a polypeptide having the S. aureus STAAU_R2 sequence provided herein, a fragment or analog thereof or a protein comprising a S. aureus STAAU_R2 polypeptide that directly interacts with one of bacteriophage 44AHJD ORF 25, Twort ORF168, G1 ORF 240 proteins, or a STAAU_R2-binding fragment of the 44AHJD ORF 25, Twort ORF168, G1 ORF 240 proteins or variant thereof can also be monitored. In one embodiment, an in vitro reconstituted assay involves the measurement of ³²P-labeled or fluorescently-labeled STAAU_R2 assembly onto a circular DNA substrate.

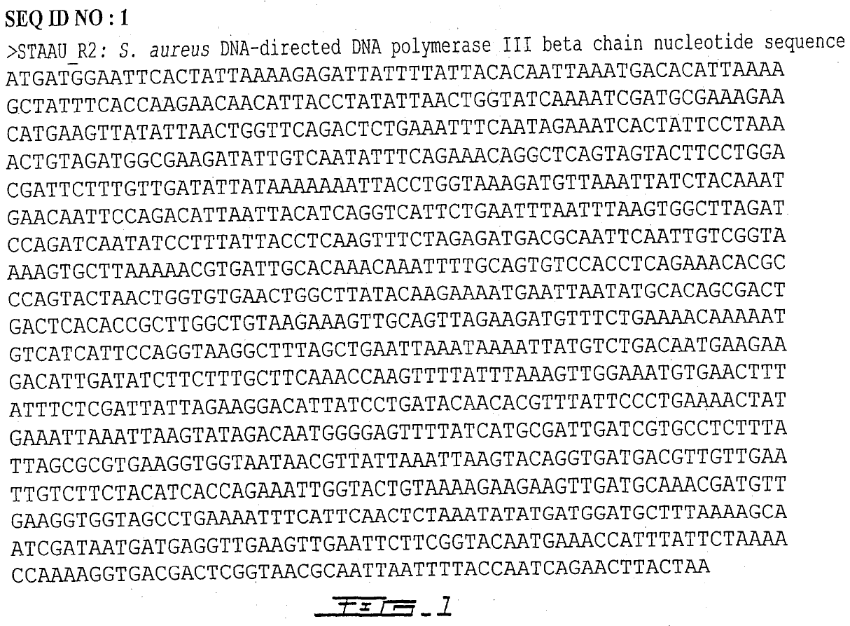

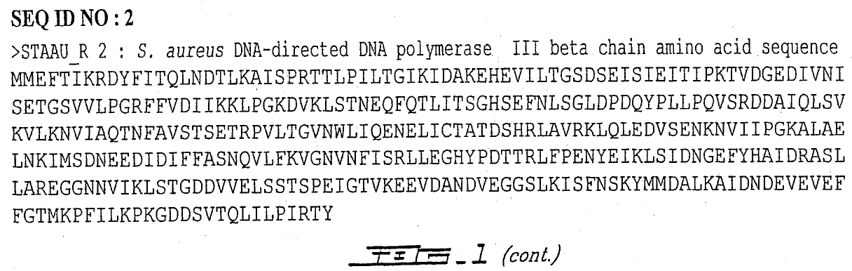

As used herein, the term “polynucleotide encoding a polypeptide” or equivalent language encompasses polynucleotides that include a sequence encoding a polypeptide of the invention, particularly a bacterial polypeptide and more particularly a polypeptide of S. aureus STAAU_R2 protein having an amino acid sequence set out in FIG. 1, SEQ ID NO: 2. The term also encompasses polynucleotides that include a single continuous region or discontinuous regions encoding the polypeptide (for example, polynucleotides interrupted by integrated phage, an integrated insertion sequence, an integrated vector sequence, an integrated transposon sequence, or otherwise altered due to RNA editing or genomic DNA reorganization) together with additional regions that also may contain coding and/or non-coding sequences.

As used herein, the term “STAAU_R2 gene” “DnaN gene” is meant to encompass a polynucleotide encoding a S. aureus STAAU_R2 polypeptide. Any additional nucleotide sequences necessary to direct transcription of RNA encoding a S. aureus STAAU_R2 polypeptide, either in a cell or in vitro, will be termed “regulatory sequences”, which include but are not limited to transcriptional promoters and enhancers, and transcription terminators.

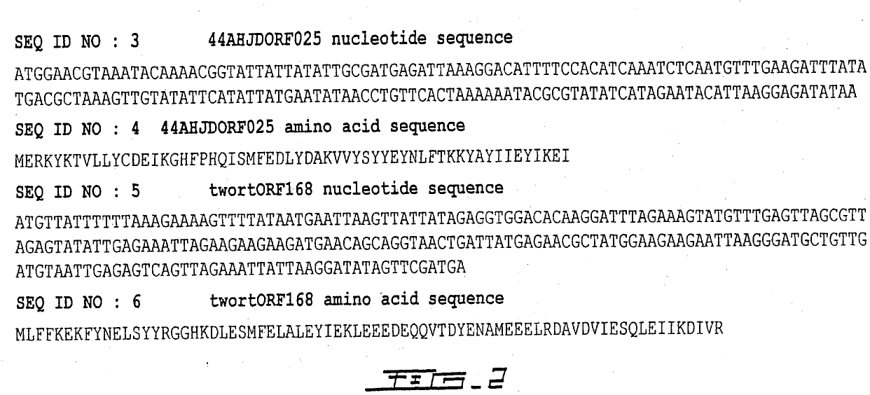

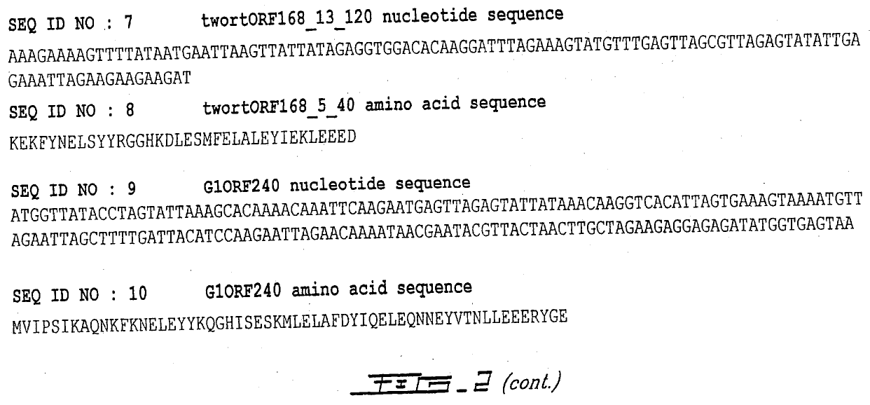

As used herein, the term “ORF 25” or “phage 44AHJD ORF 25” or “44AHJD ORF 25” encompasses a polynucleotide having the sequence provided in FIG. 2 (SEQ ID NO: 3), which encodes a gene product known as the 44AHJD ORF 25 gene product.

As used herein, the term “ORF 168” or “phage Twort ORF 168” or “Twort ORF 168” encompasses a polynucleotide having the sequence provided in FIG. 2 (SEQ ID NO: 5), which encodes a gene product known as the Twort ORF 168 gene product.

As used herein, the term “ORF 240” or “phage G1 ORF 240” encompasses a polynucleotide having the sequence provided in FIG. 2 (SEQ ID NO: 9). which encodes a gene product known as the G1 ORF 240 gene product.

As used herein, the term “polynucleotide(s)” generally refers to any polyribonucleotide or poly-deoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s). Polynucleotides can also be DNA and RNA chimeras.

As used herein, the term “polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene-encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may contain many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of cysteine, formation of pyroglutamate, formylation, gamma-carboxylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation, selenoylation, sulfation and transfer-RNA mediated addition of amino acids to proteins, such as arginylation, and ubiquitination. See, for instance: Proteins—Structure and Molecular Properties, 2nd Ed., T. E. Creighton, W. H. Freeman and Company, New York (1993); Wold, F., Posttranslational Protein Modifications: Perspectives and Prospects, pgs. 1-12 in Posttranslational Covalent Modification of Proteins, B. C. Johnson, Ed., Academic Press, New York (1983); Seifter et al., Meth. Enzymol. 182:626-646 (1990); and Rattan et al., Protein Synthesis: Posttranslational Modifications and Aging, Ann. N.Y. Acad. Sci. 663: 48-62(1992). Polypeptides may be branched or cyclic, with or without branching. Cyclic, branched and branched circular polypeptides may result from post-translational natural processes and may be made by entirely synthetic methods, as well.

As used herein, the term “variant(s)” refers to a polynucleotide or polypeptide that differs from a reference polynucleotide or polypeptide, respectively, but retains one or more of the biological activities of the initial (e.g. non-variant) polynucleotide or polypeptide of the present invention (e.g. STAAU_R2). A typical variant of a polynucleotide differs in nucleotide sequence from another reference polynucleotide. Changes in the nucleotide sequence of the variant may or may not alter the amino acid sequence of a polypeptide encoded by the reference polynucleotide. Nucleotide changes may result in amino acid substitutions, additions, deletions, and truncations in the polypeptide encoded by the reference sequence, or in the formation of fusion proteins, as discussed below. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. The present invention also includes variants of each of the polypeptides of the invention, that is polypeptides that vary from the referents by conservative amino acid substitutions whereby a residue is substituted by another with like characteristics. Typically, such substitutions are among Val, Leu and Ile; among Ser and Thr; among the acidic residues Asp and Glu; and among the basic residues Lys and Arg; or aromatic residues Phe and Tyr. Particularly preferred are variants in which 1-10, 1-5,1-3, 2-3, or 1 amino acid or amino acids are substituted, deleted, or added in any combination. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

As used herein, the term “fragment”, when used in reference to a polypeptide, is a polypeptide having an amino acid sequence that is entirely the same as part but not all of the amino acid sequence of the polypeptide according to the invention from which it “derives”. As with S. aureus STAAU_R2 polypeptides, fragments may be “free-standing” (“consisting of”), or comprised within a larger polypeptide of which they form a part or region, most preferably as a single continuous region in a single larger polypeptide.

The term “isolated”, when used in reference to a nucleic acid means that a naturally occurring sequence has been removed from its normal cellular (e.g., chromosomal) environment or is synthesized in a non-natural environment (e.g., artificially synthesized). Thus, the sequence may be in a cell-free solution or placed in a different cellular environment. The term does not imply that the sequence is the only nucleotide chain present, but that it is essentially free (about 90-95% pure at least) of non-nucleotide material naturally associated with it, and thus is distinguished from isolated chromosomes.

The term “enriched”, when used in reference to a polynucleotide means that the specific DNA or RNA sequence constitutes a significantly higher fraction (2-5 fold) of the total DNA or RNA present in the cells or solution of interest than in normal or diseased cells or in cells from which the sequence was originally taken. This could be caused by a person, by preferential reduction in the amount of other DNA or RNA present, or by a preferential increase in the amount of the specific DNA or RNA sequence, or by a combination of the two. However, it should be noted that enriched does not imply that there are no other DNA or RNA sequences present, just that the relative amount of the sequence of interest has been significantly increased.

As used herein, the term “significantly higher fraction” indicates that the level of enrichment is useful to the person making such an enrichment and indicates an increase in enrichment relative to other nucleic acids of at least about 2-fold, or 5- to 10-fold or even more. The term also does not imply that there is no DNA or RNA from other sources. The other source of DNA may, for example, comprise DNA from a yeast or bacterial genome, or a cloning vector such as pUC19. This term distinguishes from naturally occurring events, such as viral infection, or tumor type growths, in which the level of one mRNA may be naturally increased relative to other species of mRNA. That is, the term is meant to cover only those situations in which a person has intervened to elevate the proportion of the desired nucleic acid.

As used herein, the term “purified” in reference to nucleic acid does not require absolute purity (such as a homogeneous preparation). Instead, it represents an indication that the sequence is relatively more pure than in the natural environment (compared to the natural level, this level should be at least 2-5 fold greater, e.g., in terms of mg/mL). Individual clones isolated from a genomic or cDNA library may be purified to electrophoretic homogeneity. The claimed DNA molecules obtained from these clones could be obtained directly from total DNA or from total RNA. cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁶-fold purification of the native message over its proportion in naturally occurring cells. Thus, purification of at least one order of magnitude, preferably two or three orders, and more preferably four or five orders of magnitude is expressly contemplated. A genomic library can be used in the same way and yields the same approximate levels of purification.

The terms “isolated”, “enriched”, and “purified” used with respect to nucleic acids, above, may similarly be used to denote the relative purity and abundance of polypeptides. These, too, may be stored in, grown in, screened in, and selected from libraries using biochemical techniques familiar in the art. Such polypeptides may be natural, synthetic or chimeric and may be extracted using any of a variety of methods, such as antibody immunoprecipitation, other “tagging” techniques, conventional chromatography and/or electrophoretic methods. Some of the above utilize the corresponding nucleic acid sequence.

As used herein, the term “complement” when used in reference to a given polynucleotide sequence refers to a sequence of nucleotides which can form a double-stranded heteroduplex in which every nucleotide in the sequence of nucleotides is base-paired by hydrogen bonding to a nucleotide opposite it in the heteroduplex with the given polynucleotide sequence. The term may refer to a DNA or an RNA sequence that is the complement of another RNA or DNA sequence. As used herein, the term “hybridizes” refers to the formation of a hydrogen-bonded heteroduplex between two nucleic acid molecules. Generally, a given nucleic acid molecule will hybridize with its complement, or with a molecule that is sufficiently complementary to the given molecule to permit formation of a hydrogen-bonded heteroduplex between the two molecules.

As used herein, the term “probe” refers to a polynucleotide of at least 15 nucleotides (nt), 20 nt, 30 nt, 40 nt, 50 nt, 75 nt, 100 nt, 200 nt, 500 nt, 1000 nt, and even up to 5000 to 10,000 nt in length.

“Identity” and “similarity,” as used herein and as known in the art, are relationships between two or more polypeptide sequences or two or more polynucleotide sequences, as the case may be, as determined by comparing the sequences.

Amino acid or nucleotide sequence “identity” and “similarity” are determined from an optimal global alignment between the two sequences being compared. A non-limiting example of optimal global alignment can be carried-out using the Needleman-Wunsch algorithm (Needleman and Wunsch, 1970, J. Mol. Biol. 48:443453). “Identity” means that an amino acid or nucleotide at a particular position in a first polypeptide or polynucleotide is identical to a corresponding amino acid or nucleotide in a second polypeptide or polynucleotide that is in an optimal global alignment with the first polypeptide or polynucleotide. In contrast to identity, “similarity” encompasses amino acids that are conservative substitutions.

The term “conservative” substitution is well-known in the art and broadly refers to a substitution which does not significantly change the chemico-physical properties of the substituted amino acid. For example, a “conservative” substitution is any substitution that has a positive score in the blosum62 substitution matrix (Hentikoff and Hentikoff, 1992, Proc. Natl. Acad. Sci. USA 89:10915-10919). By the statement “sequence A is n % similar to sequence B” is meant that n % of the positions of an optimal global alignment between sequences A and B consists of conservative substitutions. By the statement “sequence A is n % identical to sequence B” is meant that n % of the positions of an optimal global alignment between sequences A and B consists of identical residues or nucleotides. Optimal global alignments in this disclosure used the following parameters in the Needleman-Wunsch alignment algorithm:

For polypeptides:

Substitution matrix: blosum62.

Gap scoring function: −A−B*LG, where A=11 (the gap penalty), B=1 (the gap length penalty) and LG is the length of the gap.

For nucleotide sequences:

Substitution matrix: 10 for matches, 0 for mismatches.

Gap scoring function: −A−B*LG where A=50 (the gap penalty), B=3 (the gap length penalty) and LG is the length of the gap.

The term ‘identity’ and ‘similarity’ between sequences can be extended to their fragments. An optimal local alignment between sequences A and B is the highest scoring alignment of fragments of A and B. By the statement “sequence A is n % identical locally to B” is meant that n % of the positions of an optimal local alignment between sequences A and B consists of conservative substitutions. By the statement “sequence A is n % similar locally to B” is meant that n % of the position of an optimal local alignment between sequences A and B consists of identical residues or nucleotides. An non-limiting example of optimal local alignment can be carried-out using the Smith-Waterman algorithm (Smith, T. F and Waterman, M. S. 1981. Identification of common molecular subsequences. J. of M. Biol. 147:195-197).

Of course, the above-listed parameters are but one specific example of alignment algorithm parameters. Numerous algorithms and parameters are available and known to the person of ordinary skill.

Typical conservative substitutions are among Met, Val, Leu and lie; among Ser and Thr; among the residues Asp, Glu and Asn; among the residues Gln, Lys and Arg; or aromatic residues Phe and Tyr. In calculating the degree (most often as a percentage) of similarity between two polypeptide sequences, one considers the number of positions at which identity or similarity is observed between corresponding amino acid residues in the two polypeptide sequences in relation to the entire lengths of the two molecules being compared.

As used herein, the term “antibody” is meant to encompass constructions using the binding (variable) region of such an antibody, and other antibody modifications. Thus, an antibody useful in the invention may comprise a whole antibody, an antibody fragment, a polyfunctional antibody aggregate, or in general a substance comprising one or more specific binding sites from an antibody. The antibody fragment may be a fragment such as an Fv, Fab or F(ab′)₂ fragment or a derivative thereof, such as a single chain Fv fragment. The antibody or antibody fragment may be non-recombinant, recombinant or humanized. The antibody may be of an immunoglobulin isotype, e.g., IgG, IgM, and so forth. In addition, an aggregate, polymer, derivative and conjugate of an immunoglobulin or a fragment thereof can be used where appropriate. Neutralizing antibodies are especially useful according to the invention for diagnostics, therapeutics and methods of drug screening and drug design.

As used herein, the term “specific for an epitope present on a S. aureus STAAU_R2 polypeptide”, when used in reference to an antibody, means that the antibody recognizes and binds an antigenic determinant present on a S. aureus STAAU_R2 polypeptide according to the invention.

As used herein, the term “antigenically equivalent derivative(s)” encompasses a polypeptide, polynucleotide, or the equivalent of either which will be specifically recognized by certain antibodies which, when raised to the protein, polypeptide or polynucleotide according to the invention, interferes with the immediate physical interaction between pathogen and mammalian host.

As used herein, the term “essential”, when used in connection with a gene or gene product, means that the host cannot survive without, or is significantly growth compromised, in the absence or depletion of functional product. An “essential gene” is thus one that encodes a product that is beneficial, or preferably necessary, for cellular growth in vitro in a medium appropriate for growth of a strain having a wild-type allele corresponding to the particular gene in question. Therefore, if an essential gene is inactivated or inhibited, that cell will grow significantly more slowly than a wild-type strain or even not at all. Preferably, growth of a strain in which such a gene has been inactivated will be less than 20%, more preferably less than 10%, most preferably less than 5% of the growth rate of the wild-type, or the rate will be zero, in the growth medium. Preferably, in the absence of activity provided by a product of the gene, the cell will not grow at all or will be non-viable, at least under culture conditions similar to normal in vivo growth conditions. For example, absence of the biological activity of certain enzymes involved in bacterial cell wall synthesis can result in the lysis of cells under normal osmotic conditions, even though protoplasts can be maintained under controlled osmotic conditions. Preferably, but not necessarily, if such a gene is inhibited, e.g., with an antibacterial agent or a phage product, the growth rate of the inhibited bacteria will be less than 50%, more preferably less than 30%, still more preferably less than 20%, and most preferably less than 10% of the growth rate of the uninhibited bacteria. As recognized by those skilled in the art, the degree of growth inhibition will generally depend upon the concentration of the inhibitory agent. In the context of the invention, essential genes are generally the preferred targets of antimicrobial agents. Essential genes can encode “target” molecules directly or can encode a product involved in the production, modification, or maintenance of a target molecule.

As used herein, “target” refers to a biomolecule or complex of biomolecules that can be acted on by an exogenous agent or compound, thereby modulating, preferably inhibiting, growth or viability of a bacterial cell. A target may be a nucleic acid sequence or molecule, or a polypeptide or a region of a polypeptide.

As used herein, the term “signal that is generated by interaction of a S. aureus polypeptide comprising the amino acid sequence of SEQ ID NO: 2, or fragments thereof to a 44AHJD ORF 25, Twort ORF 168 or G1 ORF 240 or fragment thereof” or the like refers to the measurable indicator of polypeptide comprising the amino acid sequence of SEQ ID NO: 2, or fragments thereof and 44AHJD ORF 25, Twort ORF 168 or G1 ORF 240 or fragment thereof interaction in a binding assay. As used herein, the term “signal that is generated by activation or inhibition of a S. aureus polypeptide comprising the amino acid sequence of SEQ ID NO: 2, or fragments thereof” refers to the measurable indicator of polypeptide comprising the amino acid sequence of SEQ ID NO: 2, or fragments thererof, activity in an assay of STAAU_R2 activity. For example, the signal may include, but is not limited to (i) a signal resulting from binding of 44AHJD ORF 25, Twort ORF 168 or G1 ORF 240 to a STAAU_R2 polypeptide, including a fluorescence signal (fluorescence resonance energy transfer assay; fluorescence polarization assay), spectrophotometer absorbance measurement of a colourimetric signal (phage display ELISA), mass change measurement (surface plasmon resonance analysis), or a viability measurement on selective medium (yeast two-hybrid analysis); or (ii) a reduction of a radiolabeled signal (DNA synthesis assay).

As used herein, the term “standard”, used in reference to polypeptide activity, means the amount of activity observed or detected (directly or indirectly) in a given assay performed in the absence of a candidate compound. A “standard” serves as a reference to determine the effect, positive or negative, of a candidate compound on polypeptide activity.

As used herein, the term “increase in activity” refers to an enhanced level of measurable activity of a polypeptide in a given assay in the presence of a candidate compound relative to the measurable level of activity in the absence of a candidate compound. Activity is considered increased according to the invention if it is at least 10% greater, 20% greater, 50% greater, 75% greater, 100% greater or more, up to 2-fold, 5-fold, 10-fold, 20-fold, 50-fold, 100-fold or more than in the absence of a candidate compound.

As used herein, the term “decrease in activity” refers to a reduced level of measurable activity of a polypeptide in a given assay in the presence of a candidate compound relative to the measurable level of activity in the absence of a candidate compound. Activity is considered decreased according to the invention if it is at least 10% less, preferably 15% less, 20% less, 50% less, 75% less, or even 100% less (i.e., no activity) than that observed in the absence of a candidate compound.

As used herein, the term “conditions that permit their interaction”, when used in reference to a S. aureus polypeptide comprising the amino acid sequence of SEQ ID NO: 2, or fragments thereof, and a candidate compound means that the two entities are placed together, whether both in solution or with one immobilized or restricted in some way and the other in solution, wherein the parameters (e.g., salt, detergent, protein or candidate compound concentration, temperature, and redox potential, among others) of the solution are such that the S. aureus polypeptide comprising the amino acid sequence of SEQ ID NO: 2, or fragments thereof, and the candidate compound may physically associate. Conditions that permit protein:candidate interaction include, for example, the conditions described herein for FRET, fluorescent polarization, Surface Plasmon Resonance and Phage display assays.

As used herein, the term “detectable change in a measurable parameter of STAAU_R2” refers to an alteration in a quantifiable characteristic of a S. aureus STAAU_R2 polypeptide.

As used herein, the term “agonist” refers to an agent or compound that enhances or increases the activity of a S. aureus STAAU_R2 polypeptide or polynucleotide. An agonist may be directly active on a S. aureus STAAU_R2 polypeptide or polynucleotide, or it may be active on one or more constituents in a pathway that leads to enhanced or increased activity of a S. aureus STAAU_R2 polypeptide or polynucleotide.

As used herein, the term “antagonist” refers to an agent or compound that reduces or decreases the activity of a S. aureus STAAU_R2 polypeptide or polynucleotide. An antagonist may be directly active on a S. aureus STAAU_R2 polypeptide or polynucleotide, or it may be active on one or more constituents in a pathway that leads to reduced or decreased activity of a S. aureus STAAU_R2 polypeptide or polynucleotide.

As used herein, the term “antibacterial agent” or “antibacterial compound” refers to an agent or compound that has a bacteriocidal or bacteriostatic effect on one or more bacterial strains, preferably such an agent or compound is bacteriocidal or bacteriostatic on at least S. aureus.

As used herein, the term “synthesizing” refers to a process of chemically synthesizing a compound.

As used in the context of treating a bacterial infection a “therapeutically effective amount”, “pharmaceutically effective amount” or “amount sufficient to provide a therapeutic effect” indicates an amount of an antibacterial agent which has a therapeutic effect. This generally refers to the inhibition, to some extent, of the normal cellular functioning of bacterial cells required for continued bacterial infection. Further, as used herein, a therapeutically effective amount means an amount of an antibacterial agent that produces the desired therapeutic effect as judged for example by clinical trial results and/or animal models. This amount can be routinely determined by one skilled in the art and will vary depending on several factors, such as the particular bacterial strain involved and the particular antibacterial agent used. In the same context, an “amount sufficient to reduce adhesion” of a bacterium to a tissue or tissue surface indicates an amount of an antibacterial agent that is effective for prophylactically preventing or reducing the extent of bacterial infection of the given tissue or tissue surface.

As used in the context of treating a bacterial infection, contacting or administering the antimicrobial agent ‘in combination with existing antimicrobial agents’ refer to a concurrent contacting or administration of the active compound with antibiotics to provide a bactericidal or growth inhibitory effects beyond the individual bactericidal or growth inhibitory effects of the active compound or the antibiotic. Existing antibiotic refers for example to the group consisting of penicillins, cephalosporins, imipenem, monobactams, aminoglycosides, tetracyclines, sulfonamides, trimethoprim/sulfonamide, fluoroquinolones, macrolides, vancomycin, polymyxins, chloramphenicol and lincosamides.

As used herein, a “tissue” refers to an aggregation of cells of one or more cell types which together perform one or more specific functions in an organism. As used herein, a “tissue surface” refers to that portion of a tissue that forms a boundary between a given tissue and other tissues or the surroundings of the tissue. A tissue surface may refer to an external surface of an animal, for example the skin or cornea, or, alternatively, the term may refer to a surface that is either internal, for example, the lining of the gut, or to a surface that is exposed to the outside surroundings of the animal only as the result of an injury or a surgical procedure.

As used herein, the term “measuring the binding of a candidate compound” refers to the use of an assay permitting the quantitation of the amount of a candidate compound physically associated with a S. aureus STAAU_R2 polypeptide, fragment or variant thereof.

A “candidate compound” as used herein, is any compound with a potential to modulate the expression or activity of a S. aureus STAAU_R2 polypeptide.

As used herein, the term “directly or indirectly detectably labeled” refers to the attachment of a moiety to a candidate compound that renders the candidate compound either directly detectable (e.g., an isotope or a fluorophore) or indirectly detectable (e.g., an enzyme activity, allowing detection in the presence of an appropriate substrate, or a specific antigen or other marker allowing detection by addition of an antibody or other specific indicator).

A “method of screening” refers to a method for evaluating a relevant activity or property of a large plurality of compounds, rather than just one or a few compounds. For example, a method of screening can be used to conveniently test at least 100, more preferably at least 1000, still more preferably at least 10,000, and most preferably at least 100,000 different compounds, or even more. In a particular embodiment, the method is amenable to automated, cost-effective high throughput screening on libraries of compounds for lead development.

In a related aspect or in preferred embodiments, the invention provides a method of screening for potential antibacterial agents by determining whether any of a plurality of compounds, preferably a plurality of small molecules, is active on STAAU_R2. Preferred embodiments include those described for the above aspect, including embodiments which involve determining whether one or more test compounds bind to or reduce the level of activity of a bacterial target, and embodiments which utilize a plurality of different targets as described above.

The term “compounds” preferably includes, but is not limited to, small organic molecules, peptides, polypeptides and antibodies that bind to a polynucleotide and/or polypeptide of the invention, such as for example inhibitory ORF gene product or target thereof, and thereby inhibit, extinguish or enhance its activity or expression. Potential compounds may be small organic molecules, a peptide, a polypeptide such as a closely related protein or antibody that binds the same site(s) on a binding molecule, such as a bacteriophage gene product, thereby preventing bacteriophage gene product from binding to STAAU_R2 polypeptides.

The term “compounds” also potentially includes small molecules that bind to and occupy the binding site of a polypeptide, thereby preventing binding to cellular binding molecules, such that normal biological activity is prevented. Examples of small molecules include but are not limited to small organic molecules, peptides or peptide-like molecules. Preferred potential compounds include compounds related to and variants of inhibitory ORF encoded by a bacteriophage and of STAAU_R2 and any homologues and/or peptidomimetics and/or fragments thereof. Other examples of potential polypeptide antagonists include antibodies or, in some cases, oligonucleotides or proteins which are closely related to the ligands, substrates, receptors, enzymes, etc., as the case may be, of the polypeptide, e.g., a fragment of the ligands, substrates, receptors, enzymes, etc.; or small molecules which bind to the polypeptide of the present invention but do not elicit a response, so that the activity of the polypeptide is prevented. Other potential compounds include antisense molecules (see Okano, 1991 J. Neurochem. 56, 560; see also “Oligodeoxynucleotides as Antisense Inhibitors of Gene Expression”, CRC Press, Boca Raton, Fla. (1988), for a description of these molecules).

As used herein, the term “library” refers to a collection of 100 compounds, preferably of 1000, still more preferably 5000, still more preferably 10,000 or more, and most preferably of 50,000 or more compounds.

As used herein, the term “small molecule” refers to compounds having molecular mass of less than 3000 Daltons, preferably less than 2000 or 1500, still more preferably less than 1000, and most preferably less than 600 Daltons. Preferably but not necessarily, a small molecule is not an oligopeptide.

As used herein, the term “mimetic” refers to a compound that can be natural, synthetic, or chimeric and is structurally and functionally related to a reference compound. In terms of the present invention, a “peptidomimetic,” for example, is a non-peptide compound that mimics the activity-related aspects of the 3-dimensional structure of a peptide or polypeptide, for example a compound that mimics the structure of a peptide or active portion of a phage- or bacterial ORF-encoded polypeptide.

As used herein, the term “bacteriophage inhibitor protein” refers to a protein encoded by a bacteriophage nucleic acid sequence, which inhibits bacterial function in a host bacterium. Thus, it is a bacteria-inhibiting phage product. The term “bacteriophage inhibitor protein” encompasses a fragment, derivative, or active portion of a bacteriophage inhibitor protein.

In more than one embodiment of the above assay methods of the present invention, it may be desirable to immobilize either STAAU_R2 or its target molecule or ligand to facilitate separation of complexed from uncomplexed forms of one or both of the proteins or polypeptides, as well as to accommodate automation of the assay. Binding of a test compound to a STAAU_R2 protein or interaction of a STAAU_R2 protein with a target molecule or ligand in the presence and absence of a candidate compound, can be accomplished in any vessel suitable for containing the reactants. Examples of such vessels include microtitre plates, test tubes and micro-centrifuge tubes. In one embodiment a fusion protein can be provided which adds a domain that allows one or both of the proteins to be bound to a matrix. For example, glutathione-S-transferase/STAAU_R2 fusion proteins or glutathione-S-transferase/target fusion proteins (e.g. glutathione-S-transferase/Twort ORF168) can be adsorbed onto glutathione sepharose beads (Sigma Chemical, St. Louis, Mo.) or glutathione derivatized microtitre plates, which are then combined with the test compound or the test compound and either the non-adsorbed target protein or STAAU_R2 protein and the mixture incubated under conditions conducive to complex formation (e.g. at physiological conditions for salt and pH). Following incubation the beads or microtitre plate wells are washed to remove any unbound components, the matrix immobilized in the case of beads, complex determined either directly or indirectly, for example, as described above. Alternatively, the complexes can be dissociated from the matrix, and the level of STAAU_R2 binding or activity determined using standard techniques.

Other techniques for immobilizing proteins on matrices (and well-known in the art) can also be used in the screening assays of the invention. For example, either a STAAU_R2 protein or a STAAU_R2 target molecule or ligand can be immobilized utilizing conjugation of biotin and streptavidin. Biotinylated STAAU_R2 protein or target molecules or ligand can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with CI-MPR protein, target molecules or ligand but which do not interfere with binding of the STAAU_R2 protein to its target molecule or ligand can be derivatized to the wells of the plate, and unbound target or STAAU_R2 protein trapped in the wells by antibody conjugation. Methods for detecting such complexes, in addition to those described above for the GST-immobilized complexes, include immunodetection of complexes using antibodies reactive with the STAAU_R2 protein or target molecule, as well as enzyme-linked assays which rely on detecting an enzymatic activity associated with the STAAU_R2 protein or target molecule and in particular with Twort ORF168, 44AHJD ORF25, and G1 ORF 240.

As used herein, the term “active portion” refers to an epitope, a catalytic or regulatory domain, or a fragment of a bacteriophage inhibitor protein that is responsible for, or a significant factor in, bacterial target inhibition. The active portion preferably may be removed from its contiguous sequences and, in isolation, still effect inhibition.

As used herein, the term “treating a bacterial infection” refers to a process whereby the growth and/or metabolic activity of a bacterium or bacterial population in a host, preferably a mammal, more preferably a human, is inhibited or ablated.

As used herein, the term “bacterium” refers to a single bacterial strain and includes a single cell and a plurality or population of cells of that strain unless clearly indicated to the contrary. In reference to bacteria or bacteriophage, the term “strain” refers to bacteria or phage having a particular genetic content. The genetic content includes genomic content as well as recombinant vectors. Thus, for example, two otherwise identical bacterial cells would represent different strains if each contained a vector, e.g., a plasmid, with different inserts.

As used herein, the term “diagnosing” refers to the identification of an organism or strain of an organism responsible for a bacterial infection.

As used herein, the term “infection with Staphylococcus aureus ” refers to the presence, growth or proliferation of cells of a S. aureus strain within, or on a surface of, an animal, such as a mammal, preferably a human.

As used herein, the term “bacteriophage 44AHJD ORF 25-encoded polypeptide” refers to a polypeptide encoded by SEQ ID NO: 3 or to a fragment or derivative thereof encompassing an active portion of a bacteriophage 44AHJD ORF 25-encoded polypeptide of sequence disclosed in SEQ ID NO: 4.

As used herein, the term “bacteriophage Twort ORF168-encoded polypeptide” refers to a polypeptide encoded by SEQ ID NO: 5 or to a fragment or derivative thereof encompassing an active portion of a bacteriophage Twort ORF168-encoded polypeptide of sequence disclosed in SEQ ID NO: 6.

As used herein, the term “bacteriophage G1 ORF 240-encoded polypeptide” refers to a polypeptide encoded by SEQ ID NO: 9 or to a fragment or derivative thereof encompassing an active portion of a bacteriophage G1 ORF 240-encoded polypeptide of sequence disclosed in SEQ ID NO: 10.

As used herein, the term “polypeptide complex” refers to a combination of two or more polypeptides in a physical association with each other. It is preferred that such a physical association be required for some aspect of the activity of one or more of the polypeptides in such a polypeptide complex.

As used herein, the term “physical association” refers to an interaction between two moieties involving contact between the two moieties.

As used herein, the term “bodily material(s)” means any material derived from an individual or from an organism infecting, infesting or inhabiting an individual, including but not limited to, cells, tissues and waste, such as, bone, blood, serum, cerebrospinal fluid, semen, saliva, muscle, cartilage, organ tissue, skin, urine, stool or autopsy materials.

As used herein, the term “disease(s)” means any disease caused by or related to infection by a bacterium, including, for example, otitis media, conjunctivitis, pneumonia, bacteremia, meningitis, sinusitis, pleural empyema and endocarditis, and most particularly meningitis, such as for example infection of cerebrospinal fluid.

As used herein, the term “fusion protein(s)” refers to a protein encoded by a gene comprising amino acid coding sequences from two or more separate proteins fused in frame such that the protein comprises fused amino acid sequences from the separate proteins.

As used herein, the term “host cell(s)” is a cell which has been transformed or transfected, or is capable of transformation or transfection by an exogenous polynucleotide sequence.

As used herein, the term “immunologically equivalent derivative(s)” encompasses a polypeptide, polynucleotide, or the equivalent of either which when used in a suitable formulation to raise antibodies in a vertebrate, results in antibodies that act to interfere with the immediate physical interaction between pathogen and mammalian host.

As used herein, the term “immunospecific” means that characteristic of an antibody whereby it possesses substantially greater affinity for the polypeptides of the invention or the polynucleotides of the invention than its affinity for other related polypeptides or polynucleotides respectively, particularly those polypeptides and polynucleotides in the prior art.

As used herein, the term “individual(s)” means a multicellular eukaryote, including, but not limited to a metazoan, a mammal, an ovid, a bovid, a simian, a primate, and a human.

As used herein, the term “Organism(s)” means a (i) prokaryote, including but not limited to, a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israeli, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii and Chlamydia trachomitis, (ii) an archaeon, including but not limited to Archaebacter, and (iii) a unicellular or filamenous eukaryote, including but not limited to, a protozoan, a fungus, a member of the genus Saccharomyces, Kluveromyces, or Candida, and a member of the species Saccharomyces ceriviseae, Kluveromyces lactis, or Candida albicans.

As used herein, the term “recombinant expression system(s)” refers to a system in which vectors comprising sequences encoding polypeptides of the invention or portions thereof, or polynucleotides of the invention are introduced or transformed into a host cell or host cell lysate for the production of the polynucleotides and polypeptides of the invention.

As used herein, the term “artificially synthesized” when used in reference to a peptide, polypeptide or polynucleotide means that the amino acid or nucleotide subunits were chemically joined in vitro without the use of cells or polymerizing enzymes. The chemistry of polynucleotide and peptide synthesis is well known in the art.

In addition to the standard single and triple letter representations for amino acids, the term “X” or “Xaa” may also be used in describing certain polypeptides of the invention. “X” and “Xaa” mean that any of the twenty naturally occurring amino acids may appear at such a designated position in the polypeptide sequence.

As used herein, the term “specifically binding” in the context of the interaction of two polypeptides means that the two polypeptides physically interact via discrete regions or domains on the polypeptides, wherein the interaction is dependent upon the amino acid sequences of the interacting domains. Generally, the equilibrium binding concentration of a polypeptide that specifically binds another is in the range of about 1 mM or lower, more preferably 1 uM or lower, preferably 100 nM or lower, 10 nM or lower, 1 nM or lower, 100 pM or lower, and even 10 pM or lower.

As used herein, the term “decrease in the binding” refers to a drop in the signal that is generated by the physical association between two polypeptides under one set of conditions relative to the signal under another set of reference conditions. The signal is decreased if it is at least 10% lower than the level under reference conditions, and preferably 20%, 40%, 50%, 75%, 90%, 95% or even as much as 100% lower (i.e., no detectable interaction).

As used herein, the term “detectable marker”, when used in the context of a yeast two-hybrid assay, refers to a polypeptide that confers a trait upon a cell expressing that polypeptide that signals the presence or amount of that polypeptide expressed. Detectable markers are encoded on plasmids that may exist episomally or may be integrated into the genome of a host cell. Detectable markers include, but are not limited to, polypeptides encoding enzymes allowing colorimetric or fluorescent detection (e.g., E. coli LacZ, which catalyzes the conversion of the substrate analog X-gal to generate a blue color), polypeptides encoding enzymes conferring antibiotic resistance, and polypeptides encoding enzymes conferring the ability of a yeast strain to grow on medium lacking a given component (i.e., critical for the relief of auxotrophy).

As used herein, the term “results in the expression of a detectable marker” means that the interaction of factors necessary to permit the expression of a detectable marker (e.g., two-hybrid transactivation domain and DNA binding domain fusion proteins) causes the transactivation and translation of detectable levels of a detectable marker. A “detectable level” is that level of expression that can be differentiated from background expression occurring in the substantial absence of one or more factors or conditions necessary for marker expression. Detectable levels will vary depending upon the nature of the detectable marker, but will generally consist of levels at least about 10% or more greater than the background level of a given marker.

As used herein, the term “decrease in the expression” refers to a drop in the expression of a detectable marker under one set of conditions relative to the expression under another set of reference conditions. The expression of a detectable marker is decreased if it is at least 10% lower than the level under reference conditions, and preferably 20%, 40%, 50%, 75%, 90%, 95% or even as much as 100% lower (i.e., not expressed).

Identification of the S. aureus STAAU_R2 Sequence

The methodology used to identify the STAAU_R2 polypeptide is described in detail in U.S. Provisional Patent Application No. 60/110,992, filed Dec. 3,1998, and PCT International Application WO1999/IB99/02040, filed Dec. 3, 1999.

A S. aureus polypeptide that specifically bound the bacterial growth inhibitory 44AHJD ORF 25, Twort ORF 168 or G1 ORF 240 proteins was isolated. Briefly, the three inhibitory ORF proteins were used separately as a ligand in an affinity chromatography binding step with S. aureus protein extract.

The selected S. aureus interacting polypeptide, herein referred as STAAU_R2, was purified and further analyzed by tryptic digestion and mass spectrometry using MALDI-ToF technology [Qin, J., et al. (1997) Anal. Chem. 69, 3995-4001]. Computational analysis of the mass spectrum obtained identified the corresponding ORF as the S. aureus DNA polymerase III, beta subunit. The interaction between Twort ORF 168 and the STAAU_R2, was confirmed in a yeast two-hybrid assay. The interaction between 44AHJD ORF 25 and the candidate target protein was confirmed by surface plasmon resonance.

Function of the DNA Polymerase III, Beta (β) Subunit

DNA polymerase III holoenzyme is an essential component of bacterial DNA replication machinery. The holoenzyme contains several different polypeptide chains. Type III polymerases are exemplified by the replicase of the Gram-negative bacterium Escherichia coli, in which there are three separate components: a sliding clamp protein, a clamp loader complex and the DNA polymerase itself [Kelman et al. 1995, Annu. Rev. Biochem. 64: 171-200]. The clamp loader is a multiprotein complex which uses ATP to assemble the sliding clamp around DNA. The DNA polymerase then binds to the sliding clamp which tethers the polymerase to the DNA. The DNA polymerase III beta (β) subunit is a homodimer and forms the ring shaped sliding clamp associated with DNA.

Bacillus subtilis and Streptococcus pyogenes are the best characterized Gram-positive bacteria with respect to DNA replication [Barnes et al. 1995, Methods in Enzy. 262: 35-42; Bruck and O'Donnell 2000, J. Biol. Chem. 275: 28971-28983]. Many genes involved in Bacillus subtilis DNA replication have been identified through the isolation of ts mutants. Studies in B. subtilis have identified a polymerase that appears to be involved in chromosome replication and is termed Pol III. The polC gene encodes Pol III, a large polypeptide likely corresponding to the alpha and epsilon subunits of the E. coli enzyme. In in vitro reconstituted assays, five Streptococcus pyogenes proteins were shown to coordinate their actions to achieve rapid and processive DNA synthesis: the PolC DNA polymerase, τ, δ, δ′ and β [Bruck and O'Donnell 2000, J. Biol. Chem. 275: 28971-28983].

S. aureus has a gene encoding a protein with 30% homology to the β of the E. coli enzyme. The S. aureus gene corresponding to the E. coli β subunit is dnaN. S. aureus dnaN has been described in two PCT Applications (WO 9937661; and WO 0109164).

The E. coli β subunit has been shown to interact with a variety of proteins including DNA polymerase III α and δ subunits, DNA polymerase I, DNA polymerase II, DNA polymerase V, DNA ligase and MutS [Jeruzalmi et al. 2001, Cell, 106: 417-428; Lopez de Saro and O'Donnell 2001, PNAS, 98: 8376-8380]. Surprisingly, despite the demonstration of protein-protein interactions of the β subunit with a variety of proteins and despite evidence that these interactions within the replisome are critical to obtaining efficient chromosome replication in vitro [Turner et al. 1999, The EMBO J. 18: 771-783; Jeruzalmi et al. 2001, Cell, 106: 417-428; Bruck and O'Donnell 2000, J. Biol. Chem. 275: 28971-28983], there are currently no available drugs directed against the sliding clamp.

The demonstration that bacteriophage have adapted to inhibiting a host bacterium by acting on a particular cellular component or target provides a strong indication that that component is an appropriate target for developing and using antibacterial agents, e.g. in therapeutic treatments. The present invention provides additional guidance over mere identification of bacterial essential genes, as the present invention also provides an indication of accessibility of the target to an inhibitor, and an indication that the target is sufficiently stable over time (e.g., not subject to high rates of mutation) as phage acting on that target were able to develop and persist. Thus, the present invention identifies STAAU_R2 as an appropriate target for development of antibacterial agents.

Identification of the Surface of Interaction on STAAU_R2

This invention relates, in part, to a specific interaction between a growth-inhibitory protein encoded by the S. aureus bacteriophage genome and an essential S. aureus protein. In one embodiment, this interaction forms the basis for drug screening assays. More specifically, the invention relates to the interacting regions of the protein encoded by the S. aureus STAAU_R2 and the S. aureus bacteriophage 44AHJD ORF 25, Twort ORF 168, G1 ORF 240 proteins, forming the basis for screening assays. The invention provides a method for the identification of 44AHJD ORF 25, Twort ORF 168 (e.g. with SEQ ID NO: 8) or G1 ORF 240 and, more preferably, STAAU_R2 polypeptide fragments which are involved in the interaction between STAAU_R2 and 44AHJD ORF 25, Twort ORF 168, G1 ORF 240.

Several approaches and techniques known to those skilled in the art can be used to identify and to characterize interacting fragments of STAAU_R2, 44AHJD ORF 25, Twort ORF 168, G1 ORF 240. These fragments may include, for example, truncation polypeptides having a portion of an amino acid sequence of any of the two proteins, or variants thereof, such as a continuous series of residues that includes an amino- and/or carboxyl-terminal amino acid sequence.

Fragments of STAAU_R2,44AHJD ORF 25, Twort ORF 168, G1 ORF 240_can be cloned by genetic recombinant technology and tested for interaction using a yeast two-hybrid assay as exemplified below.

Partial proteolysis of proteins in solution is one method to delineate the domain boundaries in multi-domain proteins. By subjecting proteins to limited digestion, the most accessible cleavage sites are preferentially hydrolyzed. These cleavage sites preferentially reside in less structured regions which include loops and highly mobile areas typical of the joining amino acids between highly structures domains. Purified STAAU_R2, 44AHJD ORF 25, Twort ORF 168, G1 ORF 240_proteins can be subjected to partial proteolysis. The proteolysis can be performed with low concentrations of proteases (trypsin, chymotrypsin, endoproteinase Glu-C, and Asp-N) with STAAU_R2, 44AHJD ORF 25, Twort ORF 168, G1 ORF 240 in solution, resulting in the generation of defined proteolytic products as observed by SDS-PAGE. An acceptable concentration and reaction time is defined by the near complete conversion of the full-length protein to stable proteolytic products. The proteolytic products are then subjected to affinity chromatography containing the appropriated partner of interaction (44AHJD ORF 25, Twort ORF 168; G1 ORF 240_or STAAU_R2 purified proteins) to determine a protein sub-region able to interact. Interacting domains are identified by mass spectrometry to determine both the intact fragment mass and the completely digested with trypsin (by in-gel digestion) to better determine the amino acid residues contained within the partial proteolytic fragment. Using both sets of data, the amino acid sequence of the partial proteolytic fragment can be precisely determined.

Another approach is based on peptide screening using different portions of 44AHJD ORF 25, Twort ORF 168, G1 ORF 240 or STAAU_R2 to identify minimal peptides from each polypeptide that are able to disrupt the interaction between the two proteins. It is assumed that fragments able to prevent interaction between STAAU_R2 and 44AHJD ORF 25, Twort ORF 168, or G1 ORF 240 correspond to domains of interaction located on either of the two interacting proteins. The different peptide fragments can be screened as competitors of interaction in protein: protein binding assays such as the ones described below. Fine mapping of interaction site(s) within a protein can be performed by an extensive screen of small overlapping fragments or peptides spanning the entire amino acid sequence of the protein.

Suitable STAAU_R2, Twort ORF 168, 44AHJD ORF 25, G1 ORF 240—derived amino acid fragments representative of the complete sequence of both proteins can be chemical synthesis. For instance, in the multipin approach, peptides are simultaneously synthesis by the assembly of small quantities of peptides (ca. 50 nmol) on plastic pins derivatized with an ester linker based on glycolate and 4-(hydroxymethyl) benzoate (Maeji 1991 Pept Res, 4:142-6).

S. aureus STAAU_R2 Polypeptides

In one aspect of the invention there are provided polypeptides of S. aureus referred to herein as “STAAU_R2” and “STAAU_R2 polypeptides” as well as biologically, diagnostically, prophylactically, clinically or therapeutically useful variants thereof, and compositions comprising the same.

Among the particularly preferred embodiments of the invention are variants of S. aureus STAAU_R2 polypeptides encoded by naturally occurring alleles of the STAAU_R2 gene. The present invention provides for an isolated polypeptide which comprises or consists of: (a) an amino acid sequence which has at least 40% identity, preferably at least 50% identity, preferably at least 80% identity, more preferably at least 90%, yet more preferably at least 95%, most preferably at least 97-99%, or exact identity, over the entire length of SEQ ID NO: 2; or b) an amino acid sequence that has at least 60% similarity, at least 70% similarity, at least 80% similarity, at least 90% similarity, at least 95% similarity, at least 97-99% similarity or even 100% similarity over the entire length of SEQ ID NO: 2.

The polypeptides of the invention include a polypeptide of FIG. 1 (SEQ ID NO: 2) (in particular the mature polypeptide) as well as polypeptides and fragments, particularly those which have a biological activity of STAAU_R2, and also those which have at least 40% identity over 50 or more amino acids to a polypeptide of SEQ ID NO: 2 or the relevant portion, preferably at least 60%, 70%, or 80% identity over 50 or more amino acids to a polypeptide of SEQ ID NO: 2, more preferably at least 90% identity over 50 or more amino acids to a polypeptide of SEQ ID NO: 2 and still more preferably at least 95% identity over 50 or more amino acids to a polypeptide of SEQ ID NO: 2 and yet still more preferably at least 99% identity over 50 or more amino acids to a polypeptide of SEQ ID NO: 2.

The polypeptides of the invention also include a polypeptide or protein fragment that has at least 60%, 70%, 80% or 90% similarity, 95% similarity or even 97-99% similarity over 50 or more amino acids to a polypeptide of SEQ ID NO: 2.

It is most preferred that a polypeptide of the invention is derived from S. aureus, however, it may be obtained from other organisms of the same taxonomic genus. A polypeptide of the invention may also be obtained, for example, from organisms of the same taxonomic family or order.

Fragments of STAAU_R2 also are included in the invention. These fragments may include, for example, truncation polypeptides having a portion of an amino acid sequence of FIG. 1 (SEQ ID NO: 2), or variants thereof, such as a continuous series of residues that includes an amino- and/or carboxyl-terminal amino acid sequence. Degradation forms of the polypeptides of the invention produced by or in a host cell, particularly S. aureus, are also preferred. Further preferred are fragments characterized by structural or functional attributes such as fragments that comprise alpha-helix and alpha-helix-forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Fragments of STAAU_R2 may be expressed as fusion proteins with other proteins or protein fragments.

Preferred fragments also include an isolated polypeptide comprising an amino acid sequence having at least 10, 20, 30, 40, 50, or 100 or more contiguous amino acids from the amino acid sequence of SEQ ID NO: 2.

Also preferred are biologically “active” fragments which are those fragments that mediate activities of S. aureus STAAU_R2, including those with a similar activity or an improved activity, or with a decreased undesirable activity. Also included are those fragments that are antigenic or immunogenic in an animal, especially in a human. Particularly preferred are fragments comprising domains that confer a function essential for viability of S. aureus.

Fragments of the polypeptides of the invention may be employed for producing the corresponding full-length polypeptide by peptide synthesis; therefore, these variants may be employed as intermediates for producing the full-length polypeptides of the invention.

S. aureus Polynucleotides

It is an object of the invention to provide polynucleotides that encode STAAU_R2 polypeptides, particularly polynucleotides that encode the polypeptide herein designated S. aureus STAAU_R2.

In one aspect of the invention, a polynucleotide is provided that comprises a region encoding a S. aureus STAAU_R2 polypeptide, the polynucleotide comprising a sequence set out in SEQ ID NO: 1. Such a polynucleotide encodes a full length STAAU_R2 gene, or a variant thereof. It is contemplated that this full-length gene is essential to the growth and/or survival of an organism which possesses it, such as S. aureus.

As a further aspect of the invention there are provided isolated nucleic acid molecules encoding and/or expressing a fragment of a full-length STAAU_R2 polypeptide, particularly a S. aureus STAAU_R2 polypeptide or a variant thereof. Further embodiments of the invention include biologically, diagnostically, prophylactically, clinically or therapeutically useful polynucleotides, polypeptides, variants thereof, and compositions comprising same.

A polynucleotide of the invention is obtained using S. aureus cells as starting material, the nucleotide sequence information disclosed in SEQ ID NO: 1, and standard cloning and screening methods, such as those for cloning and sequencing chromosomal DNA fragments from bacteria. For example, to obtain a polynucleotide sequence of the invention, such as the polynucleotide sequence disclosed as in SEQ ID NO: 1, a library of clones of chromosomal DNA of S. aureus in E. coli or another suitable host is probed with a radiolabeled oligonucleotide, preferably a 17-mer or longer, derived from a partial sequence. Clones carrying DNA identical to that of the probe can be distinguished using stringent hybridization conditions. As herein used, the terms “stringent conditions” and “stringent hybridization conditions” mean hybridization occurring only if there is at least 95% and preferably at least 97% identity between the sequences. A specific example of stringent hybridization conditions is of an overnight incubation of a hybridization support (e.g., a nylon or nitrocellulose membrane) at 42° C. in a solution comprising: 1×10⁶ cpm/ml labeled probe, 50% formamide, 5×SSC (150 mM NaCl, 15 mM trisodium citrate), 50 mM sodium phosphate (pH 7.6), 5× Denhardt's solution, 10% dextran sulfate, and 20 micrograms/ml of denatured, sheared salmon sperm DNA, followed by washing the hybridization support in 0.1×SSC at 65° C. Hybridization and wash conditions are well known to those skilled in the art and are exemplified in Sambrook, et al., Molecular Cloning: A Laboratory Manual, Second Edition, Cold Spring Harbor, N.Y., (1989), particularly Chapter 11 therein. Solution hybridization may also be used with the polynucleotide sequences provided by the invention. By sequencing the individual clones thus identified by hybridization, it is possible to confirm the identity of the clone.

Alternatively, an amplification process can be utilized to isolate the poylnucleotide. In this approach, the sequence disclosed as SEQ ID NO: 1 is targeted by two oligonucleotides, one identical to a sequence on the coding DNA strand at or upstream of the ATG initiation codon and the other which anneals to the opposite strand at or downstream of the stop codon. Priming from these oligonucleotides in a polymerase chain reaction yields a full-length gene coding sequence. Such suitable techniques are described by Sambrook et al., Molecular Cloning: A Laboratory Manual, 2^{nd} Edition, Cold Spring Harbor Laboratory Press, Cold Spring Harbor, N.Y. (1989).

In a further aspect, the present invention provides for an isolated polynucleotide comprising or consisting of: (a) a polynucleotide sequence which has at least 60% identity, preferably at least 70% identity, more preferably at least 80% identity, more preferably at least 90% identity, yet more preferably at least 95%, most preferably at least 97-99% or exact identity, to that of SEQ ID NO: 1 over the entire length of SEQ ID NO: 1; (b) a polynucleotide sequence encoding a polypeptide which has at least 50% identity, preferably at least 60% identity, more preferably at least 70% identity, more preferably at least 80% identity, more preferably at least 90%, yet more preferably at least 95%, most preferably at least 97-99% or exact identity to SEQ ID NO: 2 over the entire length of SEQ ID NO: 2; or the complement of a sequence of (a) or (b) above.

The invention provides a polynucleotide sequence identical over its entire length to the coding sequence of SEQ ID NO: 1. Also provided by the invention is a coding sequence for a mature polypeptide or a fragment thereof by itself as well as a coding sequence for a mature polypeptide or a fragment in reading frame with another coding sequence, such as a sequence encoding a leader or secretory sequence, a pre-, or pro-, or prepro-protein sequence. The polynucleotide of the invention may also contain at least one non-coding sequence, including for example, but not limited to at least one non-coding 5′ and 3′ sequence, such as the transcribed but non-translated sequences, termination signals (such as rho-dependent and rho-independent termination signals), ribosome binding sites, Kozak sequences, sequences that stabilize or destabilize mRNAs, introns, and polyadenylation signals. The polynucleotide sequence may also comprise additional coding sequence encoding additional amino acids. For example, a marker sequence that facilitates purification of the fused polypeptide can be encoded. In certain embodiments of the invention, the marker sequence is a hexa-histidine peptide, as provided in the pQE vector (Qiagen, Inc.) and described in Gentz et al., Proc. Natl. Acad. Sci. 86: 821-824 (1989), or an HA peptide tag [Wilson et al., Cell 37: 767 (1984)], both of which may-be useful in purifying polypeptide sequences fused to them. Polynucleotides of the invention also include, but are not limited to, polynucleotides comprising a structural gene and its naturally associated sequences that control gene expression.

While it is most preferred that a polynucleotide of the invention be derived from S. aureus, it may also be obtained from other organisms of the same taxonomic genus. A polynucleotide of the invention may also be obtained, for example, from organisms of the same taxonomic family or order.

Further preferred embodiments are polynucleotides encoding S. aureus STAAU_R2 variants that have the amino acid sequence of S. aureus STAAU_R2 polypeptide of SEQ ID NO: 2 in which several, a few, 5 to 10, 1 to 5, 1 to 3, 2, 1 or no amino acid residues are substituted, modified, deleted and/or added, in any combination. Especially preferred among these polynucleotides are those encoding silent nucleotide alterations that do not alter the coding sequence or activities of S. aureus STAAU_R2 polypeptides they encode.

Preferred embodiments are polynucleotides encoding polypeptides that retain substantially the same biological function or activity as the mature polypeptide encoded by a DNA of SEQ ID NO: 1.

In accordance with certain preferred embodiments of this invention there are provided polynucleotides that hybridize, particularly under stringent conditions, to S. aureus STAAU_R2 polynucleotide sequences, such as those polynucleotides in FIG. 1.

The polynucleotides of the invention are useful as hybridization probes for RNA, cDNA and genomic DNA to isolate full-length cDNAs and genomic clones encoding genes that have a high degree of sequence identity to the STAAU_R2 gene. Such probes generally will comprise at least 15 to about 100 residues or base pairs, although such probes will preferably have about 20 to 50 nucleotide residues or base pairs. Particularly preferred probes are about 20 to about 30 nucleotide residues or base pairs in length.

A coding region of a related STAAU_R2 gene from a bacterial species other than S. aureus may be isolated by screening a library using a DNA sequence provided in SEQ ID NO: 1 to synthesize an oligonucleotide probe. A labeled oligonucleotide having a sequence complementary to that of a gene of the invention is then used to screen a library of cDNA, genomic DNA or mRNA to determine to which member(s) of the library the probe hybridizes.

There are several methods available and well known to those skilled in the art to obtain full-length DNAs, or extend short DNAs, for example those based on the method of Rapid Amplification of cDNA Ends (RACE) [see, for example, Frohman, et al., PNAS USA 85: 8998-9002, 1988]. Recent modifications of the technique, exemplified by the MARATHON™ technology (Clontech Laboratories Inc.) for example, have significantly simplified the search for longer cDNAs. In the MARATHON™ technology, cDNAs are prepared from mRNA extracted from a chosen cell and an ‘adaptor’ sequence is ligated onto each end. Nucleic acid amplification by PCR is then carried out to amplify the “missing” 5′ end of the DNA using a combination of gene specific and adaptor specific oligonucleotide primers. The PCR reaction is then repeated using “nested” primers, that is, primers designed to anneal within the amplified product (typically an adaptor-specific primer that anneals further 3′ in the adaptor sequence and a gene-specific primer that anneals further 5′ in the selected gene sequence). The products of this reaction can then be analyzed by DNA sequencing and a full-length DNA constructed either by joining the product directly to the existing DNA to give a complete sequence, or by carrying out a separate full-length PCR using the new sequence information for the design of the 5′ primer.

The polynucleotides and polypeptides of the invention may be employed, for example, as research reagents and materials for discovery of treatments of and diagnostics for diseases, particularly human diseases, as further discussed herein relating to polynucleotide assays.

The polynucleotides of the invention that are oligonucleotides derived from a sequence of SEQ ID NO:1 are useful for the design of PCR primers in reactions to determine whether or not the polynucleotides identified herein in whole or in part are transcribed in bacteria in infected tissue. That is, the polynucleotides of the invention are useful for diagnosis of infection with a bacterial strain carrying those sequences. It is recognized that such sequences also have utility in diagnosis of the stage of infection and type of infection the pathogen has attained.

The invention also provides polynucleotides that encode a polypeptide that is the mature protein plus additional amino or carboxyl-terminal amino acids, or amino acids interior to the mature polypeptide. Such sequences may play a role in processing of a protein from precursor to a mature form, may allow protein transport, may lengthen or shorten protein half-life or may facilitate manipulation of a protein for assay or production, among other things. As generally is the case in vivo, the additional amino acids may be processed away from the mature protein by cellular enzymes.

A precursor protein, having a mature form of the polypeptide fused to one or more prosequences may be an inactive form of the polypeptide. When prosequences are removed such inactive precursors generally are activated. Some or all of the prosequences may be removed before activation. Generally, such precursors are called proproteins.

A polynucleotide of the invention thus may encode a mature protein, a mature protein plus a leader sequence (which may be referred to as a preprotein), a precursor of a mature protein having one or more prosequences that are not the leader sequences of a preprotein, or a preproprotein, which is a precursor to a proprotein, having a leader sequence and one or more prosequences, which generally are removed during processing steps that produce active and mature forms of the polypeptide.

In addition to the standard A, G, C, T/U representations for nucleotides, the term “N” may also be used in describing certain polynucleotides of the invention. “N” means that any of the four DNA or RNA nucleotides may appear at such a designated position in the DNA or RNA sequence, except it is preferred that N is not a nucleotide that when taken in combination with adjacent nucleotide positions, read in the correct reading frame, would have the effect of generating a premature termination codon in such reading frame.

For each and every polynucleotide of the invention there is also provided a polynucleotide complementary to it.

Vectors, Host Cells, and Expression Systems

The invention also relates to vectors that comprise a polynucleotide or polynucleotides of the invention, host cells that are genetically engineered with vectors of the invention and the production of polypeptides of the invention by recombinant techniques. Cell-free translation systems can also be employed to produce such proteins using RNAs derived from the DNA constructs of the invention

Recombinant STAAU_R2 polypeptides of the present invention may be prepared by processes well known to those skilled in the art from genetically engineered host cells comprising expression systems. Accordingly, in a further aspect, the present invention relates to expression systems that comprise a STAAU_R2 polynucleotide or polynucleotides of the present invention, to host cells which are genetically engineered with such expression systems, and to the production of polypeptides of the invention by recombinant techniques.

For recombinant production of STAAU_R2 polypeptides of the invention, host cells can be genetically engineered to incorporate expression systems or portions thereof or polynucleotides of the invention. Representative examples of appropriate hosts include bacterial cells (Gram positive and Gram negative), fungal cells, insect cells, animal cells and plant cells. Polynucleotides are introduced to bacteria by standard chemical treatment protocols, such as the induction of competence to take up DNA by treatment with calcium chloride (Sambrook et al., supra). Introduction of polynucleotides into fungal (e.g., yeast) host cells is effected, if desired, by standard chemical methods, such as lithium acetate—mediated transformation.

A great variety of expression systems are useful to produce STAAU_R2 polypeptides of the invention. Such vectors include among others, chromosomal-, episomal- and virus-derived vectors. For example, vectors derived from bacterial plasmids, from bacteriophages, from transposons, from yeast episomes, from insertion elements, from yeast chromosomal elements, from viruses, and from vectors derived from combinations thereof, are useful in the invention.

STAAU_R2 polypeptides of the invention are recovered and purified from recombinant cell cultures by well-known methods including ammonium sulfate or ethanol precipitation, acid or urea extraction, anion or cation exchange chromatography, phosphocellulose chromatography, hydrophobic interaction chromatography, affinity chromatography, hydroxylapatite chromatography, and lectin chromatography. Well known techniques for refolding may be employed to regenerate an active conformation when the STAAU_R2 polypeptide is denatured during isolation and/or purification.

Diagnostic, Prognostic, Serotyping, and Mutation Assays

This invention is also related to the use of STAAU_R2 polynucleotides and polypeptides of the invention for use as diagnostic reagents. Detection of S. aureus STAAU_R2 polynucleotides and/or polypeptides in a eukaryote, particularly a mammal, and especially a human, will provide a diagnostic method for diagnosis of disease, staging of disease or response of an infectious organism to drugs. Eukaryotes, particularly mammals, and especially humans, particularly those infected or suspected to be infected with an organism comprising the S. aureus STAAU_R2 gene or protein, may be detected at the nucleic acid or amino acid level by a variety of well known techniques as well as by methods provided herein.

Polypeptides and polynucleotides for prognosis, diagnosis or other analysis may be obtained from a putatively infected and/or infected individual's bodily materials. Polynucleotides from any of these sources, particularly DNA or RNA, may be used directly for detection or may be amplified enzymatically by using PCR or any other amplification technique prior to analysis. RNA, particularly mRNA, cDNA and genomic DNA may also be used in the same ways. Using amplification, characterization of the species and strain of infectious or resident organism present in an individual, may be made by an analysis of the genotype of a selected polynucleotide of the organism. Deletions and insertions can be detected by a change in size of the amplified product in comparison to a genotype of a reference sequence selected from a related organism, preferably a different species of the same genus or a different strain of the same species.

Point mutations can be identified by hybridizing amplified DNA to labeled STAAU_R2 polynucleotide sequences. Perfectly or significantly matched sequences can be distinguished from imperfectly or more significantly mismatched duplexes by DNase or RNase digestion, for DNA or RNA respectively, or by detecting differences in melting temperatures or renaturation kinetics. Polynucleotide sequence differences may also be detected by alterations in the electrophoretic mobility of polynucleotide fragments in gels as compared to a reference sequence. This may be carried out with or without denaturing agents. Polynucleotide differences may also be detected by direct DNA or RNA sequencing. See, for example, Myers et al, (1985) Science 230, 1242. Sequence changes at specific locations also may be revealed by nuclease protection assays, such as RNase, V1 and S1 protection assay or a chemical cleavage method. See, for example, Cotton et al., (1985) Proc. Natl. Acad. Sci., USA 85, 4397-4401.

In another embodiment, an array of oligonucleotide probes comprising STAAU_R2 nucleotide sequence or fragments thereof can be constructed to conduct efficient screening of, for example, genetic mutations, serotype, taxonomic classification or identification. Array technology methods are well known and have general applicability and can be used to address a variety of questions in molecular genetics including gene expression, genetic linkage, and genetic variability (see, for example, Chee et al., (1996) Science 274, 610).

Thus in another aspect, the present invention relates to a diagnostic kit which comprises: (a) a polynucleotide of the present invention, preferably the nucleotide sequence of SEQ ID NO: 1, or a fragment thereof; (b) a nucleotide sequence complementary to that of (a); (c) a polypeptide of the present invention, preferably the polypeptide of SEQ ID NO: 2 or a fragment thereof; or (d) an antibody to a polypeptide of the present invention, preferably to the polypeptide of SEQ ID NO: 2 or fragment thereof.

It will be appreciated that in any such kit, (a), (b), (c) or (d) may comprise a substantial component. Such a kit will be of use in diagnosing a disease or susceptibility to a disease, among others.

This invention also relates to the use of STAAU_R2 polynucleotides of the present invention as diagnostic reagents. Detection of a mutated form of a polynucleotide of the invention, preferably, SEQ ID NO: 1, which is associated with a disease or pathogenicity will provide a diagnostic tool that can add to, or define, a diagnosis of a disease, a prognosis of a course of disease, a determination of a stage of disease, or a susceptibility to a disease, which results from under-expression, over-expression or altered expression of the polynucleotide. Organisms, particularly infectious organisms, carrying mutations in such polynucleotide may be detected at the polynucleotide level by a variety of techniques, such as those described elsewhere herein.

The STAAU_R2 nucleotide sequences of the present invention are also valuable for organism chromosome identification. The sequence is specifically targeted to, and can hybridize with, a particular location on an organism's chromosome, particularly to a S. aureus chromosome. The mapping of relevant sequences to chromosomes according to the present invention may be an important step in correlating those sequences with pathogenic potential and/or an ecological niche of an organism and/or drug resistance of an organism, as well as the essentiality of the gene to the organism. Once a sequence has been mapped to a precise chromosomal location, the physical position of the sequence on the chromosome can be correlated with genetic map data. Such data may be found on-line in a sequence database. The relationship between genes and diseases that have been mapped to the same chromosomal region are then identified through known genetic methods, for example, through linkage analysis (coinheritance of physically adjacent genes) or mating studies, such as by conjugation.

The differences in a polynucleotide and/or polypeptide sequence between organisms possessing a first phenotype and organisms possessing a different, second different phenotype can also be determined. If a mutation is observed in some or all organisms possessing the first phenotype but not in any organisms possessing the second phenotype, then the mutation is likely to be the causative agent of the first phenotype.

Polypeptides and polynucleotides for prognosis, diagnosis or other analysis may be obtained from a putatively infected and/or infected individual's bodily materials. Particularly DNA or polynucleotides, from any of these sources may be used directly for detection or may be amplified enzymatically using PCR or other amplification technique with oligonucleotide amplification primers derived from the polynucleotide sequence of S. aureus STAAU_R2. RNA, particularly mRNA, or RNA reverse transcribed to cDNA, is also useful for diagnostics. Following amplification of a S. aureus STAAU_R2-related polynucleotide from a sample, characterization of the species and strain of infecting or resident organism is made by an analysis of the amplified polynucleotide relative to one or more reference polynucleotides or sequences relative to a standard from a related organism (i.e. a known strain of S. aureus).

The invention further provides a process for diagnosing bacterial infections such as those caused by S. aureus, the process comprising determining from a sample derived from an individual, such as a bodily material, an increased level of expression of a polynucleotide having a sequence disclosed in SEQ ID NO: 1 relative to a sample taken from a non-diseased individual. Increased or decreased expression of a STAAU_R2 polynucleotide can be measured using any one of the methods well known in the art for the quantitation of polynucleotides, such as, for example, PCR, RT-PCR, RNase protection, Northern blotting and other hybridization methods, and spectrometry.

In addition, a diagnostic assay in accordance with the invention for detecting over-expression of STAAU_R2 polypeptide compared to normal control tissue samples may be used to detect the presence of an infection, for example. Assay techniques that can be used to determine levels of a S. aureus DnaN polypeptide, in a sample derived from a host, such as a bodily material, are well-known to those of skill in the art. Such assay methods include radioimmunoassays, competitive-binding assays, Western Blot analysis, antibody sandwich assays, antibody detection and ELISA assays.

Gridding and Polynucleotide Subtraction of S. aureus Genomic Sequences

The STAAU_R2 polynucleotides of the invention may be used as components of polynucleotide arrays, preferably high density arrays or grids. These high density arrays are particularly useful for diagnostic and prognostic purposes. For example, a set of spots each comprising a different gene, and further comprising a polynucleotide or polynucleotides of the invention, may be used for probing, such as hybridization or nucleic acid amplification, using a probe obtained or derived from a bodily sample, to determine the presence a particular polynucleotide sequence or related sequence in an individual.

Antibodies Specific for S. aureus Peptides or Polypeptides

The STAAU_R2 polypeptides and polynucleotides of the invention or variants thereof, or cells expressing them are useful as immunogens to produce antibodies immunospecific for such polypeptides or polynucleotides, respectively.

In certain preferred embodiments of the invention there are provided antibodies against S. aureus STAAU_R2 polypeptides or polynucleotides encoding them. Antibodies against STAAU_R2-polypeptide or STMU R2-polynucleotide are useful for treatment of infections, particularly bacterial infections.

Antibodies generated against the polypeptides or polynucleotides of the invention are obtained by administering the polypeptides and/or polynucleotides of the invention or epitope-bearing fragments of either or both, analogues of either or both, or cells expressing either or both, to an animal, preferably a nonhuman, using routine protocols. For preparation of monoclonal antibodies, any technique known in the art that provides antibodies produced by continuous cell line cultures is useful. Examples include various techniques, such as those in Kohler, G. and Milstein, C., Nature 256: 495497 (1975); Kozbor et al., Immunology Today 4: 72 (1983); and Cole et al., pg. 96-96 in Monoclonal Anbitodies and Cancer Therapy, Alan R. Liss, Inc. (1985).

Techniques for the production of single chain antibodies (U.S. Pat. No. 4,946,968) can be adapted to produce single chain antibodies to polypeptides or polynucleotides of this invention. Also, transgenic mice, or other mammals, are useful to express humanized antibodies immunospecific to the polypeptides or polynucleotides of the invention.

When antibodies are administered therapeutically, the antibody or variant thereof is preferably modified to make it less immunogenic in the individual. For example, if the individual is human the antibody is most preferably “humanized,” where the complementarity determining region or regions of the hybridoma-derived antibody has been transplanted into a human monoclonal antibody, for example as described in Jones et al. (1986), Nature 321, 522-525 or Tempest et al., (1991) Biotechnology 9, 266-273.

Alternatively, phage display technology is useful to select antibody genes with binding activities towards a STAAU_R2 polypeptide of the invention. In one possible scheme, antibody fragments specific for S. aureus STAAU_R2 are selected from an immune library of antibody genes expressed as fusions with coat protein of filamentous phage. Alternatively, naive libraries are screened by phage display techniques to identify genes encoding antibodies specified for STAAU_R2 or from naive libraries [McCafferty, et al., (1990), Nature 348, 552-554; Marks, et al., (1992) Biotechnology 10, 969-783; a recent reference is de Haard et al. (1999) J Biol Chem 274: 18218-18230]. The ability to recover, for various targets, antibodies with subnanomolar affinities obviates the need for immunization. The affinity of these antibodies can also be improved by, for example, chain shuffling [Clackson et al., (1991 j Nature 352: 628].

The above-described antibodies may be employed to isolate or to identify clones expressing the polypeptides or polynucleotides of the invention, for example to purify the polypeptides or polynucleotides by immunoaffinity chromatography.

A variant polypeptide or polynucleotide of the invention, such as an antigenically or immunologically equivalent derivative or a fusion protein of the polypeptide is also useful as an antigen to immunize a mouse or other animal such as a rat or chicken. A fused protein provides stability to the polypeptide acting as a carrier, or acts as an adjuvant or both. Alternatively, the antigen is associated, for example by conjugation, with an immunogenic carrier protein, such as bovine serum albumin, keyhole limpet haemocyanin or tetanus toxoid. Alternatively, when antibodies are to be administered therapeutically, alternatively a multiple antigenic polypeptide comprising multiple copies of the polypeptide, or an antigenically or immunologically equivalent polypeptide thereof may be sufficiently antigenic to improve immunogenicity so as to obviate the use of a carrier.

In accordance with an aspect of the invention, there is provided the use of a STAAU_R2 polynucleotide of the invention for therapeutic or prophylactic purposes, in particular genetic immunization. The use of a STAAU_R2 polynucleotide of the invention in genetic immunization preferably employs a suitable delivery method such as direct injection of plasmid DNA into muscles [Wolff et al., Hum Mol Genet (1992) 1: 363, Manthorpe et al., Hum. Gene Ther. (1983) 4: 419], delivery of DNA complexed with specific protein carriers [Wu et al., J Biol. Chem. (1989).264: 16985], coprecipitation of DNA with calcium phosphate [Benvenisty & Reshef, PNAS USA, (1986) 83: 9551], encapsulation of DNA in various forms of liposomes [Kaneda et al., Science (1989) 243: 375], particle bombardment [Tang et al., Nature (1992) 356:152, Eisenbraun et al., DNA Cell Biol (1993) 12: 791] or in vivo infection using cloned retroviral vectors [Seeger et al., PNAS USA (1984) 81: 5849].

Antagonists and Agonists: Assays and Molecules

The invention is based in part on the discovery that STAAU_R2 is a target for the bacteriophage 44AHJD ORF 25, Twort ORF168, G1 ORF 240 inhibitory factors. Applicants have recognized the utility of the interaction in the development of antibacterial agents. Specifically, the inventors have recognized that 1) STAAU_R2 is a critical target for bacterial inhibition; 2) 44AHJD ORF 25, Twort ORF 168, G1 ORF 240 or derivatives or functional mimetics thereof are useful for inhibiting bacterial growth; and 3) the interaction between STAAU_R2 of S. aureus and 44AHJD ORF 25, Twort ORF 168, G1 ORF 240 may be used as a target for the screening and rational design of drugs or antibacterial agents. In addition to methods of directly inhibiting STAAU_R2 activity, methods of inhibiting STAAU_R2 expression are also attractive for antibacterial activity.

In several embodiments of the invention, there are provided methods for identifying compounds which bind to or otherwise interact with and inhibit or activate an activity or expression of a polypeptide and/or polynucleotide of the invention comprising: contacting a polypeptide and/or polynucleotide of the invention with a compound to be screened under conditions to permit binding to or other interaction between the compound and the polypeptide and/or polynucleotide to assess the binding to or other interaction with the compound, such binding or interaction preferably being associated with a second component capable of providing a detectable signal in response to the binding or interaction of the polypeptide and/or polynucleotide with the compound; and determining whether the compound binds to or otherwise interacts with and activates or inhibits an activity or expression of the polypeptide and/or polynucleotide by detecting the presence or absence of a signal generated from the binding or interaction of the compound with the polypeptide and/or polynucleotide.

Potential antagonists include, among others, small organic molecules, peptides, polypeptides and antibodies that bind to a polynucleotide and/or polypeptide of the invention and thereby inhibit or extinguish its activity or expression. Potential antagonists also may be small organic molecules, a peptide, a polypeptide such as a closely related protein or antibody that binds the same sites on a binding molecule, such as a binding molecule, without inducing STAAU_R2-induced activities, thereby preventing the action or expression of S. aureus STAAU_R2 polypeptides and/or polynucleotides by excluding S. aureus STAAU_R2 polypeptides and/or polynucleotides from binding.

Potential antagonists also include a small molecule that binds to and occupies the binding site of the polypeptide thereby preventing binding to cellular binding molecules, such that normal biological activity is prevented. Cellular binding molecules include but are not limited to proteins involved in DNA replication. Examples of cellular binding molecules include DNA polymerase III (x and 6 subunits, DNA polymerase 1, DNA polymerase 11, DNA polymerase V, DNA ligase and MutS polypeptides.

Examples of small molecules include but are not limited to small organic molecules, peptides or peptide-like molecules. Other potential antagonists include antisense molecules [see Okano, (1991) J. Neurochem. 56, 560; see also Oligodeoxynucleotides As Antisense Inhibitors of Gene Expression, CRC Press, Boca Raton, Fla. (1988), for a description of these molecules]. Preferred potential antagonists include compounds related to and variants of 44AHJD ORF 25, Twort ORF 168, G1 ORF 240 and of STAAU_R2. Other examples of potential polypeptide antagonists include antibodies or, in some cases, oligonucleotides or proteins which are closely related to the ligands, substrates, receptors, enzymes, etc., as the case may be, of the polypeptide, e.g., a fragment of the ligands, substrates, receptors, enzymes, etc.; or small molecules which bind to the polypeptide of the present invention but do not elicit a response, so that the activity of the polypeptide is prevented.

Compounds may be identified from a variety of sources, for example, cells, cell-free preparations, chemical libraries, and natural product mixtures. These substrates and ligands may be natural substrates and ligands or may be structural or functional mimetics. See, e.g., Coligan et al., Current Protocols in Immunology 1(2): Chapter 5 (1991). Peptide modulators can also be selected by screening large random libraries of all possible peptides of a certain length.

Compounds derived from the polypeptide sequence of 44AHJD ORF25, Twort ORF 168 or G1 ORF 240 could represent fragments representing small overlapping peptides spanning the entire amino acid sequence of these ORFs. Fragments of 44AHJD ORF25, Twort ORF 168 or G1 ORF 240 can be produced as described above.

Certain of the polypeptides of the invention are biomimetics, functional mimetics of the natural S. aureus STAAU_R2 polypeptide. These functional mimetics are useful for, among other things, antagonizing the activity of S. aureus STAAU_R2 polypeptide or as an antigen or immunogen in a manner described above. Functional mimetics of the polypeptides of the invention include but are not limited to truncated polypeptides. For example, preferred functional mimetics include a polypeptide comprising the polypeptide sequence set forth in SEQ ID NO: 2 lacking 20, 30, 40, 50, 100, 200, 300, 325 amino- or carboxy-terminal amino acid residues, including fusion proteins comprising one or more of these truncated sequences. Polynucleotides encoding each of these functional mimetics may be used as expression cassettes to express each mimetic polypeptide. It is preferred that these cassettes comprise-5′ and 3′ restriction sites to allow for a convenient means to ligate the cassettes together when desired. It is further preferred that these cassettes comprise gene expression signals known in the art or described elsewhere herein.

Screening Assays According to the Invention

It is desirable to devise screening methods to identify compounds which stimulate or which inhibit the function of the STAAU_R2 polypeptide or polynucleotide of the invention. Accordingly, the present invention provides for a method of screening compounds to identify those that modulate the function of a polypeptide or polynucleotide of the invention. In general, antagonists may be employed for therapeutic and prophylactic purposes. It is contemplated that an agonist of STAAU_R2 may be useful, for example, to enhance the growth rate of bacteria in a sample being cultured for diagnostic or other purposes.

It has been determined that STAAU_R2 is a target for bacteriophage 44AHJD ORF 25, Twort ORF168 and G1 ORF 240 products, which acts as inhibitory factors. Applicants have recognized the utility of the interaction in the development of antibacterial agents. Polypeptide and/or polynucleotide targets such as STAAU_R2 are critical targets for bacterial inhibition. S. aureus bacteriophage 44AHJD ORF 25, Twort ORF168, G1 ORF 240_or derivatives or functional mimetics thereof are useful for inhibiting bacterial growth and the interaction, binding, inhibition and/or activation which occurs between polypeptides, such as for example STAAU_R2 of S. aureus and 44AHJD ORF 25, Twort ORF168, G1 ORF 240 may be used for the screening and rational design of drugs or antibacterial agents. In addition to methods for directly inhibiting a target such as STAAU_R2 activity, methods of inhibiting a target such as STAAU_R2 expression are also attractive for antibacterial activity.

In preferred embodiments, the method involves the interaction of an inhibitory ORF product or fragment thereof with the corresponding bacterial target or fragment thereof that maintains the interaction with the ORF product or fragment. Interference with the interaction between the components can be monitored, and such interference is indicative of compounds that can inhibit, activate, or enhance the activity of the target molecule.

a. Binding Assays

There are a number of methods of examining binding of a candidate compound to a protein target such as STAAU_R2 and a polypeptide comprising amino acid sequence of SEQ ID NO: 2, or fragments thereof. Screening methods that measure the binding of a candidate compound to the STAAU_R2 polypeptide or polynucleotide, or to cells or supports bearing the polypeptide or a fusion protein comprising the polypeptide, by means of a label directly or indirectly associated with the candidate compound, are useful in the invention.

The screening method may involve competition for binding of a labeled competitor such as 44AHJD ORF 25, Twort ORF 168, G1 ORF 240 or a fragment that is competent to bind STAAU_R2.

Non-limiting examples of screening assays [Reviewed in Sittampalam et al. 1997 Curr Opin Chem Biol. 3:384-91] in accordance with the present invention include the following.

i.) Fluorescence Resonance Energy Transfer (FRET)

A method of measuring inhibition of binding of two proteins using fluorescence resonance energy transfer [FRET; de Angelis, 1999, Physiological Genomics]. FRET is a quantum mechanical phenomenon that occurs between a fluorescence donor (D) and a fluorescence acceptor (A) in close proximity (usually <100 A of separation.) if the emission spectrum of D overlaps with the excitation spectrum of A. Variants of the green fluorescent protein (GFP) from the jellyfish Aequorea Victoria are fused to a polypeptide or protein and serve as D-A pairs in a FRET scheme to measure protein-protein interaction. Cyan (CFP: D) and yellow (YFP: A) fluorescence proteins are linked with STAAU_R2 polypeptide, or a fragment of STAAU_R2 and 44AHJD ORF 25, Twort ORF 168, G1 ORF 240 protein respectively. Under optimal proximity, interaction between STAAU_R2, or a fragment of STAAU_R2, and 44AHJD ORF 25, Twort ORF 168, G1 ORF 240 causes a decrease in intensity of CFP fluorescence concomitant with an increase in YFP fluorescence.

The addition of a candidate modulator to the mixture of appropriately labeled STAAU_R2 and 44AHJD ORF 25, Twort ORF 168, G1 ORF 240 protein, will result in an inhibition of energy transfer evidenced by, for example, a decease in YFP fluorescence at a given concentration of 44AHJD ORF 25, Twort ORF 168, G1 ORF 240 relative to a sample without the candidate inhibitor.

ii.) Fluorescence Polarization

Fluorescence polarization measurement is another useful method to quantitate protein-protein binding. The fluorescence polarization value for a fluorescently-tagged molecule depends on the rotational correlation time or tumbling rate. Protein complexes, such as those formed by S. aureus STAAU_R2 polypeptide, or a fragment of STAAU_R2 associating with a fluorescently labeled polypeptide (e.g., 44AHJD ORF 25, Twort ORF 168, G1 ORF 240 or a binding fragment thereof), have higher polarization values than does the fluorescently labeled polypeptide. Inclusion of a candidate inhibitor of the STAAU_R2 interaction results in a decrease in fluorescence polarization relative to a mixture without the candidate inhibitor if the candidate inhibitor disrupts or inhibits the interaction of STAAU_R2 with its polypeptide binding partner. It is preferred that this method be used to characterize small molecules that disrupt the formation of polypeptide or protein complexes.

iii.) Surface Plasmon Resonance

Another powerful assay to screen for inhibitors of a protein: protein interaction is surface plasmon resonance. Surface plasmon resonance is a quantitative method that measures binding between two (or more) molecules by the change in mass near a sensor surface caused by the binding of one protein or other biomolecule from the aqueous phase (analyte) to a second protein or biomolecule immobilized on the sensor (ligand). This change in mass is measured as resonance units versus time after injection or removal of the second protein or biomolecule (analyte) and is measured using a Biacore Biosensor (Biacore AB) or similar device. STAAU_R2, or a polypeptide comprising fragment of STAAU_R2, could be immobilized as a ligand on a sensor chip (for example, research grade CM5 chip; Biacore AB) using a covalent linkage method (e.g. amine coupling in 10 mM sodium acetate [pH 4.5]). A blank surface is prepared by activating and inactivating a sensor chip without protein immobilization. Alternatively, a ligand surface can be prepared by noncovalent capture of ligand on the surface of the sensor chip by means of a peptide affinity tag, an antibody, or biotinylation. The binding of 44AHJD ORF 25, Twort ORF 168 or G1 ORF 240 to STAAU_R2, or a fragment of STAAU_R2, is measured by injecting purified 44AHJD ORF 25, Twort ORF 168 or G1 ORF 240 over the ligand chip surface. Measurements are performed at any desired temperature between 4° C. and 37° C. Conditions used for the assay (i.e., those permitting binding) are as follows: 25 mM HEPES-KOH (pH 7.6), 150 mM sodium chloride, 15% glycerol, 1 mM dithiothreitol, and 0.001% Tween 20 with a flow rate of 10 ul/min. Preincubation of the sensor chip with candidate inhibitors will predictably decrease the interaction between 44AHJD ORF 25, Twort ORF 168 or G1 ORF 240 and STAAU_R2. A decrease in 44AHJD ORF 25, Twort ORF 168 or G1 ORF 240 binding, detected as a reduced response on sensorgrams and measured in resonance units, is indicative of competitive binding by the candidate compound.

iv.) Scintillation Proximity Assay

A scintillation proximity assay (SPA) may be used to characterize the interaction between a S. aureus STAAU_R2 polypeptide, or a fragment of STAAU_R2 polypeptide, for example comprising the amino acid sequence of SEQ ID NO: 2, or a part thereof, and another polypeptide. The SPA relies in a solid-phase substrate, such as beads or the plastic of a microtitre plate, into which a scintillant has been incorporated. For the assay, the target protein, for example a S. aureus STAAU_R2 polypeptide, is coupled to the beads or to the surface of the plate, either covalently through activated surface chemistries or non-covalently through a peptide affinity tag, an antibody, or biotinylation. Addition of a radiolabeled binding polypeptide, for example [³²P]-radiolabeled 44AHJD ORF 25, Twort ORF 168 or G1 ORF 240, results in close proximity of the radioactive source molecule to the scintillant. As a consequence, the radioactive decay excites the scintillant contained within the bead or within the plastic of the plate and detectable light is emitted. Compounds that prevent the association between immobilized S. aureus STAAU_R2 polypeptide and radiolabeled 44AHJD ORF 25, Twort ORF 168 or G1 ORF 240 will diminish the scintillation signal. The SPA thus represents an example of an ideal technology with which to screen for inhibitors of the STAAU_R244AHJD ORF 25, STAAU_R2-Twort ORF 168 or STAAU_R2-G1 ORF 240 interactions because it is readily adapted to high-throughput, automated format and because of its sensitivity for detection of protein-protein interactions with K_{D} values in the micromolar to nanomolar ranges.

v.) Bio Sensor Assay

ICS biosensors have been described by AMBRI (Australian Membrane Biotechnology Research Institute; http//www.ambri.com.au/). In this technology, the self-association of macromolecules such as STAAU_R2, or a fragment of STAAU_R2, and bacteriophage 44AHJD ORF 25, Twort ORF 168 or G1 ORF 240, is coupled to the closing of gramacidin-facilitated ion channels in suspended membrane bilayers and hence to a measurable change in the admittance (similar to impedence) of the biosensor. This approach is linear over six order of magnitude of admittance change and is ideally suited for large scale, high through-put screening of small molecule combinatorial libraries.

vi.) Phage Display

Phage display is a powerful assay to measure protein:protein interaction. In this scheme, proteins or peptides are expressed as fusions with coat proteins or tail proteins of filamentous bacteriophage. A comprehensive monograph on this subject is Phage Display of Peptides and Proteins. A Laboratory Manual edited by Kay et al. (1996) Academic Press. For phages in the Ff family that include M13 and fd, gene III protein and gene VIII protein are the most commonly-used partners for fusion with foreign protein or peptides. Phagemids are vectors containing origins of replication both for plasmids and for bacteriophage. Phagemids encoding fusions to the gene III or gene VIII can be rescued from their bacterial hosts with helper phage, resulting in the display of the foreign sequences on the coat or at the tip of the recombinant phage.

In the simplest assay, purified recombinant STAAU_R2 protein, or a fragment of STAAU_R2, could be immobilized in the wells of a microtitre plate and incubated with phages displaying 44AHJD ORF 25, Twort ORF 168 or G1 ORF 240 in fusion with the gene III protein. Washing steps are performed to remove unbound phages and bound phages are detected with monoclonal antibodies directed against phage coat protein (gene VIII protein). An enzyme-linked secondary antibody allows quantitative detection of bound fusion protein by fluorescence, chemiluminescence, or colourimetric conversion. Screening for inhibitors is performed by the incubation of the compound with the immobilized target before the addition of phages. The presence of an inhibitor will specifically reduce the signal in a dose-dependent manner relative to controls without inhibitor.

It is important to note that in assays of protein-protein interaction, it is possible that a modulator of the interaction need not necessarily interact directly with the domain(s) of the proteins that physically interact. It is also possible that a modulator will interact at a location removed from the site of protein-protein interaction and cause, for example, a conformational change in the STAAU_R2 polypeptide. Modulators (inhibitors or agonists) that act in this manner can be termed allosteric effectors and are of interest since the change they induce may modify the activity of the STAAU_R2 polypeptide.

b. Assays of STAAU_R2 Functional Activity

Non-limiting examples of assays to assess the functional enzymatic activity of STAAU_R2, or fragments thereof, variant or homolog thereof, include the measurement of stimulation of in vitro DNA replication. There are number of methods of measuring the DNA synthesis stimulation of a polypeptide comprising the amino acid sequence of STAAU_R2.

For example, an assay for STAAU_R2 activity could involved the measurement of radiolabeled nucleotide incorporated into cellular DNA. Samples (0.5 ml) are withdrawn from cultures at appropriate time intervals and mixed with 4.5 μl of labeling solution (0.2 μCi/ml of ³H-thymidine (73 Ci/mmol, NEN Life Science Products, Inc.) and 70 pmol of unlabeled thymidine). After 15 minutes of reaction, incorporation is stopped by adding 5 μl of 0.2% NaN₃ and 5 μl of 30 μg/ml unlabeled thymidine. Samples are precipitated with 10% (w/v) trichloroacetic acid and filtered through glass fiber filters (GF-C, Whatman). The results are expressed as ³H-thymidine counts incorporated, normalized to the OD of the culture.

DNA synthesis could be measured by using a soluble cell-free in vitro system based on the use of a variety of different synthetic DNA substrates. The replication assay could involved crude or partially purified cellular proteins extracts. The replication assays could be reconstituted with partially pure or pure form of native proteins or recombinantly produced proteins.

In one cell-free in vitro assay, an extract prepared from S. aureus is supplied to a plasmid substrate, for example a primed circular M13ssDNA substrate, in a reaction including exogenous radiolabeled deoxynucleotide triphosphates (dATP, dTTP, dGTP and dCTP), MgCl₂ and ATP. The reaction is stopped and the products precipitated with trichloroacetic acid, and then filtered. Scintillation counting of the dried filter gives the level of de novo replication.

Another example to assay for STAAU_R2 activity is to measure the level of radiolabeled nucleotide incorporated into DNA in a reconstituted in vitro assay using primed circular ssDNA substrate [Bruck and O'Donnell 2000, J. Biol. Chem. 275: 28971-28983]. The replication reactions typically, contained Tris-HCl [pH 7.5], MgCl₂, BSA, DTT, ATP, dCTP, dGTP, and dATP, [α-³²P]dTTP, EDTA, glycerol, circular primed M13ssDNA, S. aureus SSB, PolC, τ, δ and δ′ and increasing amount of STAAU_R2 polypeptide. Reactions were incubated at 37° C. for 5 min and quenched upon addition of SDS and EDTA. Half of the quenched reaction is analyzed for total DNA synthesis using a DE81 filter paper. The other half is analyzed by electrophoresis on agarose gel. Gels are dried, and radioactive products were visualized following exposure to a phosphorImager screen for the evaluation of the rate of DNA synthesis and processivity.

Testing for inhibitors, for example 44AHJD ORF 25 and Twort ORF168, is performed by the incubation of the compound with the reaction mixtures. The presence of an inhibitor will specifically reduce the signal in a dose-dependent manner relative to controls without inhibitor. Compounds selected for their ability to inhibit interactions between STAAU_R2-44AHJD ORF 25 or STAAU_R2-Twort ORF can be further tested in functional activity assays.

Alternatively, a rapid fluorometric assay that measures the activity of replication enzymes could be developed to measure STAAU_R2 activity. The fluorometric assay is based on the preferential binding of a fluorescent dye to double stranded DNA, for example, de novo synthesized DNA, vs. single stranded DNA and it has been described (Seville et al., 1996. Biotechniques 21:664-672). A reconstituted in vitro assay similar to that described using primed circular ssDNA substrate [Bruck and O'Donnell 2000, J. Biol. Chem. 275:28971-28983] could be developed. The replication reactions would contain Tris-HCl [pH 7.5], MgCl₂, BSA, DTT, ATP, dNTPs, EDTA, glycerol, circular primed M13ssDNA, S. aureus SSB, PolC, τ, δ and δ′ and increasing amount of STAAU_R2 polypeptide. Reactions are incubated at 37° C. for variable times then quenched. The quenched reaction is analyzed for total DNA synthesis by adding PicoGreen™ dye (Molecular Probes, Eugene, Oreg.), incubating 5 min at room temperature, and reading the intensity of fluorescence of PicoGreen (λ_{EX}, 485 nm; λ_{EM}, 525 nm). The sensitivity of the dye and the homogeneous nature of the PicoGreen assay should allow rapid screening in a non-radiometric assay format.

Testing for inhibitors, for example of 44AHJD ORF 25, Twort ORF168 or G1 ORF 240 (of fragments or variants thereof), is performed by the incubation of the compound with the reaction mixtures. The presence of an inhibitor will specifically reduce the fluorescence signal in a dose-dependent manner relative to controls without inhibitor. Compounds selected for their ability to inhibit interactions between STAAU_R2-44AHJD ORF 25, STAAU_R2-Twort ORF 168 or STAAU_R2-G1 ORF 240 can be further tested in functional activity assays.

c. Bacterial Growth Inhibition

Compounds selected for their ability to inhibit interactions between STAAU_R2 and 44AHJD ORF 25, Twort ORF 168 or G1 ORF 240 or to inhibit the STAAU_R2 activity can be further tested in functional assays of bacterial growth. Cultures of S. aureus are grown in the presence of varying concentrations of a candidate compound added directly to the medium or using a vehicle which is appropriate for the delivery of the compound into the cell. For compounds that correspond to polypeptides, the nucleotide sequence encoding said polypeptides can be cloned into a S. aureus expression vector containing an inducible promotor. The expression of the polypeptide could be induced following transfection of cells. For example, the polypeptide may include, but is not limited to the different 44AHJD ORF 25, Twort ORF 168 or G1 ORF 240-derived fragments (e.g. with SEQ ID NO: 8).

Following the induction of expression or the addition of compound, the cultures are then incubated for an additional 4 h at 37° C. During that period of time, the effect of inhibitors on bacterial cell growth may be monitored at 40 min intervals, by measuring, for example, the OD₅₆₅ and the number of colony forming units (CFU) in the cultures. The number of CFU is evaluated as follows: cultures are serially diluted and aliquots from the different cultures are plated out on agar plates. Following incubation overnight at 37° C., the number of colonies is counted. Non-treated cultures of S. aureus are included as negative control.

In another aspect, the present invention relates to a screening kit for identifying agonists, antagonists, ligands, receptors, substrates, enzymes, etc. for a polypeptide and/or polynucleotide of the present invention; or compounds which decrease or enhance the production of such polypeptides and/or polynucleotides, which comprises: (a) a polypeptide and/or a polynucleotide of the present invention; (b) a recombinant cell expressing a polypeptide and/or polynucleotide of the present invention; (c) a cell membrane associated with a polypeptide and/or polynucleotide of the present invention; or (d) an antibody to a polypeptide and/or polynucleotide of the present invention; which polypeptide is preferably that of SEQ ID NO: 2, and for which the polynucleotide is preferably that of SEQ ID NO: 1.

It will be appreciated that in any such kit, (a), (b), (c) or (d) may comprise a substantial component.

It will be readily appreciated by the skilled artisan that a polypeptide and/or polynucleotide of the present invention may also be used in a method for the structure-based design of an agonist, antagonist or inhibitor of the polypeptide and/or polynucleotide, by: (a) determining in the first instance the three-dimensional structure of the polypeptide and/or polynucleotide, or complexes thereof; (b) deducing the three-dimensional structure for the likely reactive site(s), binding site(s) or motif(s) of an agonist, antagonist or inhibitor; (c) synthesizing candidate compounds that are predicted to bind to or react with the deduced binding site(s), reactive site(s), and/or motif(s); and (d) testing whether the candidate compounds are indeed agonists, antagonists or inhibitors. It will be further appreciated that this will normally be an iterative process, and this iterative process may be performed using automated and computer-controlled steps.

Each of the polynucleotide sequences provided herein may be used in the discovery and development of antibacterial compounds. The encoded protein, upon expression, can be used as a target for the screening of antibacterial drugs. Additionally, the polynucleotide sequences encoding the amino terminal regions of the encoded protein or Shine-Dalgarno or other sequence that facilitate translation of the respective mRNA can be used to construct antisense sequences to control the expression of the coding sequence of interest.

The invention also provides the use of the polypeptide, polynucleotide, agonist or antagonist of the invention to interfere with the initial physical interaction between a pathogen or pathogens and a eukaryotic, preferably mammalian, host that is responsible for sequelae of infection. In particular, the molecules of the invention may be used: in the prevention of adhesion of bacteria, in particular Gram positive and/or Gram negative bacteria, to eukaryotic, preferably mammalian, extracellular matrix proteins on in-dwelling devices or to extracellular matrix proteins in wounds; to block bacterial adhesion between eukaryotic, preferably mammalian, extracellular matrix proteins and bacterial STAAU_R2 proteins that mediate tissue damage and/or; to block the normal progression of pathogenesis in infections initiated other than by the implantation of in-dwelling devices or by other surgical techniques.

In accordance with yet another aspect of the invention, there are provided STAAU_R2 antagonists, preferably bacteriostatic or bacteriocidal antagonists.

The antagonists of the invention may be employed, for instance, to prevent, inhibit and/or treat diseases.

Compositions, Kits and Administration

In a further aspect of the invention there are provided compositions comprising a STAAU_R2 polynucleotide and/or a S. aureus STAAU_R2 polypeptide for administration to a cell or to a multicellular organism.

The present invention provides for pharmaceutical compositions comprising a therapeutically effective amount of a polypeptide and/or polynucleotide, such as the soluble form of a polypeptide and/or polynucleotide of the present invention, antagonist peptide or small molecule compound, in combination with a pharmaceutically acceptable carrier or excipient. Such carriers include, but are not limited to, saline, buffered saline, dextrose, water, glycerol, ethanol, and combinations thereof. The pharmaceutical compositions may be administered in any effective, convenient manner including, for instance, administration by topical, oral, anal, vaginal, intravenous, intraperitoneal, intramuscular, subcutaneous, intranasal or intradermal routes among others.

In therapy or as a prophylactic, the active agent may be administered to an individual as an injectable composition, for example as a sterile aqueous dispersion, preferably isotonic.

Alternatively the composition may be formulated for topical application for example in the form of ointments, creams, lotions, eye ointments, eye drops, ear drops, mouthwash, impregnated dressings and sutures and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation. Alternative means for systemic administration include transmucosal and transdermal administration using penetrants such as bile salts or fusidic acids or other detergents. In addition, if a polypeptide or other compounds of the present invention can be formulated in an enteric or an encapsulated formulation, oral administration may also be possible. Administration of these compounds may also be topical and/or localized, in the form of salves, pastes, gels, and the like.

For administration to mammals, and particularly humans, it is expected that the daily dosage level of the active agent will be from 0.01 mg/kg to 10 mg/kg, typically around 1 mg/kg. The physician in any event will determine the actual dosage that will be most suitable for an individual and will vary with the age, weight and response of the particular individual. The above dosages are exemplary of the average case. There can, of course, be individual instances where higher or lower dosage ranges are merited, and such are within the scope of this invention.

As used herein, the term “in-dwelling device” refers to surgical implants, prosthetic devices and catheters, i.e., devices that are introduced to the body of an individual and remain in position for an extended time. Such devices include, but are not limited to, artificial joints, heart valves, pacemakers, vascular grafts, vascular catheters, cerebrospinal fluid shunts, urinary catheters, continuous ambulatory peritoneal dialysis (CAPD) catheters.

The composition of the invention may be administered by injection to achieve a systemic effect against relevant bacteria shortly before insertion of an in-dwelling device. Treatment may be continued after surgery during the in-body time of the device. In addition, the composition could also be used to broaden perioperative cover for any surgical technique to prevent bacterial wound infections, especially S. aureus wound infections.

Many orthopedic surgeons consider that humans with prosthetic joints should be considered for antibiotic prophylaxis before dental treatment that could produce a bacteremia. Deep infection is a serious complication sometimes leading to loss of the prosthetic joint and is accompanied by significant morbidity and mortality. It may therefore be possible to extend the use of the active agent as a replacement for prophylactic antibiotics in this situation.

In addition to the therapy described above, the compositions of this invention may be used generally as a wound treatment agent to prevent adhesion of bacteria to matrix proteins exposed in wound tissue and for prophylactic use in dental treatment as an alternative to, or in conjunction with, antibiotic prophylaxis.

Alternatively, the composition of the invention may be used to bathe an indwelling device immediately before insertion. The active agent will preferably be present at a concentration of 1 mg/ml to 10 mg/ml for bathing of wounds or indwelling devices.

A vaccine composition is conveniently in injectable form. Conventional adjuvants may be employed to enhance the immune response. A suitable unit dose for vaccination is 0.5-5 microgram of antigen/kg, and such dose is preferably administered 1-3 times and with an interval of 1-3 weeks. With the indicated dose range, no adverse toxicological effects will be observed with the compounds of the invention that would preclude their administration to suitable individuals.

Sequence Databases, Sequences in a Tangible Medium, and Algorithms

Polynucleotide and polypeptide sequences form a valuable information resource with which to determine their 2- and 3-dimensional structures as well as to identify further sequences of homology. These approaches are most easily facilitated by storing the sequence in a computer readable medium and then using the stored data in a known macromolecular structure program or to search a sequence database using well-known searching tools, such as GCC.

The polynucleotide and polypeptide sequences of the invention are particularly useful as components in databases useful for search analyses as well as in sequence analysis algorithms. As used in this section entitled “Sequence Databases, Sequences in a Tangible Medium, and Algorithms,” and in claims related to this section, the terms “polynucleotide of the invention” and “polynucleotide sequence of the invention” mean any detectable chemical or physical characteristic of a polynucleotide of the invention that is or may be reduced to or stored in a tangible medium, preferably a computer readable form. For example, chromatographic scan data or peak data, photographic data or scan data therefrom, called bases, and mass spectrographic data. As used in this section entitled Databases and Algorithms and in claims related thereto, the terms “polypeptide of the invention” and “polypeptide sequence of the invention” mean any detectable chemical or physical characteristic of a polypeptide of the invention that is or may be reduced to or stored in a tangible medium, preferably a computer readable form. For example, chromatographic scan data or peak data, photographic data or scan data therefrom, and mass spectrographic data.

The invention provides a computer readable medium having stored thereon polypeptide sequences of the invention and/or polynucleotide sequences of the invention. The computer readable medium can be any composition of matter used to store information or data, including, for example, commercially available floppy disks, tapes, chips, hard drives, compact disks, and video disks.

In a preferred embodiment of the invention there is provided a computer readable medium having stored thereon a member selected from the group consisting of: a polynucleotide comprising the sequence of SEQ ID NO: 1; a polypeptide comprising the sequence of SEQ ID NO: 2; a set of polynucleotide sequences wherein at least one of said sequences comprises the sequence of SEQ ID NO: 1: a set of polypeptide sequences wherein at least one of said sequences comprises the sequence of SEQ ID NO: 2; a data set representing a polynucleotide sequence comprising the sequence of SEQ ID NO: 1; a data set representing a polynucleotide sequence encoding a polypeptide sequence comprising the sequence of SEQ ID NO: 2; a polynucleotide comprising the sequence of SEQ ID NO: 1; a polypeptide comprising the sequence of SEQ ID NO: 2; a set of polynucleotide sequences wherein at least one of said sequences comprises the sequence of SEQ ID NO: 1; a set of polypeptide sequences wherein at least one of said sequences comprises the sequence of SEQ ID NO: 2; a data set representing a polynucleotide sequence comprising the sequence of SEQ ID NO: 1; a data set representing a polynucleotide sequence encoding a polypeptide sequence comprising the sequence of SEQ ID NO: 2.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

The present invention is illustrated in further detail by the following non-limiting examples.

### EXAMPLE 1

Cloning of Inhibitory ORFs from Bacteriophage Genomes

We have used the methodology of a previous invention (PCT International Application WO1999/IB99/02040, filed Dec. 3, 1999) to identify inhibitory ORFs from bacteriophage 44AHJD and Twort. The Staphylococcus aureus propagating strain (PS 44A) (Felix d'Herelle Reference Centre #HER 1101, Ottawa, Canada) was used as a host to propagate its respective phage 44AHJD (Felix d'Herelle Reference Centre #HER101). The Staphylococcus propagating strain (PS Twort) obtained from the Felix d'Herelle Reference Centre maintained by Dr H.-W. Ackermann (Quebec, Canada) (#HER 1048) was used as a host to propagate the phage Twort, also obtained from the Felix d'Herelle Reference Centre (#HER 48).

The Staphylococcus aureus propagating strain PS15 (ATCC 27712), obtained from American Type Culture Collection (Manassas, Va., USA) was used as a host to propagate phage G1. Phage G1 was isolated from a cocktail of S. aureus phages (Bacteriophagum staphylococcum liquidum, lot number 361098) manufactured by BioPharm, Tblisi, Republic of Georgia, by infection of PS15. The cocktail of Staphylococcus aureus phages was purchased from a drugstore in Tblisi.

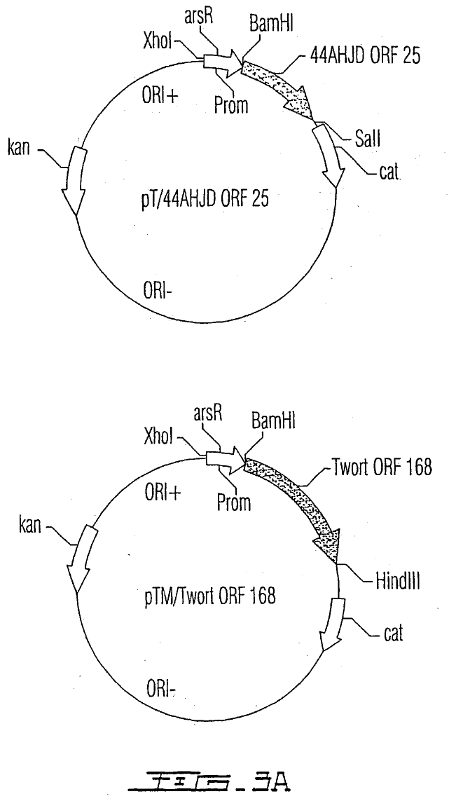

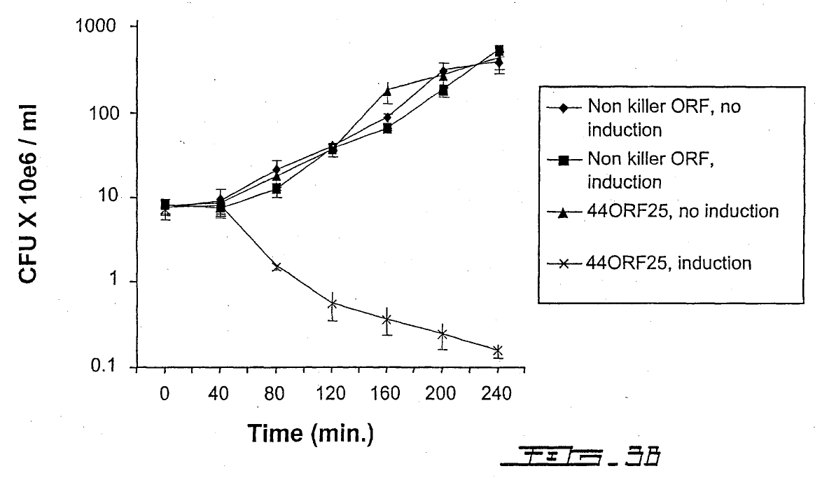

The inhibitory ORF corresponding to SEQ ID NO: 4, 6 and 10 were amplified by polymerase chain reaction (PCR) from genomic DNA derived from phage 44AHJD, Twort and G1 respectively. As examplified in FIG. 3A for 44AHJD ORF 25 and Twort ORF 168, the PCR products were cloned into pT and pTM, two modified versions of the pT0021, a sodium arsenite inducible expression vector [Tauriainen et al., 1997, Appl. Environ. Microbiol. 63:4456-4461]. As shown in FIG. 3B, the density of the culture, as assessed by colony forming units (CFU), for S. aureus clones harboring inhibitory ORFs increased over time under non-induced conditions. Similar growth rates were also observed with transformants harboring a non-inhibitory ORF (labeled as ‘non killer’ on the graphs) under both induced and non-induced conditions. Each graph represents the average obtained from three independent transformants of S. aureus. The expression of 44AHJD ORF 25 and Twort ORF168 inhibit the bacterial growth as observed by the reduction in CFU with time for induced cultures (5.0 uM sodium arsenite). The expression of G1 ORF 240 similarly inhibited the growth of S. aureus (results not shown).

### EXAMPLE 2

### Identification of a S. Aureus Protein Targeted by Bacteriophage 44AHJD ORF 25

A. Generation of GST/ORF 25 Recombinant Protein

Bacteriophage 44AHJD ORF 25 was sub-cloned into pGEX 4T-1 (Pharmacia), an expression vector containing the GST moiety. ORF 25 was obtained by digestion of pT/44AHJD ORF 25 with BamHI and SalI. The DNA fragment containing ORF 25 was gel purified by QiaQuick™ spin columns (Qiagen) and ligated into pGEX 4T-1 (which had been previously digested with BamHI and SalI) to generate pGEX 4T GST/ORF 25. Recombinant expression vectors were identified by restriction enzyme analysis of plasmid minipreps. Large-scale DNA preparations were performed and the resulting insert was sequenced. Test expressions in E. coli BL21 (DE3) Gold cells containing the expression plasmids were performed to identify optimal protein expression conditions. E. coli cells containing the expression constructs were grown in Luria-Bertani Broth at 25° C. to an OD₆₀₀ of 0.4 to 0.6 and induced with 1 mM IPTG for the optimal times and at the optimal temperatures (typically a 2 liter culture of BL21 (DE3) Gold (pGEX 4T/ORF25) grown at 25° C. for 3 hrs).

B. Fusion Protein Purification.

Cells containing GST/ORF 25 fusion protein were suspended in 15 ml lysis buffer/liter of cell culture with GST lysis buffer (20 mM Hepes pH 7.2, 500 mM NaCl, 10% glycerol, 1 mM DTT, 1 mM EDTA, 1 mM benzamidine, and 1 PMSF) and lysed using a French pressure cell followed by three bursts of twenty seconds with an ultra-sonicator at 4° C. Triton X-100 was added to the lysate to a final concentration of 0.1% and mixed for 30 minutes at 4° C. The lysate was centrifuged at 4° C. for 30 minutes at 10,000 rpm in a Sorval SS34 rotor. The supernatant was applied to a 4 ml glutathione sepharose column pre-equilibrated with lysis buffer and allowed to flow by gravity. The column was washed with 10 column volumes of lysis buffer and eluted in 1.5 ml fractions with GST elution buffer (20 mM Hepes pH 8.0, 500 mM NaCl, 10% glycerol, 1 mM DTT, 0.1 mM EDTA, and 25 mM reduced glutathione). The fractions were analyzed by SDS-12.5% PAGE (Laemmli) and proteins were visualized by staining with Coomassie Brilliant Blue R250 stain to assess the amount of eluted GST/ORF 25 protein.

C. Affinity Column Preparation.

GST and GST/ORF25 were dialyzed overnight against affinity chromatography buffer (ACB; 20 mM Hepes pH 7.5, 10% glycerol, 1 mM DTT, and 1 mM EDTA) containing 1 M NaCl. Protein concentrations were determined by Bio-Rad Protein Assay and crosslinked to Affigel 10 resin (Bio-Rad) at protein/resin concentrations of 0, 0.1, 0.5, 1.0, and 2.0 mg/ml. The crosslinked resin was sequentially incubated in the presence of ethanolamine, and bovine serum albumin (BSA) prior to column packing and equilibration with ACB containing 100 mM NaCl.

D. S. aureus Extract Preparation.

Two extracts were prepared from S. aureus cell pellets. One lysate was prepared by French pressure cell lysis followed by sonication, and the other by lysostaphin-mediated digestion followed by sonication. The French pressure cell lysate was prepared by suspending 3 g of frozen S. aureus cells in ABC containing 500 mM NaCl, 1 mM PMSF, and 1 mM benzamidine. The suspended cells were subjected to three passes through the French pressure cell followed by 3 sonication bursts of 20 seconds each, made up to 0.1% Triton X-100, stirred for 30 minutes, and centrifuged at 50,000 rpm for 3 hrs in a Ti70 fixed angle Beckman rotor. The efficiency of cell lysis was low and the resulting lysate (7 ml) contained 2.4 mg/ml protein. The pellet after French pressure cell lysis was subjected to cryogenic grinding in liquid nitrogen in the same buffer with a mortar and pestle. The lysate was made up to 0.1% Triton X-100, stirred for 30 minutes, and centrifuged at 50,000 rpm for 3 hrs in a Ti70 fixed angle Beckman rotor yielding a lysate (10 ml) containing 2.0 mg/ml protein. The cell lysates were pooled, concentrated to 8 ml, and dialyzed overnight in a 3000 Mr cut-off dialysis membrane against ACB containing 1 mM PMSF, 1 mM benzamidine, and 75 mM NaCl. The dialyzed protein extract was removed from the dialysis tubing, centrifuged at 10 000 rpm in a Sorval SS34 rotor for 1 hr, and assayed for protein content (Bio-Rad Protein Assay) and salt concentration (conductivity meter).

E. Affinity Chromatography.

Affinity chromatography was performed using GST and GST ORF25 as ligands coupled to Affigel 10 at protein/resin concentrations of 0, 0.1, 0.5, 1.0, and 2.0 mg/ml. The S. aureus extract was centrifuged at 4° C. in a micro-centrifuge for 15 minutes and 200 ul was applied to 20 ul columns containing 0, 0.1, 0.5, 1.0, and 2.0 mg/ml ligand. ACB containing 100 mM NaCl (200 ul) was applied to a control column containing 2.0 mg/ml ligand. The columns were washed with 10 column volumes ACB containing 100 mM NaCl and sequentially eluted with ACB containing 1% Triton X-100 and 100 mM NaCl (800 ul), ACB containing 1 M NaCl (800 ul), and 1% SDS (160 ul). 40 ul of each eluate was resolved by SDS-12.5% PAGE (Laemmli) and the protein was visualized by silver stain.

F. Identification of S. Aureus DnaN as an 44AHJD ORF 25 Interacting Protein

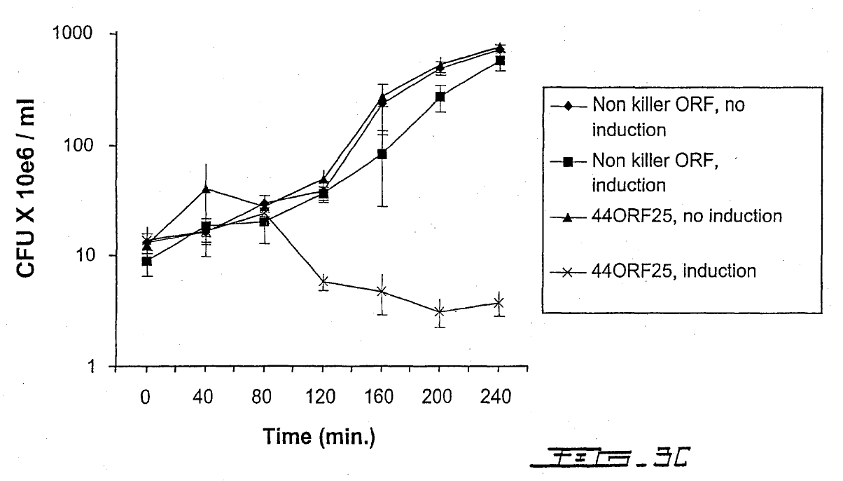

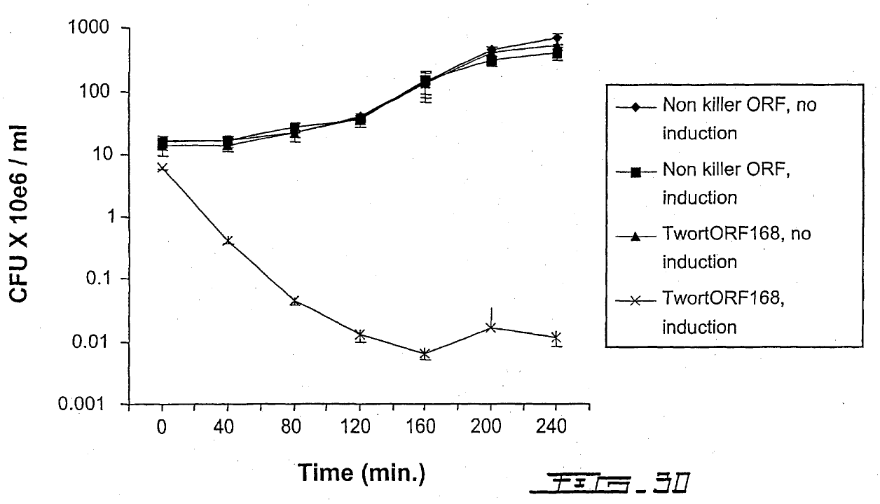

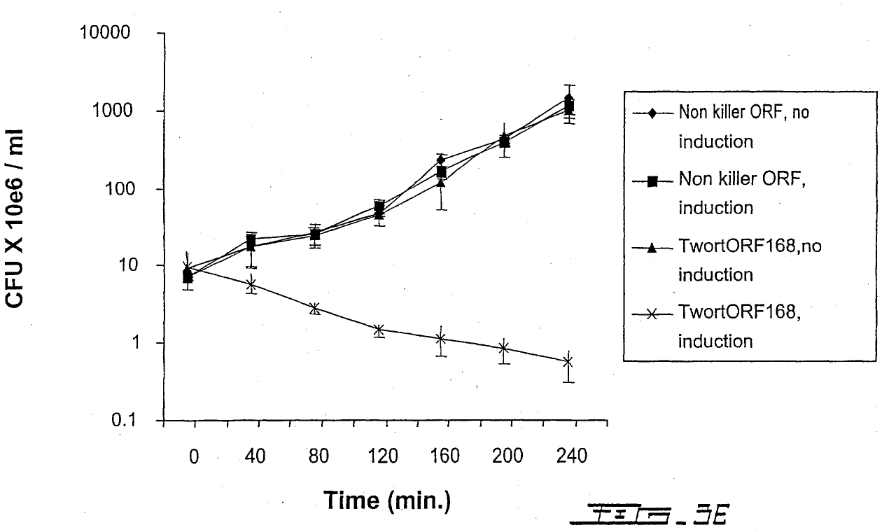

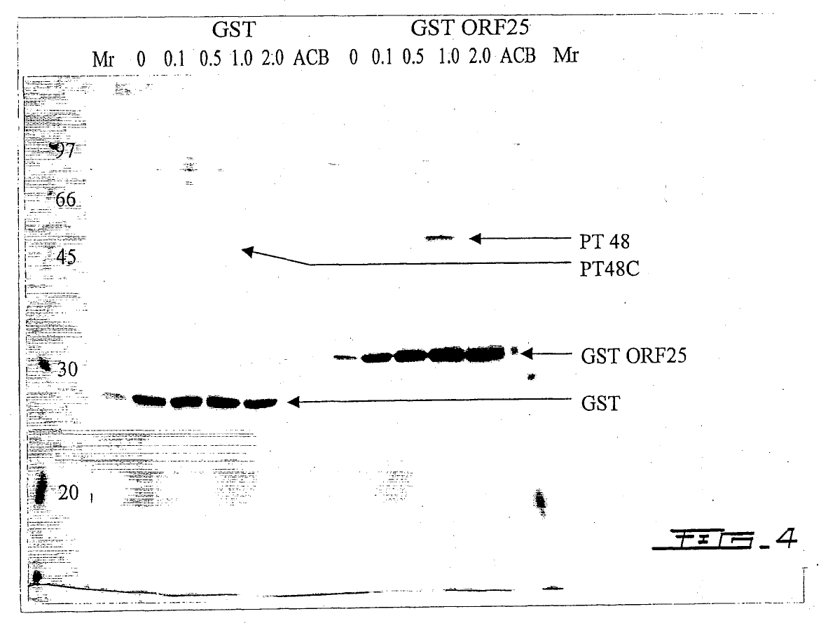

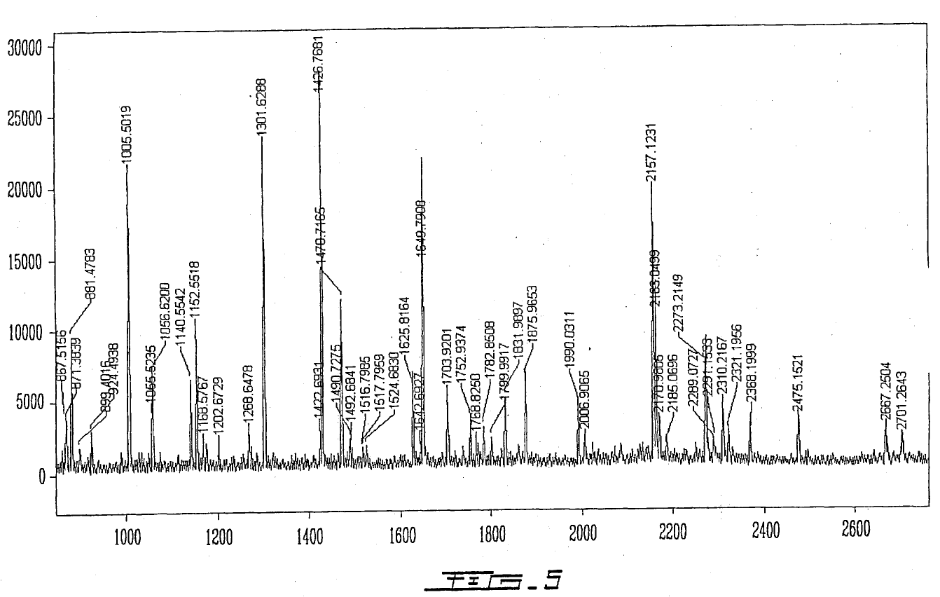

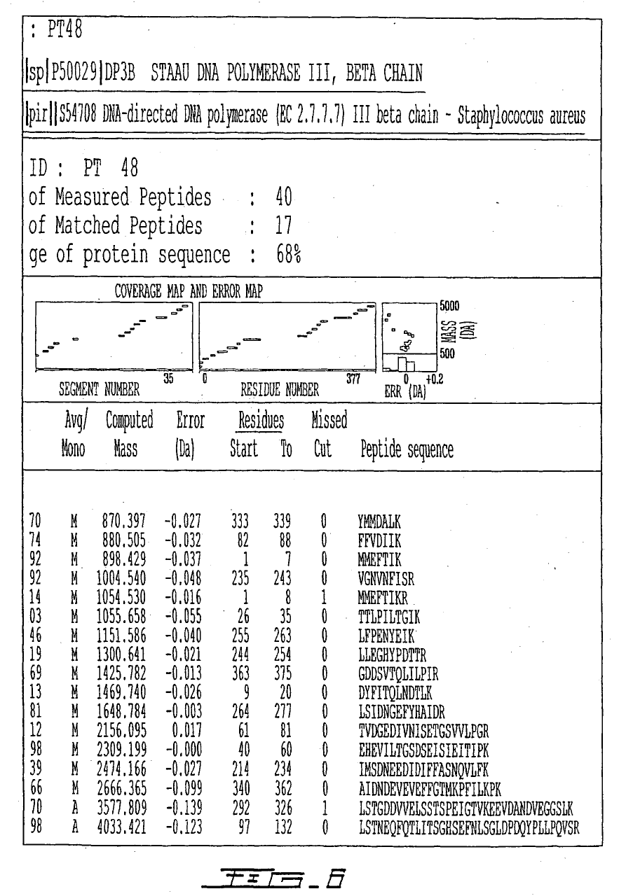

Two S. aureus extracts were used for affinity chromatography with each of the ligands. Two extracts used for affinity chromatography, prepared separately, contained 4.0 and 9.0 mg/ml protein. One candidate interacting protein of 48 kDa (PT48) was observed in the 1% SDS eluates in the initial chromatography experiment (FIG. 4). The candidate protein, PT48 was excised from the SDS-PAGE gels and prepared for tryptic peptide mass determination by MALDI-ToF mass spectrometry [Qin, J., Fenyo, D., Zhao, Y., Hall, W. W., Chao, D. M., Wilson, C. J., Young, R. A. and Chait, B. T. (1997) Anal. Chem. 69, 3995-4001]. High quality mass spectra were obtained (FIG. 5). The PT48 proteins observed in two affinity chromatography experiments were identical as determined by the masses of the tryptic peptides. Computational analysis (http://prowl.rockfeller.edu/cgi-bin/ProFound) of the mass spectrum obtained identifies the corresponding ORF in the S. aureus nucleotide sequence in the University of Oklahoma S. aureus genomic database (http://www.genome.ou.edu/staph.html). The identity of that protein which binds specifically to GST ORF25 is the DNA-directed DNA polymerase III beta subunit (Genbank accession #1084187) (FIG. 6).

The identification of S. aureus STAAU_R2 as an interacting partner of bacteriophage 44ADJH ORF 25 was also validated by surface plasmon resonance (Biacore 2000 Biosensor) using purified recombinant polypeptides. Glutathione-S-transferase (GST)-tagged STAAU_R2 was captured as ligand by an anti-GST antibody which had been covalently coupled to the surface of a CM5 sensor chip; a blank surface with anti-GST antibody and without captured ligand was used as a negative control. Injection of purified 44ADJH ORF 25 protein over the two surfaces indicated specific capture of 44ADJH ORF 25 by immobilized STAAU_R2.

### EXAMPLE 3

### Identification of a S. Aureus Protein Targeted by Bacteriophage Twort ORF168

A. Generation of GST/Twort ORF168 Recombinant Protein

Bacteriophage Twort ORF 168 was sub-cloned into pGEX 4T-1. Plasmid (pTM) containing Twort ORF 168 was purified on a Qiagen column and digested with HindIII, treated with Klenow fragment of E coli DNA polymerase, and digested with BamHI. The DNA restriction products containing the ORF was gel purified by QiAquick spin column (Qiagen) and ligated into pGEX 4T-1 expression vector (prepared by digestion with SalI, treatment with Klenow fragment of E. coli DNA polymerase, and digestion with BamH1, followed by gel purification by QiAquick spin columns). Recombinant expression vectors were identified by restriction enzyme analysis of plasmid minipreps, and large-scale DNA preparations were performed with Qiagen DNA purification columns. Test expressions in E. coli cells containing the expression plasmids were performed to identify optimal protein expression conditions (expressed in BL21(DE3) Gold cells). E. coli cells containing the expression constructs were grown in Luria-Bertani Broth at 37° C. to an OD₆₀₀ of 0.4 to 0.6 and induced with 1 mM IPTG for the optimal times and at the optimal temperatures (2 liters of GST ORF168 at 15° C. for 16 hrs).

B. Fusion Protein Purification.

Cells containing GST ORF 168 fusion protein were suspended in 10 ml lysis buffer/liter of cell culture with GST lysis buffer and lysed by three bursts of twenty seconds with an ultra-sonicator at 4° C. The lysate was centrifuged at 4° C. for 30 minutes at 10 000 rpm in a Beckman JA25.50 rotor. The supernatant was applied to a 4 ml glutathione sepharose column pre-equilibrated with lysis buffer and allowed to flow by gravity. The column was washed with 10 column volumes of lysis buffer and eluted in 4 ml fractions with GST elution buffer. The fractions were analyzed by 15% SDS-PAGE (Laemmli) and visualized by staining with Coomassie Brilliant Blue R250 stain.

C. Affinity Column Preparation.

GST, and GST ORF168 were dialyzed overnight against ACB at pH 7.9 containing 1 M NaCl. Protein concentrations were determined by Bio-Rad Protein Assay and crosslinked to Affigel 10 resin (Bio-Rad) at protein/resin concentrations of 0, 0.1, 0.5, 1.0, and 2.0 mg/ml. The resin containing crosslinked protein was washed with ACB containing 1M NaCl and equilibrated with ACB containing 100 mM NaCl.

D. S. aureus Extract Preparation.

Staphylococcus aureus extract was prepared from a cell pellet using a bead beater lysis method and nuclease treatment. A Staphylococcus aureus cell pellet (2.9 g) was suspended in 8 ml of 20 mM Hepes pH 7.5, 500 mM NaCl, 10% glycerol, 10 mM MgSO₄, 10 mM CaCl₂, 1 mM DTT, 1 mM PMSF, 1 mM benzamidine, 0.5 mg RNAse A, and 750 units micrococcal nuclease. The cell suspension was added to 15 ml of zirconia/silica glass beads (0.1 mm diameter) at 4° C., and subjected to bead beater pulses (30 seconds on, 90 seconds off, using a super-cooled ice bath at −18° C.). The beads were separated from the lysate with a Biorad Econo column (2.5×40 cm) and the beads washed with one column volume of lysis buffer. The flow through and the wash were pooled and centrifuged at 20 000 rpm for 1 hrs in a Beckman JA25.50 rotor. The supernatant was removed and dialyzed overnight in a 10 000 Mr dialysis membrane against ACB (20 mM Hepes pH 7.5, 10% glycerol, 1 mM DTT, and 1 mM EDTA) containing 100 mM NaCl, 1 mM benzamidine, 10 mM MgSO₄, 10 mM CaCl₂, and 1 mM PMSF. The dialyzed protein extract was removed from the dialysis tubing and frozen in one ml aliquots at −70° C.

E. Affinity Chromatography

Affinity chromatography was performed using GST and GST ORF168 as ligands coupled to Affigel 10 at protein/resin concentrations of 0, 0.1, 0.5, 1.0, and 2.0 mg/ml. Staphylococcus aureus extracts were centrifuged at 4° C. in a micro-centrifuge for 15 minutes and diluted to 5 mg/ml with ACB containing 100 mM NaCl. 200 μl of extract was applied to 40 μl columns containing 0, 0.1, 0.5, 1.0, and 2.0 mg/ml ligand and ACB containing 100 mM NaCl (200 μl) was applied to an additional column containing 2.0 mg/ml ligand. The columns were washed twice with ACB containing 100 mM NaCl (2×100 μl), ACB containing 0.1% Triton X-100 and 100 mM NaCl (200 μl), and sequentially eluted with ACB containing 1 M NaCl (160 μl), and 1% SDS (160 μl). 65 μl of each eluate was resolved by 16 cm 14% SDS-PAGE (Laemmli) and the protein was visualized by silver stain.

F. Identification of S. Aureus DnaN as a Twort ORF 168 Interacting Protein

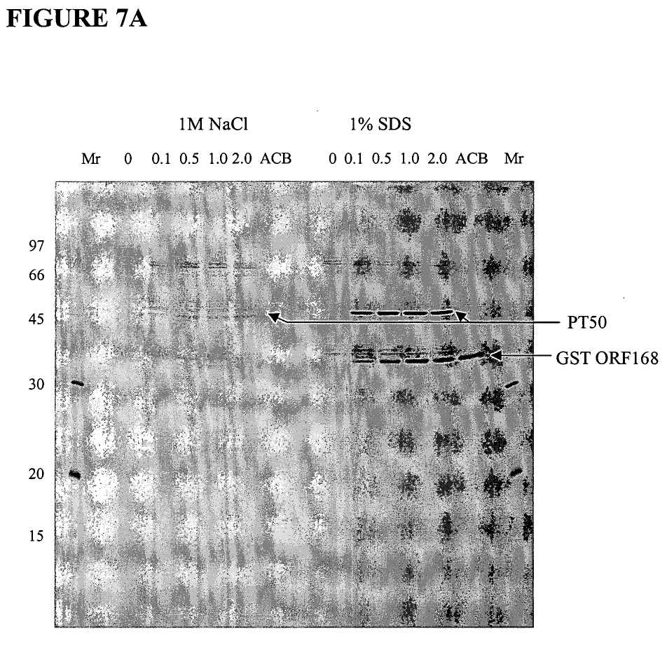

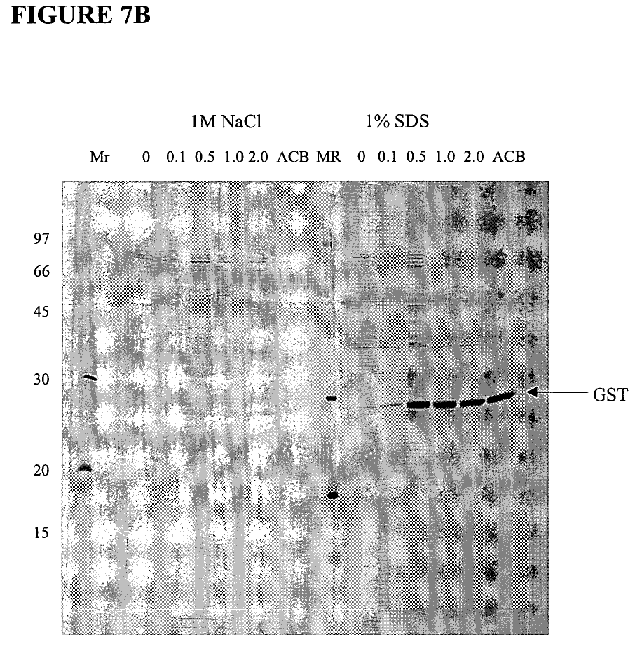

One candidate interacting protein of 50 kDa (PT50) was observed to interact with ORF 168. PT50 was observed in both the 1 M NaCl eluates, and the 1% SDS eluates of the GST ORF168 chromatography experiment (FIG. 7A). Approximately 10% of the PT50 was observed in the 1 M NaCl eluates while 90% was observed in the 1% SDS eluates. PT50 was not observed in the GST control affinity chromatography experiment (FIG. 7B).

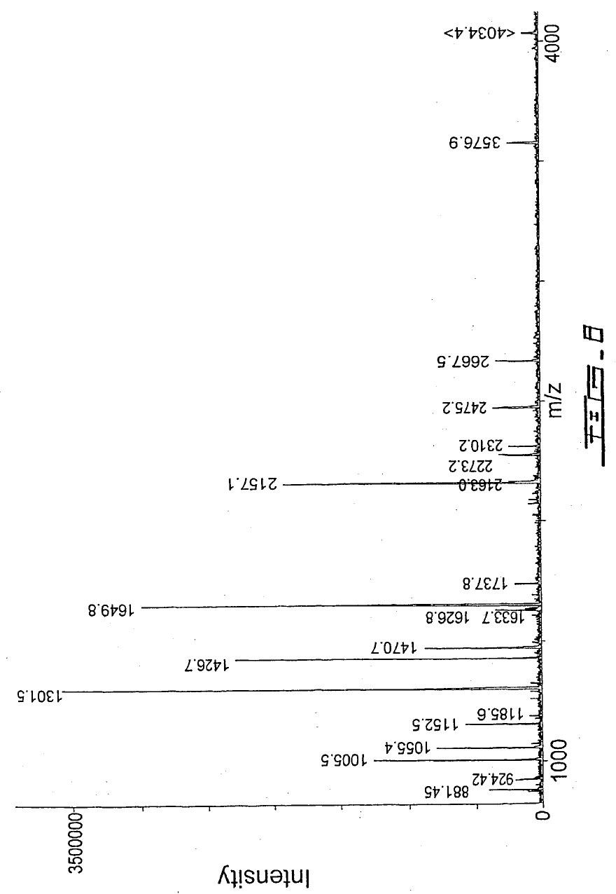

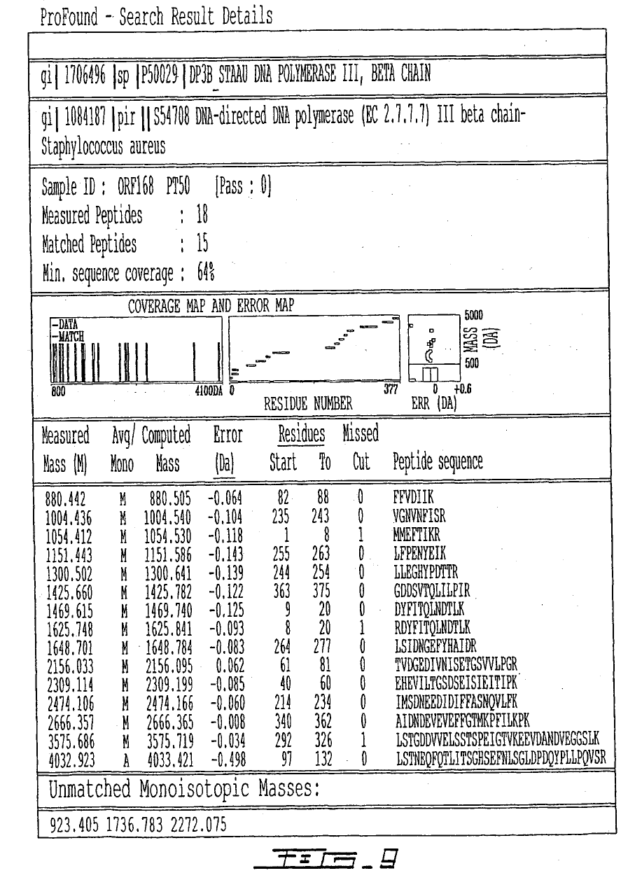

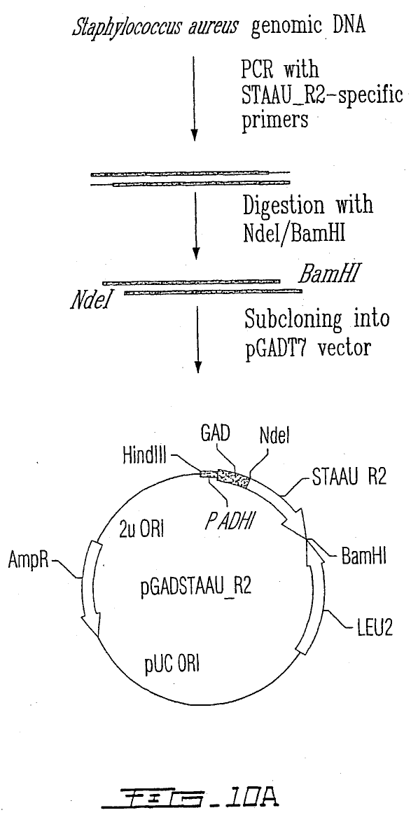

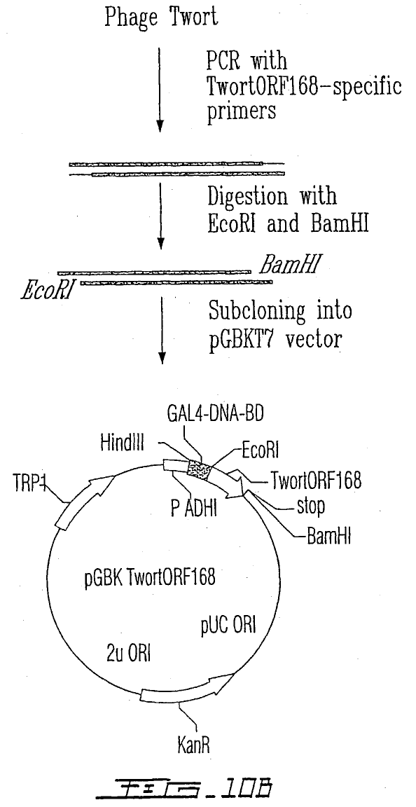

High quality mass spectra were obtained (FIG. 8). The PT50 proteins observed in affinity chromatography experiments were identical as determined by the masses of the tryptic peptides. The identity of that protein which binds specifically to GST ORF 168 is the DNA-directed DNA polymerase III beta subunit (Genbank accession #1084187) (FIG. 9).

### EXAMPLE 4

### Confirmation of the Interaction between STAAU_R2 and Twort ORF168 by Yeast Two-Hybrid Analysis

To validate the identification of S. aureus STAAU_R2 as an interacting partner of bacteriophage Twort ORF 168 and to identify a specific domain of Twort ORF 168 which participates in the interaction with S. aureus STAAU_R2, recombinant Twort ORF 168 protein was subjected to deletion analysis using the yeast two-hybrid system.

A. Generation of Twort ORF 168 and STAAU_R2 Recombinant Polypeptides for Yeast Two-Hybrid Analysis.

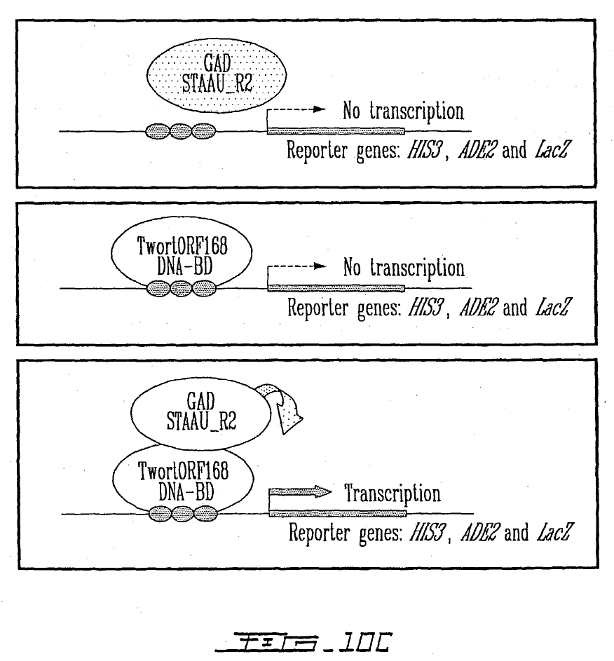

Bacteriophage Twort ORF 168 was fused either to the carboxyl terminus of the yeast Gal4 DNA binding domain (encoded in the pGBKT7 vector from Clontech Laboratories) or to the yeast Gal4 activation domain (encoded in the pGADT7 vector from Clontech Laboratories). As illustrated in FIG. 10B, the sense strand primer (SEQ ID NO: 11; 5-ccggaattcATGTTATTTTTTAAAGAAAAG-3′) is preceded by a EcoRI restriction site; the antisense oligonucleotide (SEQ ID NO: 12; 5′-cgcggatccTCATCGMCTATATCCTTAAT-3′) targets the stop codon and is preceded by a BamHI restriction site. The PCR product was purified using the Qiagen PCR purification kit and digested with EcoRI and BamHI. The digested PCR product was ligated to EcoRI- and BamHI-digested pGBKT7 vector, yielding pGBKSTAAU_R2. A similar strategy was used for the cloning of STAAU_R2 into the pGADT7 vector yielding pGADSTAAU_R2.

Fragment of Twort ORF 168 extending from amino acid residues 5 to 40 was amplified by PCR and cloned using the same strategy. The sense strand primer (SEQ ID NO: 13; 5-ccggaattcAAAGAAAAGTTTTATAATGAAT-3′) targets the initiation codon and is preceded by a EcoRI restriction site; the antisense oligonucleotide (SEQ ID NO: 14; 5′-cgcggatccTCAATCTTCTTCTTCTAATTTCTC-3′) targets the stop codon and is preceded by a BamHI restriction site.

The polynucleotide sequence of STAAU_R2 was obtained from S. aureus genomic DNA by PCR utilizing oligonucleotide primers that targeted the predicted translation initiation and termination codons of the STAAU_R2 gene (SEQ ID NO: 1).

As illustrated in FIG. 10A, the sense strand primer (SEQ ID NO: 15; 5-GGGAATTCCATATGATGATGGAATTCACTATTAAA-3′) targets the initiation codon and is preceded by a NdeI restriction site; the antisense oligonucleotide (SEQ ID NO: 16; 5′-CGCGGATCC TTAGTAAGTTCTGATTGG-3′) targets the stop codon and is preceded by a BamHI restriction site. The PCR product was purified using the Qiagen PCR purification kit and digested with NdeI and BamHI. The digested PCR product was ligated to NdeI- and BamHI-digested pGADT7 vector (Clontech Laboratories), yielding pGADSTAAU_R2. A similar strategy was used for the cloning of STAAU_R2 into the pGBKT7 vector (Clontech Laboratories), yielding pGBKSTAAU_R2.

B. Yeast Two-Hybrid Analysis

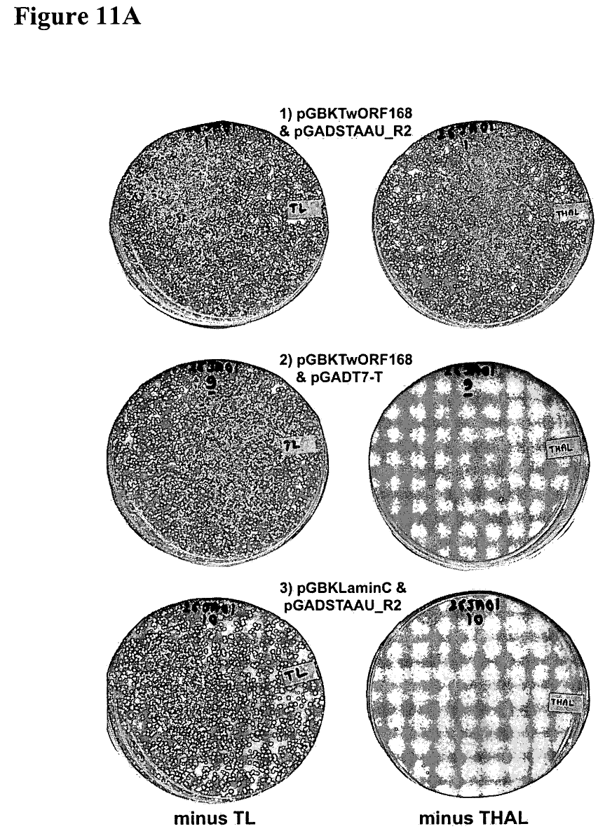

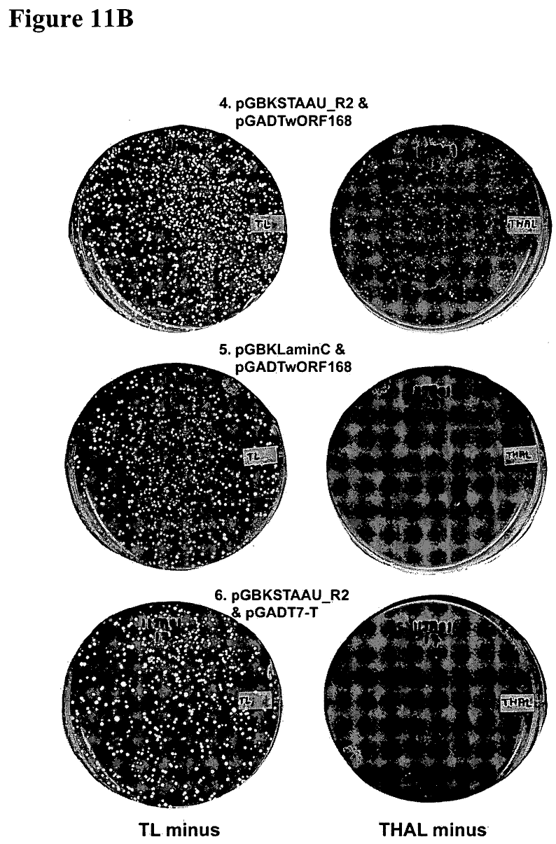

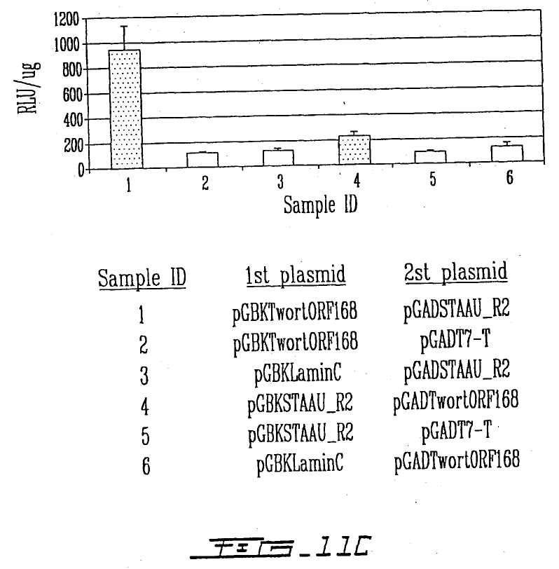

As shown in FIGS. 11A and B, the pGAD and pGBK plasmids bearing different combinations of constructs (as indicated above each pair of petri plates) were introduced into a yeast strain (AH109, Clontech Laboratories), previously engineered to contain chromosomally-integrated copies of E. coli lacZ and the selectable HIS3 and ADE2 genes. Co-transformants were plated in parallel on yeast synthetic medium (SD) supplemented with amino acid drop-out lacking tryptophan and leucine (TL minus) and on SD supplemented with amino acid drop-out lacking tryptophan, histidine, adenine and leucine (THAL minus). Co-transformants harbouring the Twort ORF 168 polypeptide only grew on selective SD THAL minus medium in the presence of STAAU_R2. Induction of the reporter HIS3 and ADE2 genes is dependent upon the interaction of STAAU_R2 with Twort ORF 168 since cotransformants with appropriate control plasmids (pGBKT7LaminC or pGADT7-T) are not viable on SD THAL minus medium.

The interaction of STAAU_R2 and Twort ORF 168 was also clearly demonstrated by the observed increase, over the background level, of the β-galactosidase activity in both Twort ORF 168-STAAU_R2 co-transformants (FIG. 11C samples 1 and 4 respectively). These results are consistent with the interpretation that the S. aureus STAAU_R2 identified herein is the host target of bacteriophage Twort ORF 168.

The interaction of STAAU_R2 and a Twort ORF 168-related fragment was also demonstrated (results not shown). A portion of 36 amino acid sequence of Twort ORF 168 extending from amino acids residues 5 to 40 (herein referred to as SEQ ID NO: 8) was found to interact with STAAU_R2 since the introduction of appropriate plasmids into host yeast cells resulted in their growth on THAL minus SD medium.

### EXAMPLE 5

### Identification of STAAU_R2 as Targeted by Bacteriophage G1 ORF 240

A. Generation of GST/G1 ORF 240 Recombinant Protein

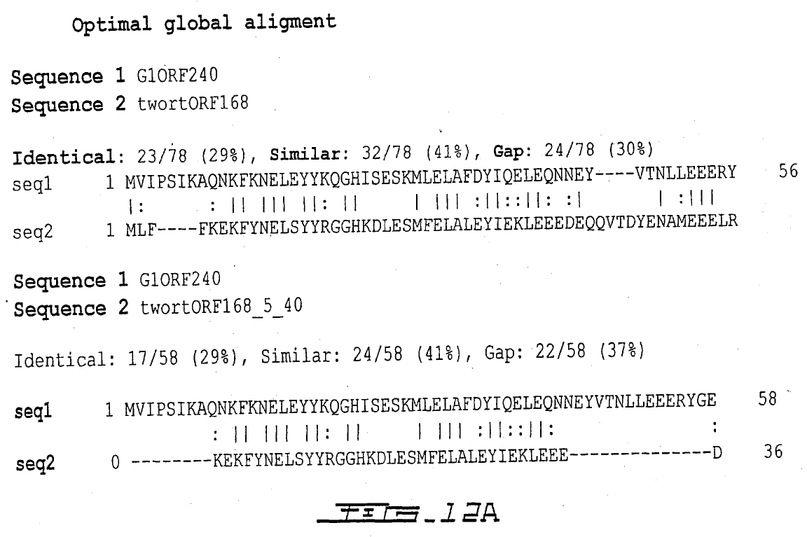

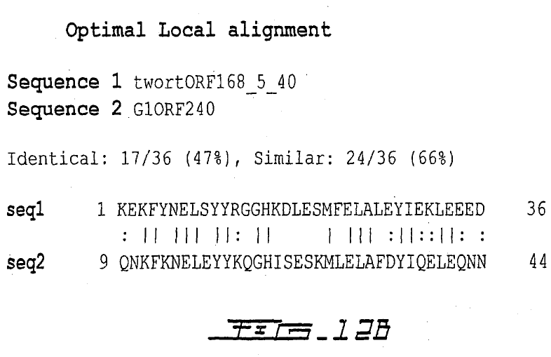

As shown in FIG. 12, the optimal global aligment of G1 ORF 240 and Twort ORF 168 reveals an identity of 29% between the two polypeptides. The optimal local aligment of G1 ORF 240 shows a 47% identity to the polypeptide corresponding to SEQ ID NO: 8.

G1 ORF 240 was cloned into pGEX 4T-1 and the polypeptide was purified as described above. Affinity chromatography was performed using GST and GST ORF240 as ligands coupled to Affigel 10 at protein/resin concentrations of 0, 0.1, 0.5, 1.0, and 2.0 mg/ml. Staphylococcus aureus extracts was applied to columns and ACB containing 100 mM NaCl was applied to an additional column containing 2.0 mg/ml ligand. The columns were washed and sequentially eluted with ACB containing 1 M NaCl, and 1% SDS. A fraction of each eluate was resolved by 14% SDS-PAGE and the protein was visualized by silver stain.

One candidate interacting protein was observed to specifically interact with ORF 240 and was shown to correspond to STAAU_R2 (results not shown).

### CONCLUSION

By virtue of the interaction between the inhibitory bacteriophage 44AHJD ORF 25, Twort ORF 168, G1 ORF 240_and the STAAU_R2, the STAAU_R2 gene and its gene product have thus been identified as novel bacterial targets for the screening and identification of anti-bacterial agents and more particularly for anti S. aureus agents. The present invention also provides novel diagnosis, prognosis and therapeutic methods based on STAAU_R2, and/or bacteriophage 44AHJD ORF 25 and/or Twort ORF 168, G1 ORF 240 and/or a compound identified in accordance with the present invention.

Although the present invention has been described hereinabove by way of preferred embodiments thereof, it can be modified without departing from the spirit and nature of the subject invention as defined in the appended claims.

What is claimed is:

1. A method for identifying a compound that is active on a STAAU_R2 polypeptide which comprises the amino acid sequence of SEQ ID NO: 2, a biologically active fragment thereof, or variant thereof, wherein said amino acid sequence of SEQ ID NO:2, biologically active fragment thereof, or variant thereof is capable of binding specifically to a bacteriophage polypeptide sequence, said method comprising: contacting a STAAU_R2 polypeptide in the presence or absence of a candidate compound, and detecting a biological activity of said STAAU_R2 polypeptide, wherein a decrease in the biological activity thereof in the presence of the candidate compound relative to the biological activity in the absence thereof identifies the candidate compound as a compound that is active on the STAAU_R2 polypeptide.

2. The method of claim 1, wherein said bacteriophage polypeptide sequence is selected from the group consisting of:
a) SEQ ID NO:4;
b) SEQ ID NO:6;
c) SEQ ID NO:8;
d) SEQ ID NO:10; and
e) a fragment or variant of any one of a) to d), wherein the fragment or variant thereof maintains its specific binding capability of interacting with SEQ ID NO:2, fragment or variant thereof.

3. The method of claim 2, wherein said detecting comprises the act of measuring the binding of said STAAU_R2 polypeptide to said bacteriophage polypeptide wherein said STAAU_R2 polypeptide or said bacteriophage polypeptide is directly or indirectly detectably labeled.

4. The method of any one of claims 1 to 3, wherein said detecting comprises measurement by FRET.

5. The method of any one of claims 1 to 3, wherein said detecting comprises measurement of fluorescence polarization changes.

6. The method of any one of claims 1 to 3, wherein said detecting comprises measurement by surface plasmon resonance.

7. The method of any one of claims 1 to 3, wherein said detecting comprises a scintillation proximity assay.

8. The method of any one of claims 1 to 3, wherein said detecting comprises a biosensor assay.

9. The method of any one of claims 1 to 3, wherein said detecting comprises measurement by phage display.

10. The method of any one of claims 1 to 3, wherein said candidate compound is selected from the group consisting of a small molecule, a peptidomimetic compound, and a fragment or derivative of a bacteriophage inhibitor protein.

11. The method of any one of claims 1 to 3, wherein said candidate compound is a peptide synthesized by an expression system and purified, or artificially synthesized.

12. A method for identifying a compound active on one of a STAAU_R2 polypeptide, or on a polypeptide derived from a bacteriophage ORF which specifically interacts with said STAAU_R2 polypeptide comprising: contacting a first and a second polypeptide in the presence or absence of a candidate compound, wherein said first polypeptide is a STAAU_R2 polypeptide which comprises the amino acid sequence of SEQ ID NO: 2, fragment, or variant thereof, and wherein said second polypeptide is a bacteriophage ORF selected from the group consisting of:
a) SEQ ID NO:4;
b) SEQ ID NO:6;
c) SEQ ID NO:8;
d) SEQ ID NO:10; and
e) a fragment or variant of any one of a) to d), wherein the fragment or variant thereof maintains its biological activity; and detecting a biological activity of the first and/or second polypeptide, wherein a decrease in the biological activity thereof in the presence of the candidate compound relative to in the absence thereof identifies the candidate compound as a compound that is active on one of said STAAU_R2 polypeptide or a polypeptide derived from a bacteriophage.

13. The method of claim 12, which identifies a compound active on STAAU_R2.

14. The method of claim 12 or 13, wherein said detecting comprises the step of measuring the binding of a candidate compound to said polypeptide, wherein the compound is directly or indirectly detectably labeled.

15. The method of claim 12 or 13, wherein said detecting comprises measurement by FRET.

16. The method of claim 12 or 13, wherein said detecting comprises measurement of fluorescence polarization changes.

17. The method of claim 12 or 13, wherein said detecting comprises measurement by surface plasmon resonance.

18. The method of claim 12 or 13, wherein said detecting comprises a scintillation proximity assay.

19. The method of claim 12 or 13, wherein said detecting comprises a biosensor assay.

20. The method of claim 12 or 13, wherein said detecting comprises measurement by phage display.

21. The method of claim 12 or 13, wherein said active compound is selected from the group consisting of a small molecule, a peptidomimetic compound, and a fragment or derivative of a bacteriophage inhibitor protein.

22. The method of claim 12 or 13, wherein said active compound is a peptide synthesized by an expression system and purified, or artificially synthesized.

23. An agonist or an antagonist of the activity of a STAAU_R2 polypeptide or fragment thereof, or a nucleic acid encoding said polypeptide or fragment thereof.

24. A method of identifying a compound that is active on a STAAU_R2 polypeptide, said method comprising:
contacting a candidate compound with cells expressing a polypeptide comprising the amino acid sequence of SEQ ID NO: 2, fragment or variant thereof, wherein said amino acid sequence of SEQ ID NO:2, fragment thereof, or variant thereof is capable of binding specifically to a bacteriophage polypeptide sequence, and
detecting a STAAU_R2 activity in said cells, wherein a decrease in said activity in said cells in the presence of said candidate compound is indicative of an inhibition of STAAU_R2 activity by said compound.

25. A method of making an antibacterial compound, comprising the steps of:
determining whether a candidate compound is active on a polypeptide comprising the amino acid sequence of SEQ ID NO: 2, fragment thereof or variant thereof, or a nucleic acid encoding said polypeptide, wherein said amino acid sequence of SEQ ID NO:2, biologically active fragment thereof, or variant thereof is capable of binding specifically to a bacteriophage polypeptide sequence;
synthesizing or purifying said candidate compound in an amount sufficient to provide a therapeutic effect when administered to an organism infected by a bacterium naturally producing said polypeptide, or nucleic acid encoding same.

26. The method of claim 25, wherein the antibacterial compound is selected from the group consisting of a small molecule, a peptidomimetic compound, and a fragment or derivative of a bacteriophage inhibitor protein.

27. The method of claim 25, wherein the antibacterial compound is a peptide synthesized by an expression system and purified, or artificially synthesized.

28. A method for inhibiting a bacterium, comprising contacting the bacterium with a compound active on a S. aureus polypeptide comprising the amino acid sequence of SEQ ID NO: 2, or fragment or variant thereof, or a nucleic acid encoding same.

29. The method of claim 28, wherein said compound is selected from the group consisting of:
a) SEQ ID NO:4;
b) SEQ ID NO:6;
c) SEQ ID NO:8;
d) SEQ ID NO:10; and
e) a fragment or variant of any one of a) to d), wherein the fragment or variant thereof maintains its specific binding capability of interacting with SEQ ID NO:2, fragment or variant thereof.

30. The method of claim 29, wherein said contacting is performed in vitro.

31. The method of claim 29, wherein said contacting is performed in vivo in an animal.

32. The method of claim 29, wherein said contacting is performed in combination with existing antimicrobial agents.

33. The method of claim 29, wherein the antibacterial compound is selected from the group consisting of a small molecule, a peptidomimetic compound, and a fragment or derivative of a bacteriophage inhibitor protein.

34. The method of claim 29, wherein the antibacterial compound is a peptide synthesized by an expression system and purified, or artificially synthesized.

35. A method for treating or preventing a bacterial infection in an animal suffering from an infection, comprising administering to the animal a therapeutically effective or prophylactic effective amount of a compound active on a S. aureus polypeptide comprising the amino acid sequence of SEQ ID NO: 2, fragment or variant thereof, or a nucleic acid encoding said polypeptide.

36. The method of claim 36, wherein said compound is selected from the group consisting of:
a) SEQ ID NO:4;
b) SEQ ID NO:6;
c) SEQ ID NO:8;
d) SEQ ID NO:10; and
e) a fragment or variant of any one of a) to d), wherein the fragment or variant thereof maintains its specific binding capability of interacting with SEQ ID NO:2, fragment or variant thereof.

37. A method of prophylactic treatment to prevent bacterial infection comprising contacting an indwelling device with a compound active on a S. aureus polypeptide comprising the amino acid sequence of SEQ ID NO: 2, fragment thereof or variant thereof, before its implantation into a mammal, such contacting being sufficient to prevent S. aureus infection at the site of implantation.

38. A method of prophylactic treatment to prevent infection of an animal by a bacterium comprising administering to the animal a compound that is active on a S. aureus polypeptide comprising the amino acid sequence of SEQ ID NO: 2, or fragment thereof or variant thereof, or a gene encoding said polypeptide in an amount sufficient to reduce adhesion of the bacterium to a tissue surface of a tissue of the mammal.

39. A composition comprising a STAAU_R2 polypeptide, fragment or variant thereof, and at least one bacteriophage polypeptide selected from a bacteriophage 44AHJD ORF 25, Twort ORF 168-encoded polypeptide, and G1 ORF 240, or a fragment from said bacteriophage polypeptide.

40. A composition comprising two specifically interacting domains, wherein said first domain is derived from a STAAU_R2 polypeptide and said second domain is derived from a polypeptide encoded by a bacteriophage ORF which specifically interacts with said STAAU_R2 polypeptide.

41. A process for producing a pharmaceutical composition comprising: a) carrying out a screening assay of the present invention aimed at identifying a compound that is active on a STAAU_R2 polypeptide comprising the amino acid sequence of SEQ ID NO:2, a biologically active fragment thereof, or variant thereof, wherein said STAAU_R2 polypeptide is capable of binding specifically to a second polypeptide derived from a bacteriophage ORF, and wherein the screening assay enables the identification of a candidate compound as a compound that is active on a STAAU_R2 polypeptide when a biologically activity of said STAAU_R2 polypeptide is measurably different in the presence of said candidate compound as compared to in the absence thereof; and b) mixing the compound identified in a) in a pharmaceutically effective amount with a suitable pharmaceutical carrier, thereby producing a pharmaceutical composition.

42. Use of one of: a) a STAAU_R2 polypeptide comprising the amino acid sequence of SEQ ID NO:2, a biologically active fragment thereof or variant thereof, wherein said STAAU_R2 polypeptide is capable of binding specifically to a polypeptide derived from a bacteriophage ORF, b) a composition comprising a pair of specifically interacting domains comprised of a polypeptide of STAAU_R2, biologically active fragment thereof or variant thereof and a polypeptide encoded by a bacteriophage ORF which specifically interacts with STAAU_R2; or c) an assay mixture comprising a first polypeptide which comprises the amino acid sequence of SEQ ID NO:2, biologically active fragment thereof or variant thereof and a second polypeptide encoded by a bacteriophage ORF which specifically interact with each other; for the identification of a compound that is active on a polypeptide comprising the amino acid sequence of SEQ ID NO:2, biologically active fragment thereof or variant thereof.

43. Use according to claim 42, wherein said compound active on said polypeptide is used for the manufacture of an antibacterial agent or for the manufacture of a medicament for treating or preventing a bacterial infection.


SEQUENCE LISTING (16 sequences)

SEQ ID NO: 1 - DNA, 1134 bp, Staphylococcus aureus
  CDS (1)..(1134)
atgatggaattcactattaaaagagattattttattacacaattaaatgacacattaaaagctatttcaccaagaacaacattacctatattaactggtatcaaaatcgatgcgaaagaacatgaagttatattaactggttcagactctgaaatttcaatagaaatcactattcctaaaactgtagatggcgaagatattgtcaatatttcagaaacaggctcagtagtacttcctggacgattctttgttgatattataaaaaaattacctggtaaagatgttaaattatctacaaatgaacaattccagacattaattacatcaggtcattctgaatttaatttaagtggcttagatccagatcaatatcctttattacctcaagtttctagagatgacgcaattcaattgtcggtaaaagtgcttaaaaacgtgattgcacaaacaaattttgcagtgtccacctcagaaacacgcccagtactaactggtgtgaactggcttatacaagaaaatgaattaatatgcacagcgactgactcacaccgcttggctgtaagaaagttgcagttagaagatgtttctgaaaacaaaaatgtcatcattccaggtaaggctttagctgaattaaataaaattatgtctgacaatgaagaagacattgatatcttctttgcttcaaaccaagttttatttaaagttggaaatgtgaactttatttctcgattattagaaggacattatcctgatacaacacgtttattccctgaaaactatgaaattaaattaagtatagacaatggggagttttatcatgcgattgatcgtgcctctttattagcgcgtgaaggtggtaataacgttattaaattaagtacaggtgatgacgttgttgaattgtcttctacatcaccagaaattggtactgtaaaagaagaagttgatgcaaacgatgttgaaggtggtagcctgaaaatttcattcaactctaaatatatgatggatgctttaaaagcaatcgataatgatgaggttgaagttgaattcttcggtacaatgaaaccatttattctaaaaccaaaaggtgacgactcggtaacgcaattaattttaccaatcagaacttactaa

SEQ ID NO: 2 - protein, 377 aa, Staphylococcus aureus
MMEFTIKRDYFITQLNDTLKAISPRTTLPILTGIKIDAKEHEVILTGSDSEISIEITIPKTVDGEDIVNISETGSVVLPGRFFVDIIKKLPGKDVKLSTNEQFQTLITSGHSEFNLSGLDPDQYPLLPQVSRDDAIQLSVKVLKNVIAQTNFAVSTSETRPVLTGVNWLIQENELICTATDSHRLAVRKLQLEDVSENKNVIIPGKALAELNKIMSDNEEDIDIFFASNQVLFKVGNVNFISRLLEGHYPDTTRLFPENYEIKLSIDNGEFYHAIDRASLLAREGGNNVIKLSTGDDVVELSSTSPEIGTVKEEVDANDVEGGSLKISFNSKYMMDALKAIDNDEVEVEFFGTMKPFILKPKGDDSVTQLILPIRTY

SEQ ID NO: 3 - DNA, 177 bp, Bacteriophage 44AHJD
  CDS (1)..(177)
atggaacgtaaatacaaaacggtattattatattgcgatgagattaaaggacattttccacatcaaatctcaatgtttgaagatttatatgacgctaaagttgtatattcatattatgaatataacctgttcactaaaaaatacgcgtatatcatagaatacattaaggagatataa

SEQ ID NO: 4 - protein, 58 aa, Bacteriophage 44AHJD
MERKYKTVLLYCDEIKGHFPHQISMFEDLYDAKVVYSYYEYNLFTKKYAYIIEYIKEI

SEQ ID NO: 5 - DNA, 225 bp, Bacteriophage Twort
  CDS (1)..(225)
atgttattttttaaagaaaagttttataatgaattaagttattatagaggtggacacaaggatttagaaagtatgtttgagttagcgttagagtatattgagaaattagaagaagaagatgaacagcaggtaactgattatgagaacgctatggaagaagaattaagggatgctgttgatgtaattgagagtcagttagaaattattaaggatatagttcgatga

SEQ ID NO: 6 - protein, 74 aa, Bacteriophage Twort
MLFFKEKFYNELSYYRGGHKDLESMFELALEYIEKLEEEDEQQVTDYENAMEEELRDAVDVIESQLEIIKDIVR

SEQ ID NO: 7 - DNA, 108 bp, Bacteriophage Twort
  CDS (1)..(108)
aaagaaaagttttataatgaattaagttattatagaggtggacacaaggatttagaaagtatgtttgagttagcgttagagtatattgagaaattagaagaagaagat

SEQ ID NO: 8 - protein, 36 aa, Bacteriophage Twort
KEKFYNELSYYRGGHKDLESMFELALEYIEKLEEED

SEQ ID NO: 9 - DNA, 177 bp, Bacteriophage G1
  CDS (1)..(177)
atggttatacctagtattaaagcacaaaacaaattcaagaatgagttagagtattataaacaaggtcacattagtgaaagtaaaatgttagaattagcttttgattacatccaagaattagaacaaaataacgaatacgttactaacttgctagaagaggagagatatggtgagtaa

SEQ ID NO: 10 - protein, 58 aa, Bacteriophage G1
MVIPSIKAQNKFKNELEYYKQGHISESKMLELAFDYIQELEQNNEYVTNLLEEERYGE

SEQ ID NO: 11 - DNA, 30 bp, artificial sequence
  Sequence is completely synthesized
ccggaattcatgttattttttaaagaaaag

SEQ ID NO: 12 - DNA, 30 bp, artificial sequence
  Sequence is completely synthesized
cgcggatcctcatcgaactatatccttaat

SEQ ID NO: 13 - DNA, 31 bp, artificial sequence
  Sequence is completely synthesized
ccggaattcaaagaaaagttttataatgaat

SEQ ID NO: 14 - DNA, 33 bp, artificial sequence
  Sequence is completely synthesized
cgcggatcctcaatcttcttcttctaatttctc

SEQ ID NO: 15 - DNA, 35 bp, artificial sequence
  Sequence is completely synthesized
gggaattccatatgatgatggaattcactattaaa

SEQ ID NO: 16 - DNA, 27 bp, artificial sequence
  Sequence is completely synthesized
cgcggatccttagtaagttctgattgg